(19) United States
(12) Patent Application Publication
(10) Pub. No.: US 2024/0264161 A1
(43) Pub. Date: Aug. 8, 2024

(54) BIOMARKERS AND USES THEREOF

(71) Applicant: MetaCurUm Biotech AB, Umeå (SE)

(72) Inventors: Maréne LANDSTRÖM, Umeå (SE); Jie Song, Umeå (SE)

(21) Appl. No.: 18/566,433

(22) PCT Filed: May 20, 2022
(86) PCT No.: PCT/EP2022/063820

§ 371 Date: Dec. 1, 2023

(30) Foreign Application Priority Data
Jun. 3, 2021 (SE) 2150710-8

(51) Int. Cl. G01N 33/574

(52) U.S. Cl. CPC G01N 33/57496; G01N 2333/912

(57) ABSTRACT

The present invention provides methods for classifying, diagnosing, and monitoring a subject having a cancer through the measurement of novel biomarkers which co-localize. Also provided are kits and arrays for diagnosing cancer, specifically aggressive cancer; differential diagnosis; and monitoring the progression of cancer.

### TECHNICAL FIELD

The present invention provides methods for classifying, diagnosing, and monitoring a subject having a cancer through the measurement of novel biomarkers which co-localize. Also provided are kits and arrays for diagnosing cancer, specifically aggressive cancer; differential diagnosis; and monitoring the progression of cancer.

### BACKGROUND

Transforming growth factor β (TGFβ) is overexpressed in several advanced cancers and promotes tumor progression. How cancer cells evade TGFβ-induced growth inhibition and escape normal homeostasis is unclear. In the canonical TGFβ-Smad signaling pathway, cellular responses depend on the kinase activity of TGFβ receptor I (TβRI), leading to the formation of Smad2, Smad3, and Smad4 complexes that regulate the transcription of certain genes, including SERPINE1, Snail1, and metalloproteinase protein 2. TβRI is cleaved in its extracellular domain by TNF-α converting enzyme (TACE/ADAM17), resulting in a loss of growth inhibitory effects mediated by TGFβ mediated by Smad-proteins (Liu C et al. Mol Cell 2009; 35(1): 26-36).

In contrast, in non-canonical TGFβ-induced signaling pathways, cellular responses are often regulated by the E3-ligase tumor necrosis factor receptor-associated factor 6 (TRAF6). This protein associates with TβRI and is activated upon ligand binding to receptors, promoting activation of the MAP kinase kinase kinase TGFβ-activated kinase 1 (TAK1). TRAF6 promotes activation of the phosphatidylinositol-3′-kinase (PI3K)-AKT pathway in response to insulin stimulation through K63-linked polyubiquitination of the endosomal protein Adaptor Protein, Phosphotyrosine Interacting With PH Domain And Leucine Zipper 1 (APPL1) on K160^{11, 13-15}, and in response to TGFβ stimulation, by K63-linked polyubiquitination of the regulatory subunit p85α in the PI3K complex (Hamidi A, et al. Sci Signal 2017; 10(486)). TRAF6 also activates proteolytic enzymes, such as ADAM17/TACE and presenilin 1 in the γ-secretase complex, to cleave off the intracellular domain (ICD) of TβRI, allowing soluble TβRI-ICD to enter the nucleus, after ubiquitination of K178 by TRAF6, to promote transcription of pro-invasive genes and TGFBR1.

The inventors have recently shown that the endosomal adaptor proteins APPL1 and APPL2 associate with TβRI-ICD and enhance nuclear accumulation of TβRI-ICD in response to TGFβ stimulation of cells, promoting invasiveness of prostate cancer cells in vitro and showing a strong correlation with aggressiveness of human prostate cancers (Song J, Mu Y, Li C, Bergh A, Miaczynska M, Heldin C-H, et al. APPL proteins promote TGFβ-induced nuclear transport of the TGFβ type I receptor intracellular domain. Oncotarget. 2016; 7:279-92).

WO 2012/125623 discloses the use of cleavage inhibitors of TβRI and uses thereof in cancer therapy, and a diagnostic method, wherein nuclear localization of the TβRI-ICD indicates presence of cancer cells in the sample, and the likelihood of cancer invasiveness/metastasis in the subject.

The TGFβ signaling pathway has dual and pivotal roles in tumor progression. In normal cells and at early stages of tumorigenesis, it acts as a tumor suppressor by inhibiting proliferation and inducing differentiation and apoptosis. TGFβ inhibits proliferation of several cell types, including epithelial and endothelial cells, keratinocytes, and leukocytes. In most normal cell types, TGFβ stimulation arrests cell cycle progression in G1 by downregulating expression of MYC and upregulating the expression of cyclin-dependent kinase inhibitors, including p15^{INK4B} and p21 (Sintich S M, Lamm M L, Sensibar J a, Lee C. Transforming growth factor-β1-induced proliferation of the prostate cancer cell line, TSU-Pr1: the role of platelet-derived growth factor. Endocrinology. 1999; 140:3411-5). However, in advanced cancers, when cancer cells evade the suppressive responses of TGFβ, the cytokine becomes a tumor promoter (i.e. TGFβ promotes tumorigenesis) by inducing epithelial-mesenchymal transition, facilitating tumor invasion and metastasis, and suppressing the immune system (Batlle E, Massague J. Transforming Growth Factor-β Signaling in Immunity and Cancer. Immunity 2019; 50: 924-940.).

Despite these findings, little is known about the role of TGFβ in mitosis. TGFβ can promote proliferation of certain mesenchymal and cancer cells, but its role in the mechanism of growth stimulation is poorly understood. As a stimulator of proliferation, TGFβ induces expression of fibroblast growth factor 2 in human renal fibroblasts, and platelet-derived growth factor in glioma and osteosarcoma cells. In normal prostatic epithelial cells, TGFβ acts as a growth suppressor by inhibiting proliferation and inducing apoptosis, whereas in prostate cancer cells, which have lost sensitivity to TGFβ-induced growth arrest, TGFβ may promote tumor cell growth. For example, TGFβ stimulates cell proliferation in the prostate cancer cell line TSU-Pr1 (Sintich S M, Lamm M L, Sensibar J a, Lee C. Transforming growth factor-β1-induced proliferation of the prostate cancer cell line, TSU-Pr1: the role of platelet-derived growth factor. Endocrinology. 1999; 140:3411-5), and causes only transient proliferation inhibition in the DU145 and PC-3 cell lines, while having no effect on proliferation of LNCaP prostate carcinoma cells (Wilding G, Zugmeier G, Knabbe C, Flanders K, Gelmann E. Differential effects of transforming growth factor β on human prostate cancer cells in vitro. Mol Cell Endocrinol. 1989; 62:79-87).

Aurora kinases are serine/threonine kinases that are essential for cell proliferation. They are phosphotransferase enzymes that help the dividing cell dispense its genetic materials to its daughter cells. More specifically, Aurora kinases play a crucial role in cellular division by controlling chromatid segregation. Aurora kinases, such as Aurora kinase A (AURKA) and Aurora kinase B (AURKB), are overexpressed in many tumors, including breast, lung, pancreatic, ovarian, and prostate tumors. Aurora kinase B (AURKB) is a component of the chromosomal passenger complex (CPC), which contains three regulatory components, i.e. the inner centromere protein (INCENP), survivin, and borealin. AURKB binds to the conserved C-terminal IN-box region of INCENP (Adams R R, et al. Curr Biol 2000; 10(17): 1075-8), where a Thr-Ser-Ser motif is located, which is phosphorylated by AURKB (Bishop J D, Schumacher J M. J Biol Chem 2002; 277(31): 27577-80), contributing to AURKB activation and stabilization of the complex. The AURKB: INCENP complex has also been suggested to favor autophosphorylation of AURKB in trans, as AURKB was found to form dimers in a study of its crystal structure (Elkins J M, et al. J Med Chem 2012; 55(17): 7841-8).

In interphase, CPC localizes in the heterochromatin, and after a cell enters mitosis, AURKB phosphorylation of histone H3 at Ser10 (H3S10) facilitates removal of CPC from the chromosome arms to the inner centromere. At anaphase onset, CPC releases from the chromosomes and re-localizes to the spindle midzone, where a phosphorylation gradient of AURKB is formed. During cytokinesis, CPC targets to the cleavage furrow and midbody. AURKB regulates abscission timing by controlling the localization and function of vacuolar protein sorting-associated protein 4 (VPS4) (5). Briefly, chromatin-modifying protein/charged multivesicular body protein (Chmp) 4c interacts with borealin and is phosphorylated by AURKB at several residues in a motif in the C-terminus which is missing in the Chmp4a and Chmp4b paralogs. In the midbody, Abscission/NoCut checkpoint regulator (ANCHR) interacts with Chmp4c and VPS4 to form a ternary complex. The kinase activity of AURKB is required to sustain this complex because treatment with an inhibitor of the AURKB kinase leads to the dissociation of VPS4 from Chmp4c (5). VPS4 is involved in the endosomal sorting complexes required for transport-III-mediated constriction and final scission. However, the regulation of the activity of VPS4 in abscission is still unknown. Because of their association with several different cancer types, inhibitors of Aurora kinases are being tested in clinical trials (Keen N, Taylor S. Aurora-kinase inhibitors as anticancer agents. Nat Rev Cancer. 2004; 4:927-36).

US 2016/0153052 relates to diagnostic assays useful in classification of patients for selection of cancer therapy with one or more Aurora kinase B inhibitors, either as monotherapy or as part of combination therapy, and monitoring patient response to such therapy, and CN110261612A relates to use of Aurora B and Survivin in preparing a colorectal cancer diagnostic kit.

Prostate cancer is the most common cancer in men worldwide, particularly in the Western countries, associated with around 375,000 deaths each year (Esfahani M, Ataei N, Panjehpour M. Biomarkers for Evaluation of Prostate Cancer Prognosis. 2015; 16:2601-11 and Sung H et al. CA Cancer J Clin 2021; 71(3):209-49). Transforming growth factor β (TGFβ) is a potent determinant of cell fate because of its contextual regulation of cell homeostasis and differentiation during embryogenesis and in several types of malignancies.

There are today no biomarkers available in tissues or body liquids, such as blood or urine for screening and detection of aggressive cancer. In prostate cancer, PSA (prostate specific antigen) is commonly used as a marker, but it is not reliable nor specific for prostate cancer. Prostate and renal (RCC) biopsies are assessed visually by pathologists and assigned a Gleason Score grade (prostate) or a Fuhrman grade in RCC. Both scores are subjective and dependent on the pathologists' experience. Moreover, there are currently no available tissue-based markers that can distinguish between a prostate cancer classified as Gleason score>7 and Gleason score<7. This is important as Gleason score (GS)>7 has worse prognosis than below 7 (Zhu et al., Front. Oncol., 16 Jul. 2019). Biomarkers are needed for patient selection/classification (to include only subjects able to respond to a specific treatment), verification of therapy mode of action and effectiveness, patient monitoring and assessing dose titration and product efficacy. This will accelerate the drug development process and reduce the number of patients needed in clinical trials, saving costs.

In view of the above, there is a need for novel biomarkers for diagnosing cancer in which the non-canonical TGFβ signaling pathways is involved, and thus classifying patients that would benefit for an anti-cancer treatment with an agent preventing this mechanism. There is also a need of biomarkers for predicting aggressive cancer at an early stage of the disease.

### SUMMARY

By knocking down expression of APPL1 and APPL2, the inventors surprisingly identified AURKB as a target gene for the APPL1/APPL2 regulated pathway in castration-resistant prostate cancer cells (CRPC). The inventors surprisingly found that TRAF6 was auto-ubiquitinated during mitotic progression and contributed to AURKB activity through K63-linked polyubiquitination of AURKB on K85 and K87. Moreover, the inventors surprisingly found that AURKB formed a complex with APPL1 and the intracellular domains of TβRI (TβRI-ICD) during mitosis and cytokinesis in CRPC cells, and in neuroblastoma cells a colocalization of AURKB and TβRI was observed by confocal imaging as well. The inventors surprisingly found APPL1 and TβRI were required for proliferation of CRPC cells. Moreover, high expression of AURKB and TβRI-ICD complexes visualized by in situ PLA technique was present in clinical prostate cancer material and correlated to poor prognosis. The inventors surprisingly found that the expression of AURKA and AURKB was higher in CRPC of neuroendocrine type than in CRPC adenocarcinoma, consistent with the poor prognosis for patients with CRPC of neuroendocrine type.

The present invention provides biomarkers for classifying, diagnosing, and monitoring a treatment of cancer in a subject. The biomarkers are also useful for identifying and predicting aggressive cancer forms.

Transforming growth factor β (TGFβ) is frequently overexpressed in several cancers, causing tumor progression. In-depth characterization of the functional significance of TβRI in mitosis demonstrates a newly identified, important role during cytokinesis.

A first object of the present invention provides a method for diagnosing cancer in a subject, the method comprising the steps of:
a) providing a biological test sample from the subject; and
b) determining the presence or absence of a first biomarker, a second biomarker, and a third biomarker, wherein said biomarkers are: Aurora kinase B (AURKB), Adaptor Protein, Phosphotyrosine Interacting With PH Domain And Leucine Zipper 1 (APPL1), and TGFβ receptor type 1 (TβR1), in the test sample;
   wherein the co-localization of all three biomarkers in the biological test sample is indicative of cancer in the subject.

Thus, it will be appreciated that step (b) may involve determining the co-localization of the first, second, and third biomarkers within the test sample. Examples of techniques that can be used to determine whether two proteins are co-localized include those described herein and include immunohistochemistry, in situ hybridization, immunoprecipitation, immunofluorescence, confocal microscopy, many of which are exemplified in the Examples.

For the avoidance of doubt, the co-localization of biomarkers does not require the biomarkers to be in a complex with each other, but merely that the biomarkers are spatially close to each other. For example, two proteins may be co-localized if they are observed as being spatially close to each other (for example, by immunofluorescence and digital imaging using z-stack), and a direct interaction between the biomarkers is not necessary. However, biomarkers may be co-localized because they do directly interact, and therefore both situations are encompassed by the term “co-localization”.

In an embodiment of all of the methods of the invention, the co-localization of the biomarkers to a cellular structure is indicative of cancer in the subject. Thus, it will be appreciated that step (b) may involve determining the co-localization of the first, second, and third biomarkers in a cellular structure within the test sample.

By “cellular structure” we include the meaning of any defined compartment or sub-compartment of a cell such as an organelle, including a sub-part of an organelle. Cellular structures include the nucleus, ribosomes, endoplasmic reticulum (ER), Golgi apparatus, cytoplasm and mitochondria. For example, the organelle may be the nucleus and the sub-part of the nucleus may be the midbody. Examples of techniques that can be used to determine whether two proteins are co-localized to a cellular structure are known in the art. For example, using immunofluorescence or immunohistochemistry, a marker for the nucleus may be used in addition to markers for the particular biomarkers, enabling the skilled person to assess whether these separate markers are all observed in the nucleus and thus whether the biomarkers are co-localized to the nucleus. Similarly, a population of cells may be fractionated and an immunoprecipitation may be carried out to determine whether the biomarkers are in a complex within, for example, the nuclear fraction.

In an embodiment of all of the methods of the invention, the cellular structure is the nucleus. In a further embodiment of all the methods of the invention, the cellular structure is a cytokinesis structure.

In an embodiment, the present invention provides a method for diagnosing cancer in a subject, the method comprising the steps of:
a) providing a biological test sample from the subject; and
b) determining the presence or absence of a first biomarker, a second biomarker, and a third biomarker, wherein said biomarkers are: Aurora kinase B (AURKB), Adaptor Protein, Phosphotyrosine Interacting With PH Domain And Leucine Zipper 1 (APPL1), and TGFβ receptor type 1 (TβR1), in the test sample;
   wherein the presence of all three biomarkers co-localized to a cytokinesis structure in the biological test sample is indicative of cancer in the subject.

Thus, it will be appreciated that step (b) may involve determining the presence or absence of the first, second, and third biomarkers in a cytokinesis structure within the test sample.

Thus, co-localization of three biomarkers to a cytokinesis structure includes the meaning of each of the three biomarkers being identifiable in one or more cytokinesis structures. In a particularly preferred embodiment, the cytokinesis structure is the midbody and so colocalization of the three biomarkers in a cytokinesis structure is colocalization of each of the three biomarkers to the midbody. For the avoidance of doubt, by co-localizing of biomarkers to a cytokinesis structure, it is not a requirement for the biomarkers to be in a complex with each other, but merely that the biomarkers are co-localized to a cytokinesis structure. For example, two proteins may be colocalized if they are observed as being close to each other by immunofluorescence and digital imaging using z-stack.

In an embodiment, the method further comprises determining the presence or absence of a fourth biomarker in the biological test sample, wherein said biomarker is TNF receptor associated factor 6 (TRAF6), and wherein the co-localization of all four biomarkers in the biological test sample is indicative of cancer in the subject.

In an embodiment of the methods of the invention, the co-localization of the biomarkers to a cellular structure is indicative of cancer in the subject. Thus, it will be appreciated that step (b) may involve determining the co-localization of the first, second, and third biomarkers in a cellular structure within the test sample.

In an embodiment, the method further comprises determining the presence or absence of a fourth biomarker in the biological test sample, wherein said biomarker is TNF receptor associated factor 6 (TRAF6), wherein the presence of all four biomarkers co-localized to a cytokinesis structure in the biological test sample is indicative of cancer in the subject.

Thus, co-localization of four biomarkers to a cytokinesis structure includes the meaning of each of the four biomarkers being identifiable in one or more cytokinesis structures. In a particularly preferred embodiment, the cytokinesis structure is the midbody and so colocalization of the four biomarkers in a cytokinesis structure is colocalization of each of the four biomarkers to the midbody.

In an embodiment the TGFβ receptor type 1 (TβR1) is the intracellular domain (TβR1-ICD). The term “TGFβ receptor type 1” may be used interchangeably with “TGFβ receptor type I” “TβR1”, “TGFβR1”, “TβRI” and “TGFβRI” herein.

Methods for assessing the presence and/or intracellular localization of biomarkers are well known in the art and any suitable method can be used. For example, the cytokinesis structure may be isolated and the presence of the biomarker in the cytokinesis structure assessed, or the cytokinesis structure may be identified by a detectable moiety and localization of a biomarker within that cytokinesis structure may be assessed by assessing whether the biomarker localizes to the same detectable moiety. Examples of techniques that can be used include those described herein and include immunohistochemistry, in situ hybridization, immunoprecipitation, immunofluorescence, confocal microscopy, many of which are exemplified in the Examples.

In some embodiments, diagnosing the cancer includes determining the malignancy of the cancer. In some embodiments, diagnosing the cancer includes determining the stage of the cancer. In some embodiments, diagnosing the cancer includes assessing the risk of cancer recurrence. In some embodiments, diagnosing the cancer includes assessing the grade of the cancer.

The invention also includes a method comprising the steps of:
providing a biological test sample from a subject;
determining the presence or absence of a first biomarker, a second biomarker, a third biomarker, and a fourth biomarker, the biomarkers are: Aurora kinase B (AURKB), Adaptor Protein, Phosphotyrosine Interacting With PH Domain And Leucine Zipper 1 (APPL1), TGFβ receptor type 1 (TβR1) and TNF receptor associated factor 6 (TRAF6), in the test sample; and
   wherein the co-localization of all four biomarkers in the biological test sample is indicative of cancer in the subject.

The invention also includes a method comprising the steps of:
providing a biological test sample from a subject;
determining the presence or absence of a first biomarker, a second biomarker, a third biomarker, and a fourth biomarker, the biomarkers are: Aurora kinase B (AURKB), Adaptor Protein, Phosphotyrosine Interacting With PH Domain And Leucine Zipper 1 (APPL1), TGFβ receptor type 1 (TβR1) and TNF receptor associated factor 6 (TRAF6), in the test sample; and
   wherein the presence of all four biomarkers co-localized to a cytokinesis structure in the biological test sample is indicative of cancer in the subject.

Thus, it will be appreciated that the second step may involve determining the co-localization of the biomarkers within the test sample.

The intracellular domain (ICD) of TβR1 is not cleaved off from the TβR1 in a healthy cell, thereby not detectable in the nucleus, which means that the three or four biomarkers (AURKB, APPL1, TβR1 (or TβR1-ICD) and TRAF6) co-localized during cytokinesis is not detectable in healthy cells.

Methods for determining the presence of biomarkers and/or whether biomarkers co-localize during cytokinesis and/or mitosis are known in the art. For example, to examine the immunofluorescence of proteins at each mitotic stage, cells can be synchronized (at the G1-S transition) by double-thymidine block and release, in order to enrich cytokinetic cells. A staging system can be used to identify the different phases of mitosis and cytokinesis based on the DNA and spindle morphology and extent of chromosome alignment and separation. Synchronization of mammalian cells in cytokinesis can also be achieved by releasing cells from pre-metaphase arrest. Pre-metaphase can achieved using microtubule synchronization be polymerizing/depolymerizing agents (such as nocodazole and taxol), as well as kinesin inhibitors (such as monastrol and S-trityl-L-cysteine).

It is a second object of the invention to provide a method for diagnosing and/or prognosing aggressive cancer in a subject, the method comprising the steps of:
a) providing a biological test sample from the subject;
b) determining the presence or absence of a first biomarker, a second biomarker, and a third biomarker, wherein said biomarkers are: Aurora kinase B (AURKB), Adaptor Protein, Phosphotyrosine Interacting With PH Domain And Leucine Zipper 1 (APPL1), and TGFβ receptor type 1 (TβR1), in said test sample; and
   wherein the co-localization of all three biomarkers in the biological sample is indicative of aggressive cancer in the subject.

It is a further object of the invention to provide a method for diagnosing and/or prognosing aggressive cancer in a subject, the method comprising the steps of:
a) providing a biological test sample from the subject;
b) determining the presence or absence of a first biomarker, a second biomarker, and a third biomarker, wherein said biomarkers are: Aurora kinase B (AURKB), Adaptor Protein, Phosphotyrosine Interacting With PH Domain And Leucine Zipper 1 (APPL1), and TGFβ receptor type 1 (TβR1), in said test sample; and
   wherein the presence of all three biomarkers co-localized to a cytokinesis structure in the biological sample is indicative of aggressive cancer in the subject.

An aggressive cancer form includes the meaning of high risk for metastasis. By aggressive cancer, we include a cancer comprising or consisting of stage III and/or stage IV cancer, for example as determined by the American Joint Committee on Cancer (AJCC) TNM system American Joint Committee on Cancer and the International Union Against Cancer.

Preferably, the cytokinesis structure is the midbody or the midzone of a cell.

In an embodiment the TGFβ receptor type 1 (TβR1) is the intracellular domain (TβR1-ICD).

Midbodies can be detected by using a molecule that binds to the midbody, such as a molecule that binds to a protein that is known to localize to the midbody, e.g., an antibody that specifically binds to a midbody polypeptide or an antigenic fragment thereof, e.g., Mitotic Kinesin-Like Protein-1 (MKLP-1), kinesin family member 4 (KIF4), and/or β-tubulin. MKLP-1 localizes to the spindle equator and is believed to participate in the separation of spindle poles during anaphase B of mitosis, by crosslinking antiparallel microtubules at the spindle midzone. A number of antibodies suitable for use in the methods described herein are known in the art and/or are commercially available. For example, anti-MKLP1 is available from BD Biosciences (San Jose, CA) and Santa Cruz Biotechnology Inc. (Santa Cruz, CA). Methods for isolating midbodies are known in the art (Science. 2004 Jul. 2; 305(5680): 61-66). Proteins present in the midbody preparations can then be identified by tandem liquid chromatography and tandem mass spectrometry.

In an embodiment, the method further comprises determining the presence or absence of a fourth biomarker in the biological test sample, wherein said biomarker is TNF receptor associated factor 6 (TRAF6), and wherein the co-localization of all four biomarkers in the biological test sample is indicative of aggressive cancer in the subject.

In an embodiment, the method further comprises determining the presence or absence of a fourth biomarker in the biological test sample, wherein said biomarker is TNF receptor associated factor 6 (TRAF6), wherein the presence of all four biomarkers co-localized to a cytokinesis structure in the biological test sample is indicative of aggressive cancer in the subject.

Further, the invention provides a method for diagnosing and/or prognosing aggressive cancer in a subject, the method comprising the steps of:
a) providing a biological test sample from the subject;
b) determining the presence or absence of a first biomarker, a second biomarker, a third biomarker, and a fourth biomarker, wherein said biomarkers are: Aurora kinase B (AURKB), Adaptor Protein, Phosphotyrosine Interacting With PH Domain And Leucine Zipper 1 (APPL1), and TGFβ receptor type 1 (TβR1) and TNF receptor associated factor 6 (TRAF6), in said test sample; and
   wherein the co-localization of all four biomarkers in the biological sample is indicative of aggressive cancer in the subject.

Further, the invention provides a method for diagnosing and/or prognosing aggressive cancer in a subject, the method comprising the steps of:
a) providing a biological test sample from the subject;
b) determining the presence or absence of a first biomarker, a second biomarker, a third biomarker, and a fourth biomarker, wherein said biomarkers are: Aurora kinase B (AURKB), Adaptor Protein, Phosphotyrosine Interacting With PH Domain And Leucine Zipper 1 (APPL1), and TGFβ receptor type 1 (TβR1) and TNF receptor associated factor 6 (TRAF6), in said test sample; and
   wherein the presence of all four biomarkers co-localized to a cytokinesis structure in the biological sample is indicative of aggressive cancer in the subject.

Preferably, AURKB is ubiquitinated. In an embodiment, the methods of the invention comprise detecting the presence of ubiquitinated AURKB in the biological test sample. Methods for detecting the presence of ubiquitinated AURKB are known in the art and disclosed herein.

Protein ubiquitination is a post-translational modification catalyzed by a cascade of enzymatic reactions involving a ubiquitin (Ub)-activating enzyme (E1), a Ub-conjugating enzyme (E2), and a Ub ligase (E3). Ub is conjugated onto protein substrates by formation of an isopeptide bond between the carboxyl group of the C-terminal glycine residue of Ub and the ϵ-amino group of a lysine residue in the substrate. Furthermore, a polyubiquitin (polyUb) chain is formed by conjugating the carboxyl group of the C-terminal glycine residue of Ub to the e-amino group of one of the seven internal lysines in the preceding Ub.

In other words, polyUbs are linked through the e-amino group of the Lys-48 and/or Lys-63 residues of the preceding Ub. In an embodiment, AURKB comprises the consensus sequence-(hydrophobic)-K-(hydrophobic)-K-X-(hydrophobic)-(polar)-(hydrophobic)-(polar)-(hydrophobic), in which at least one K is ubiquitinated. As shown in FIG. 3I, this motif is conserved in human, pig, cow, dog, mouse and rat AURKB. In an embodiment, AURKB comprises the *K*KX*&*&* consensus sequence, wherein *=hydrophobic, &=polar, X=any amino acid, K=acceptor lysine, and at least one of the lysine residues therein is ubiquitinated. In an embodiment, AURKB comprises the GKGKFGNVYL (SEQ ID NO: 23) consensus sequence and at least one of the lysine residues therein is ubiquitinated. In other words, in an embodiment AURKB is ubiquitinated at one or both lysine residues corresponding to Lysine 85 (K85) and/or Lysine 87 (K87) of human AURKB (SEQ ID NO: 1). In an embodiment, AURKB is ubiquitinated at a lysine residue corresponding to Lysine 85 (K85) of human AURKB (SEQ ID NO: 1). In an embodiment, AURKB is ubiquitinated at a lysine residue corresponding to Lysine 87 (K87) of human AURKB (SEQ ID NO: 1). In an embodiment, AURKB is ubiquitinated at both lysine residues corresponding to Lysine 85 (K85) and Lysine 87 (K87) of human AURKB (SEQ ID NO: 1).

By “corresponding to” we include the meaning of the lysine residue in another AURKB (such as an orthologue or variant of human AURKB) which aligns to K85 in human AURKB (SEQ ID NO: 1 and/or to K87 in human AURKB (SEQ ID NO: 1) when the sequence of human AURKB and the sequence of a different AURKB are compared, such as are aligned using MacVector, ClustalOmega, or ClustalW2, or are aligned as shown in FIG. 1 of Brown et al., Evolutionary Biology volume 4, Article number: 39 (2004), incorporated by reference.

| SEQ ID NO | Sequence | |
|---|---|---|
| AURKB amino | 1 | maqkensypw pygrqtapsg lstlpqrvlr kepvtpsalv |
| acid sequence | | lmsrsnvqpt aapgqkvmen |
| (SEQ ID NO: 1) | 61 | ssgtpdiltr hftiddfeig rplgkgkfgn vylarekksh |
| | | fivalkvlfk sqiekegveh |
| | 121 | qlrreieiqa hlhhpnilrl ynyfydrrri ylileyaprg |
| | | elykelqksc tfdeqrtati |
| | 181 | meeladalmy chgkkvihrd ikpenlllgl kgelkiadfg |
| | | wsvhapslrr ktmcgtldyl |
| | 241 | ppemiegrmh nekvdlwcig vlcyellvgn ppfesashne |
| | | tyrrivkvdl kfpasvpmga |
| | 301 | qdliskllrh npserlplaq vsahpwvran srrvlppsal |
| | | qsva |

| | |
|---|---|
| AURKB coding | ATGGCCCAGAAGGAGAACTCCTACCCCTGGCCCTACGGCCGACAGACGG |
| sequence | CTCCATCTGGCCTGAGCACCCTGCCCCAGCGAGTCCTCCGGAAAGAGCC |
| (SEQ ID NO: 2) | TGTCACCCCATCTGCACTTGTCCTCATGAGCCGCTCCAATGTCCAGCCC |
| | ACAGCTGCCCCTGGCCAGAAGGTGATGGAGAATAGCAGTGGGACACCCG |
| | ACATCTTAACGCGGCACTTCACAATTGATGACTTTGAGATTGGGCGTCC |
| | TCTGGGCAAAGGCAAGTTTGGAAACGTGTACTTGGCTCGGGAGAAGAAA |
| | AGCCATTTCATCGTGGCGCTCAAGGTCCTCTTCAAGTCCCAGATAGAGA |
| | AGGAGGGCGTGGAGCATCAGCTGCGCAGAGAGATCGAAATCCAGGCCCA |
| | CCTGCACCATCCCAACATCCTGCGTCTCTACAACTATTTTTATGACCGG |
| | AGGAGGATCTACTTGATTCTAGAGTATGCCCCCCGCGGGGAGCTCTACA |
| | AGGAGCTGCAGAAGAGCTGCACATTTGACGAGCAGCGAACAGCCACGAT |
| | CATGGAGGAGTTGGCAGATGCTCTAATGTACTGCCATGGGAAGAAGGTG |
| | ATTCACAGAGACATAAAGCCAGAAAATCTGCTCTTAGGGCTCAAGGGAG |
| | AGCTGAAGATTGCTGACTTCGGCTGGTCTGTGCATGCGCCCTCCCTGAG |
| | GAGGAAGACAATGTGTGGCACCCTGGACTACCTGCCCCCAGAGATGATT |
| | GAGGGGCGCATGCACAATGAGAAGGTGGATCTGTGGTGCATTGGAGTGC |
| | TTTGCTATGAGCTGCTGGTGGGGAACCCACCCTTTGAGAGTGCATCACA |
| | CAACGAGACCTATCGCCGCATCGTCAAGGTGGACCTAAAGTTCCCCGCT |
| | TCCGTGCCCATGGGAGCCCAGGACCTCATCTCCAAACTGCTCAGGCATA |
| | ACCCCTCGGAACGGCTGCCCCTGGCCCAGGTCTCAGCCCACCCTTGGGT |
| | CCGGGCCAACTCTCGGAGGGTGCTGCCTCCCTCTGCCCTTCAATCTGTC |
| | GCCTGA |

In an embodiment, AURKB is Lys48-linked and/or Lys63-linked polyubiquitinated.

In the accompanying Examples, the inventors surprisingly found that AURKB contains at least one acceptor lysine residue that serves as the recognition site for ubiquitination by TRAF6, and that TRAF6-mediated ubiquitination of AURKB on K85 and/or K87 in the consensus sequence contributes to its activity and controls the localization of TβRI in the midbody during cell division. Methods for determining whether a protein is ubiquitinated are known in the art and include an in vivo ubiquitination assay, or an in situ PLA assay with two antibodies (AURKB and K63 antibodies) as described in the Examples.

The method(s) disclosed in the present specification is/are suitable for cancer types associated with and/or mediated by proteolytic cleavage of transforming growth factor β type I receptor (TβRI).

By a cancer “associated with and/or mediated by the proteolytic cleavage of transforming growth factor β type I receptor (TβRI)” we include the meaning of a cancer in which the intracellular domain (ICD) of TβRI has been proteolytically cleaved and enters the nucleus to promote transcription of pro-invasive genes. Methods of detecting the localization of TβRI and TβRI-ICD are described herein.

The cancer is for example a solid tumour. The tumour may be selected from the group consisting of prostate cancer, renal carcinoma, lung cancer, kidney cancer, gastric cancer, bladder carcinoma, breast cancer, endometrial cancer, ovarian cancer, and colorectal cancer.

Preferably, the cancer is prostate cancer. In a further embodiment, the prostate cancer is castration-resistant prostate cancer (CRPC). By “castration resistant prostate cancer (CRPC)” we include the meaning of a form of prostate cancer wherein the cancer is no longer stopped by low testosterone levels (less than 50 ng/ml). Castration-resistant prostate cancer is defined by a rising PSA level and/or worsening symptoms and/or growing cancer verified by scans. In an embodiment, the CRPC is of the neuroendocrine type. In an embodiment, the biological test sample comprises CRPC cells. As shown in the accompanying Examples, the inventors surprisingly found that during mitosis and cytokinesis, a TβRI-AURKB complex was formed in midbody in CRPC cells and neuroblastoma KELLY cells.

Preferably, the biological test sample is a tissue sample, such as a biopsy from a tumour.

The “sample to be tested”, “biological test sample”, “test sample” or “control sample” may be a tissue or fluid sample taken or derived from a subject.

Preferably the test sample is provided from a mammal. The mammal may be any domestic or farm animal. Preferably, the mammal is a rat, mouse, guinea pig, cat, dog, horse or a primate. Most preferably, the mammal is human.

A sample as used herein includes any relevant biological sample that can be used for molecular profiling, e.g., sections of tissues such as biopsy or tissue removed during surgical or other procedures, bodily fluids (e.g. liquid biopsy), autopsy samples, and frozen sections taken for histological purposes, a sample comprising cells. Such samples include blood or blood fractions or products (e.g. serum, buffy coat, plasma, platelets, red blood cells, and the like), sputum, malignant effusion, cheek cells tissue, cultured cells (e.g., primary cultures, explants, and transformed cells), stool, urine, other biological or bodily fluids (e.g., prostatic fluid, gastric fluid, intestinal fluid, renal fluid, lung fluid, cerebrospinal fluid, and the like), etc. The sample can comprise biological material that is a fresh frozen & formalin fixed paraffin embedded (FFPE) block, formalin-fixed paraffin embedded, or is within an RNA preservative and formalin fixative. More than one sample of more than one type can be used for each subject. Preferably the sample is a cell or tissue sample (or derivative thereof), for example one comprising or consisting of cancer cells. In a preferred embodiment, the sample comprises a fixed tumor sample. The sample used in the methods described herein can be a formalin fixed paraffin embedded (FFPE) sample. The FFPE sample can be one or more of fixed tissue, unstained slides, bone marrow core or clot, core needle biopsy, malignant fluids and fine needle aspirate (FNA). In an embodiment, the fixed tissue comprises a tumor containing formalin fixed paraffin embedded (FFPE) block from a surgery or biopsy.

A sample may be processed according to techniques understood by those in the art. A sample can be without limitation fresh, frozen or fixed cells or tissue. In some embodiments, a sample comprises formalin-fixed paraffin-embedded (FFPE) tissue, fresh tissue or fresh frozen (FF) tissue. A sample can comprise cultured cells, including primary or immortalized cell lines derived from a sample from a subject. A sample can also refer to an extract from a sample from a subject. For example, a sample can comprise DNA, RNA or protein extracted from a tissue or a bodily fluid. Many techniques and commercial kits are available for such purposes. The fresh sample from the subject can be treated with an agent to preserve RNA prior to further processing, e.g., cell lysis and extraction. Samples can include frozen samples collected for other purposes. Samples can be associated with relevant information such as age, gender, and clinical symptoms present in the subject; source of the sample; and methods of collection and storage of the sample.

A biopsy comprises the process of removing a tissue sample for diagnostic or prognostic evaluation, and to the tissue specimen itself. Any biopsy technique known in the art can be applied to the methods of the present invention. The biopsy technique applied can depend on the tissue type to be evaluated (e.g., colon, prostate, kidney, bladder, lymph node, liver, bone marrow, blood cell, lung, breast, etc.), the size and type of the tumor (e.g., solid or suspended, blood or ascites), among other factors. Representative biopsy techniques include, but are not limited to, excisional biopsy, incisional biopsy, needle biopsy, surgical biopsy, and bone marrow biopsy. An “excisional biopsy” refers to the removal of an entire tumor mass with a small margin of normal tissue surrounding it. An “incisional biopsy” refers to the removal of a wedge of tissue that includes a cross-sectional diameter of the tumor. The method may use a “core-needle biopsy” of the tumor mass, or a “fine-needle aspiration biopsy” which generally obtains a suspension of cells from within the tumor mass. Biopsy techniques are discussed, for example, in Harrison's Principles of Internal Medicine, Kasper, et al., eds., 16th ed., 2005, Chapter 70, and throughout Part V.

Preferably test and control samples are derived from the same species. Preferably test and control samples are matched for age, gender and/or lifestyle.

In an embodiment the tissue sample is tumour tissue, such as a biopsy. In an embodiment, the cell sample is a sample of cancer cells.

Preferably, the method further comprises the steps of:
c) providing one or more control sample from:
   i. an individual not afflicted with cancer; and/or
   ii. an individual afflicted with cancer, wherein the control sample is of a different stage of cancer to that of the test sample, or wherein the control sample is derived from healthy tissue from an individual afflicted with cancer;
d) determining the presence or absence of a first biomarker, a second biomarker, and a third biomarker, wherein said biomarkers are: Aurora kinase B (AURKB), Adaptor Protein, Phosphotyrosine Interacting With PH Domain And Leucine Zipper 1 (APPL1), and TGFβ receptor type 1 (TβR1), in the control sample;
   wherein cancer is diagnosed in the event that all three biomarkers measured in step (b) are co-localized in the test sample, and not all three biomarkers measured in step (d) are co-localized in the control sample.

Preferably, the method further comprises the steps of:
c) providing one or more control sample from:
   i. an individual not afflicted with cancer; and/or
   ii. an individual afflicted with cancer, wherein the control sample was of a different stage of cancer to that of the test sample, or wherein the control sample is derived from healthy tissue from an individual afflicted with cancer;
d) determining the presence or absence of a first biomarker, a second biomarker, and a third biomarker, wherein said biomarkers are: Aurora kinase B (AURKB), Adaptor Protein, Phosphotyrosine Interacting With PH Domain And Leucine Zipper 1 (APPL1), and TGFβ receptor type 1 (TβR1), in the control sample;
   wherein cancer is diagnosed in the event that all three biomarkers measured in step (b) are co-localized to a cytokinesis structure in the test sample, and not all three biomarkers measured in step (d) are co-localized to a cytokinesis structure in the control sample.

For example, if the cancer is strictly localized to one lobe of the prostate it may be possible to use healthy (i.e. non-cancerous) tissue in another lobe from the same individual as control.

In an embodiment, the method further comprises (d) determining the presence or absence of a fourth biomarker in the control sample, wherein said biomarker is TNF receptor associated factor 6 (TRAF6), wherein cancer is diagnosed in the event that all four biomarkers measured in step (b) are co-localized in the test sample, and not all four biomarkers measured in step (d) are co-localized in the control sample.

In an embodiment, the method further comprises (d) determining the presence or absence of a fourth biomarker in the control sample, wherein said biomarker is TNF receptor associated factor 6 (TRAF6), wherein cancer is diagnosed in the event that all four biomarkers measured in step (b) are co-localized to a cytokinesis structure in the test sample, and not all four biomarkers measured in step (d) are co-localized to a cytokinesis structure in the control sample.

Thus, preferably, the method further comprises the steps of:
c) providing one or more control sample from:
   i. an individual not afflicted with cancer; and/or
   ii. an individual afflicted with cancer, wherein the control sample is of a different stage of cancer to that of the test sample;

d) determining the presence or absence of a first biomarker, a second biomarker, a third biomarker, and a fourth biomarker, wherein said biomarkers are: Aurora kinase B (AURKB), Adaptor Protein, Phosphotyrosine Interacting With PH Domain And Leucine Zipper 1 (APPL1), TGFβ receptor type 1 (TβR1) and TNF receptor associated factor 6 (TRAF6), in the control sample;

wherein cancer is diagnosed in the event that all four biomarkers measured in step (b) are co-localized in the test sample, and not all four biomarkers measured in step (d) are co-localized in the control sample.

Thus, preferably, the method further comprises the steps of:
c) providing one or more control sample from:
   i. an individual not afflicted with cancer; and/or
   ii. an individual afflicted with cancer, wherein the control sample was of a different stage of cancer to that of that the test sample;
d) determining the presence or absence of a first biomarker, a second biomarker, a third biomarker, and a fourth biomarker, wherein said biomarkers are: Aurora kinase B (AURKB), Adaptor Protein, Phosphotyrosine Interacting With PH Domain And Leucine Zipper 1 (APPL1), TGFβ receptor type 1 (TβR1) and TNF receptor associated factor 6 (TRAF6), in the control sample;
   wherein cancer is diagnosed in the event that all four biomarkers measured in step (b) are co-localized to a cytokinesis structure in the test sample, and not all four biomarkers measured in step (d) are co-localized to a cytokinesis structure in the control sample.

Preferably, the AURKB is ubiquitinated.

By “wherein the control sample was of a different stage of cancer to that of that the test sample” we include the meaning that the control sample is derived from an individual afflicted with cancer, but the cancer comprised within the control sample is less advanced (i.e. lower grade or score) than the cancer in the test sample. The cancer may be diagnosed in the individual afflicted with cancer using conventional clinical methods known in the art.

By “wherein the control sample is derived from healthy tissue from an individual afflicted with cancer”, we include the meaning that the control sample may be derived from healthy, non-cancerous tissue that is adjacent to the cancerous tissue.

As exemplified in the accompanying examples, the presence of Aurora kinase B (AURKB), Adaptor Protein, Phosphotyrosine Interacting With PH Domain And Leucine Zipper 1 (APPL1), TGFβ receptor type 1 (TβR1) and TNF receptor associated factor 6 (TRAF6) in a cytokinesis structure is indicative of cancer in a subject.

Preferably, the individual not afflicted with cancer was not, at the time the sample was obtained, afflicted with any disease or condition. Preferably, the individual not afflicted with cancer is a healthy individual.

Preferably, the presence or absence of biomarkers Aurora kinase B (AURKB), Adaptor Protein, Phosphotyrosine Interacting With PH Domain And Leucine Zipper 1 (APPL1), TGFβ receptor type 1 (TβR1) and/or TNF receptor associated factor 6 (TRAF6), preferably co-localized to a cellular structure such as a cytokinesis structure, is determined by detecting the biomarker protein; and/or detecting a biological activity of the biomarker protein.

In an embodiment the TGFβ receptor type 1 (TβR1) is the intracellular domain (TβR1-ICD).

By detecting the biomarker protein we include the meaning of detecting whether the biomarker protein is present directly, for example by using a binding partner that specifically binds to the biomarker protein. By detecting a biological activity of the biomarker protein we include the meaning of assaying for a biological activity of the biomarker protein, for example an enzymatic activity. It will be appreciated that detecting a biological activity of the biomarker protein may be used to indirectly determine the presence or absence of the biomarker.

The presence and/or absence of said biomarkers, preferably co-localized to a cellular structure such as a cytokinesis structure may be determined by immunohistochemistry, immunocytochemistry, immunoprecipitation (IP), ELISA techniques (single or mulitplex), radioimmunoassay (RIA), immunoradiometric assays (IRMA) and immunoenzymatic assays (IEMA), including sandwich assays using monoclonal and/or polyclonal antibodies, in situ proximity ligation assay (PLA), enzymatic methods, image analysis, mass spectrometry, aptamers, Bio-Layer Interferometry (BLI), Surface plasmon resoncance (SPR), Multiplex assay (MSD, Mesoscale discovery), or by indicator substances that bind to Aurora kinase B (AURKB), Adaptor Protein, Phosphotyrosine Interacting With PH Domain And Leucine Zipper 1 (APPL1), TGFβ receptor type 1 intracellular domain (TβR1-ICD) and TNF receptor associated factor 6 (TRAF6).

Immunohistochemistry (IHC) is a process of localizing antigens (e.g., proteins) in cells of a tissue binding antibodies specifically to antigens in the tissues. The antigen-binding antibody can be conjugated or fused to a tag that allows its detection, e.g., via visualization. In some embodiments, the tag is an enzyme that can catalyze a color-producing reaction, such as alkaline phosphatase or horseradish peroxidase. The enzyme can be fused to the antibody or non-covalently bound, e.g., using a biotin-avadin system. Alternatively, the antibody can be tagged with a fluorophore, such as fluorescein, rhodamine, DyLight Fluor or Alexa Fluor. The antigen-binding antibody can be directly tagged or it can itself be recognized by a detection antibody that carries the tag. Using IHC, one or more proteins may be detected. The expression of a gene product can be related to its staining intensity compared to control levels. In some embodiments, the gene product is considered differentially expressed if its staining varies at least 1.2, 1.3, 1.4, 1.5, 1.6, 1.7, 1.8, 1.9, 2.0, 2.2, 2.5, 2.7, 3.0, 4, 5, 6, 7, 8, 9 or 10-fold in the sample versus the control.

IHC comprises the application of antigen-antibody interactions to histochemical techniques. In an illustrative example, a tissue section is mounted on a slide and is incubated with antibodies (polyclonal or monoclonal) specific to the antigen (primary reaction). The antigen-antibody signal is then amplified using a second antibody conjugated to a complex of peroxidase antiperoxidase (PAP), avidin-biotin-peroxidase (ABC) or avidin-biotin alkaline phosphatase. In the presence of substrate and chromogen, the enzyme forms a colored deposit at the sites of antibody-antigen binding.

Immunofluorescence is an alternate approach to visualize target proteins. In this technique, the primary target-antibody signal is amplified using a second antibody conjugated to a fluorochrome. On UV light absorption, the fluorochrome emits its own light at a longer wavelength (fluorescence), thus allowing localization of antibody-antigen complexes.

Protein-based techniques for detecting the presence and/or amount of a biomarker also include immunoaffinity assays based on antibodies selectively immunoreactive for the protein encoding the biomarker. These techniques include without limitation immunoprecipitation, Western blot analysis, molecular binding assays, enzyme-linked immunosorbent assay (ELISA), enzyme-linked immunofiltration assay (ELIFA), fluorescence activated cell sorting (FACS) and the like. For example, an optional method of detecting the presence and/or absence of a biomarker in a sample comprises contacting the sample with an antibody against the biomarker, or an immunoreactive fragment of the antibody thereof, or a recombinant protein containing an antigen binding region of an antibody against the biomarker under conditions sufficient for an antibody-biomarker complex to form; and then detecting said complex. Methods for producing such antibodies are known in the art. ELISA methods are well known in the art, for example see The ELISA Guidebook (Methods in Molecular Biology), 2000, Crowther, Humana Press, ISBN-13: 978-0896037281 (the disclosures of which are incorporated by reference. A wide range of immunoassay techniques using such an assay format are available, see, e.g., U.S. Pat. Nos. 4,016,043, 4,424,279 and 4,018,653. These include both single-site and two-site or “sandwich” assays of the non-competitive types, as well as in the traditional competitive binding assays. These assays also include direct binding of a labelled antibody to a target biomarker. Suitable binding agents (also referred to as binding molecules) can be selected from a library, based on their ability to bind a given protein.

Antibodies can be used to immunoprecipitate specific proteins from solution samples or to immunoblot proteins separated by, e.g., polyacrylamide gel electrophoresis.

Preferably, step (b) and/or (d) is performed by labelling the one or more biomarkers in the test sample(s) with a detectable moiety.

Preferably, step (b) and/or (d) is performed by labelling the one or more biomarkers in the control sample(s) with a detectable moiety.

By a “detectable moiety” we include the meaning that the moiety is one which may be detected, such as visualized, qualified as being present or not, and/or quantitated. By a moiety being detectable, the relative amount and/or location of the moiety may be determined. Suitable detectable moieties are well known in the art.

Thus, the detectable moiety may be a fluorescent and/or luminescent and/or chemiluminescent moiety which, when exposed to specific conditions, may be detected. For example, a fluorescent moiety may need to be exposed to radiation (i.e. light) at a specific wavelength and intensity to cause excitation of the fluorescent moiety, thereby enabling it to emit detectable fluorescence at a specific wavelength that may be detected.

Alternatively, the detectable moiety may be an enzyme which is capable of converting a (preferably undetectable) substrate into a detectable product that can be visualized and/or detected. Examples of suitable enzymes are discussed in more detail below in relation to, for example, ELISA assays.

Alternatively, the detectable moiety may be a radioactive atom which is useful in imaging. Suitable radioactive atoms include 99mTc and 123I for scintigraphic studies. Other readily detectable moieties include, for example, spin labels for magnetic resonance imaging (MRI) such as 123I again, 131I, 111In, 19F, 13C, 15N, 17O, gadolinium, manganese or iron. Clearly, the agent to be detected (such as, for example, biomarkers in the test sample and/or control sample described herein and/or an antibody molecule for use in detecting a selected protein) must have sufficient of the appropriate atomic isotopes in order for the detectable moiety to be readily detectable.

The radio- or other labels may be incorporated into the agents of the invention (i.e. the proteins present in the samples of the methods of the invention and/or the binding agents of the invention) in known ways. For example, if the binding moiety is a polypeptide it may be biosynthesized or may be synthesized by chemical amino acid synthesis using suitable amino acid precursors involving, for example, fluorine-19 in place of hydrogen. Labels such as 99mTc, 123I, 186Rh, 188Rh and 111In can, for example, be attached via cysteine residues in the binding moiety. Yttrium-90 can be attached via a lysine residue. The IODOGEN method (Fraker et al (1978) Biochem. Biophys. Res. Comm. 80, 49-57) can be used to incorporate 123I. Reference (“Monoclonal Antibodies in Immunoscintigraphy”, J-F Chatal, CRC Press, 1989) describes other methods in detail. Methods for conjugating other detectable moieties (such as enzymatic, fluorescent, luminescent, chemiluminescent or radioactive moieties) to proteins are well known in the art.

Preferably, step (b) and/or (d) is performed using one or more first binding agent capable of binding to said biomarker. It will be appreciated by persons skilled in the art that the first binding agent may comprise or consist of a single species with specificity for one of the biomarkers or a plurality of different species, each with specificity for a different protein biomarker.

Preferably, step (b) and/or (d) is performed using an assay comprising a second binding agent capable of binding to said first binding agent, the second binding agent comprising a detectable moiety.

At least one type of the binding agents, and more typically all of the types, may comprise or consist of an antibody or antigen-binding fragment of the same, or a variant thereof.

Preferably, the first binding agent and/or the second binding agent comprises or consists of an antibody or an antigen-binding fragment thereof.

The antibody or antigen binding fragment thereof may be a scFv; Fab; or a binding domain of an immunoglobulin molecule.

Preferably, the detectable moiety is selected from the group consisting of: a fluorescent moiety; a luminescent moiety; a chemiluminescent moiety; a radioactive moiety; an enzymatic moiety.

In yet another embodiment the presence and/or absence of Aurora kinase B (AURKB), Adaptor Protein, Phosphotyrosine Interacting With PH Domain And Leucine Zipper 1 (APPL1), TGFβ receptor type 1 (TβR1) and/or TNF receptor associated factor 6 (TRAF6) is determined by measuring the presence and/or expression of a nucleic acid molecule encoding the biomarker.

Preferably, the nucleic acid molecule is a cDNA molecule or an mRNA molecule.

Any method of detecting and/or quantitating the nucleic acid molecule encoding the biomarker can in principle be used to determine the presence and/or absence of the biomarker. The nucleic acid molecule encoding the biomarker can be directly detected and/or quantitated (such as by RNA sequencing), or may be copied and/or amplified to allow detection of amplified copies of the nucleic acid molecule encoding the biomarker or its complement.

Preferably, determining the presence and/or absence of the biomarkers in step (b), (d) and/or (f) is performed using a method selected from the group consisting of Southern hybridization, Northern hybridization, polymerase chain reaction (PCR), reverse transcriptase PCR (RT PCR), quantitative real-time PCR (qRT-PCR), nanoarray, microarray, macroarray, autoradiography and in situ hybridization.

Reverse transcription can be performed by any method known in the art. For example, reverse transcription may be performed using the Omniscript kit (Qiagen, Valencia, CA), Superscript III kit (Invitrogen, Carlsbad, CA), for RT-PCR. Target-specific priming can be performed in order to increase the sensitivity of detection of target sequences and generate target-specific cDNA. RT-PCR can be performed using for example Applied Biosystems Prism (ABI) 7900 HT instruments, or Thermo Fisher QuantStudio Real Time PCR instruments or any other thermocycler with fluorescent real time detection of the amplification, in a volume with target sequence-specific cDNA or messenger RNA equivalent to 1 ng total RNA or more. Primers and probes concentrations for TaqMan® analysis are added to amplify fluorescent amplicons using PCR cycling conditions such as 95° C. for 10 minutes for one cycle, 95° C. for 20 seconds, and 60° C. for 45 seconds for 40 cycles. The amplification reaction can also be performed as a one-step qRT-PCR using either one single thermostable DNA polymerase capable of performing both the reverse transcription and the DNA polymerisation such as the Tth Polymerase originally isolated from Thermus thermophilus. It is also feasible to perform a one-step qPCR with a mixture of reverse transcriptase and thermostable DNA polymerase. PCR products can also be labelled with a fluorescent dye, such as SYBR Green or any other fluorescent dye detected by the instrument.

The amplification can be designed to determine the presence and/or absence of all the biomarkers in step (b), (d) and/or (f) either as single entities or in combination such as in multiplex PCR or digital PCR (dPCR) A reference sample can be assayed to ensure reagent and process stability. The reference sample can be obtained from a cell line expressing the target messenger RNA or be obtained as synthetized messenger RNA. A reference sample can be assayed to ensure reagent and process stability. Negative controls (e.g., no template) should be assayed to monitor any exogenous nucleic acid contamination.

In situ hybridization assays are well known and are generally described in Angerer et al., Methods Enzymol. 152:649-660 (1987). In an in situ hybridization assay, cells, e.g., from a biopsy, are fixed to a solid support, typically a glass slide. If DNA is to be probed, the cells are denatured with heat or alkali. The cells are then contacted with a hybridization solution at a moderate temperature to permit annealing of specific probes that are labeled. The probes are preferably labeled, e.g., with radioisotopes or fluorescent reporters, or enzymatically. FISH (fluorescence in situ hybridization) uses fluorescent probes that bind to only those parts of a sequence with which they show a high degree of sequence similarity. CISH (chromogenic in situ hybridization) uses conventional peroxidase or alkaline phosphatase reactions visualized under a standard bright-field microscope.

In situ hybridization can be used to detect specific gene sequences in tissue sections or cell preparations by hybridizing the complementary strand of a nucleotide probe to the sequence of interest. Fluorescent in situ hybridization (FISH) uses a fluorescent probe to increase the sensitivity of in situ hybridization.

FISH is a cytogenetic technique used to detect and localize specific polynucleotide sequences in cells. For example, FISH can be used to detect DNA sequences on chromosomes. FISH can also be used to detect and localize specific RNAs, e.g., mRNAs, within tissue samples. In FISH uses fluorescent probes that bind to specific nucleotide sequences to which they show a high degree of sequence similarity. Fluorescence microscopy can be used to find out whether and where the fluorescent probes are bound. In addition to detecting specific nucleotide sequences, e.g., translocations, fusion, breaks, duplications and other chromosomal abnormalities, FISH can help define the spatial-temporal patterns of specific gene copy number and/or gene expression within cells and tissues.

In an embodiment, determining the presence and/or absence of the biomarkers in step (b) and/or (d) is performed using one or more binding moieties, each individually capable of binding selectively to a nucleic acid molecule encoding one of the biomarkers. Preferably, the one or more binding moieties each comprise or consist of a nucleic acid molecule.

Preferably, the one or more binding moieties each comprise or consist of DNA, RNA, PNA, LNA, GNA, TNA or PMO.

Preferably, the one or more binding moieties comprises a detectable moiety. Preferably, the detectable moiety is selected from the group consisting of: a fluorescent moiety; a luminescent moiety; a chemiluminescent moiety; a radioactive moiety (for example, a radioactive atom); or an enzymatic moiety.

The radioactive atom may be technetium-99m, iodine-123, iodine 125, iodine-131, indium-111, fluorine-19, carbon-13, nitrogen-15, oxygen-17, phosphorus-32, sulphur-35, deuterium, tritium, rhenium-186, rhenium-188 and yttrium-90.

Preferably, the detectable moiety of the binding moiety is a fluorescent moiety

It is a further object of the invention to provide a method for diagnosing cancer in a subject comprising the steps of:
a) providing a biological test sample from a subject; and
b) determining the presence and/or amount of a first biomarker, a second biomarker, and a third biomarker, wherein said biomarkers are: Aurora kinase B (AURKB), Adaptor Protein, Phosphotyrosine Interacting With PH Domain And Leucine Zipper 1 (APPL1), and TGFβ receptor type 1 (TβR1),
c) providing one or more control sample from:
   i. an individual not afflicted with cancer; and/or
   ii. an individual afflicted with cancer, wherein the control sample was of a different stage of cancer to that of that the test sample, or wherein the control sample is derived from healthy tissue from an individual afflicted with cancer;
d) determining the presence and/or amount of a first biomarker, a second biomarker, and a third biomarker, wherein said biomarkers are: Aurora kinase B (AURKB), Adaptor Protein, Phosphotyrosine Interacting With PH Domain And Leucine Zipper 1 (APPL1), and TGFβ receptor type 1 (TβR1) in the control sample;
   wherein cancer is diagnosed in the event that all three biomarkers are present in the test sample, and not all three biomarkers are present in the control sample; and/or wherein the cancer is diagnosed in the event that the amount of the three biomarkers in the test sample in step (b) is increased relative to the amount of the three biomarkers in the control sample measured in step (d).

It is a further object of the invention to provide a method for diagnosing cancer in a subject comprising the steps of:
a) providing a biological test sample from a subject; and
b) determining the presence and/or amount of a first biomarker, a second biomarker, a third biomarker, and a fourth biomarker, wherein said biomarkers are: Aurora kinase B (AURKB), Adaptor Protein, Phosphotyrosine Interacting With PH Domain And Leucine Zipper 1 (APPL1), TGFβ receptor type 1 (TβR1) and TNF receptor associated factor 6 (TRAF6),
c) providing one or more control sample from:
   i. an individual not afflicted with cancer; and/or
   ii. an individual afflicted with cancer, wherein the control sample was of a different stage of cancer to that of that the test sample, or wherein the control sample is derived from healthy tissue from an individual afflicted with cancer;
d) determining the presence and/or amount of a first biomarker, a second biomarker, a third biomarker, and a fourth biomarker, wherein said biomarkers are: Aurora kinase B (AURKB), Adaptor Protein, Phosphotyrosine Interacting With PH Domain And Leucine Zipper 1 (APPL1), TGFβ receptor type 1 (TβR1) and TNF receptor associated factor 6 (TRAF6), in the control sample; wherein cancer is diagnosed in the event that all four biomarkers are present in the test sample, and not all four biomarkers are present in the control sample; and/or
   wherein the cancer is diagnosed in the event that the amount of the four biomarkers in the test sample in step (b) is increased relative to the amount of the four biomarkers in the control sample measured in step (d).

Preferably, the cancer is prostate cancer. In a further embodiment, the prostate cancer is castration-resistant prostate cancer (CRPC). In an embodiment, the CRPC is of the neuroendocrine type.

This method of the invention comprises expression profiling, which includes assessing differential expression of the biomarkers disclosed herein. Differential expression can include overexpression and/or underexpression of a biological product, e.g., a gene, mRNA or protein, compared to a control (or a reference). Determining the presence and/or amount of said biomarkers can be performed by any of the proteins or nucleic acid-based techniques described herein. The control sample can include similar cells to the test sample but without the disease (e.g., expression profiles obtained from samples from healthy individuals). A control can be a previously determined level that is indicative of a drug target efficacy associated with the particular disease and the particular drug target. The control can be derived from the same subject, e.g., a normal adjacent portion of the same organ as the diseased cells, the control can be derived from healthy tissues (i.e. non-cancerous tissues) from other individuals, or previously determined thresholds that are indicative of a disease responding or not-responding to a particular drug target. The control can also be a control found in the same sample, e.g. a housekeeping gene or a product thereof (e.g., mRNA or protein). For example, a control nucleic acid can be one which is known not to differ depending on the cancerous or non-cancerous state of the cell. The expression level of a control nucleic acid can be used to normalize signal levels in the test and reference populations. Illustrative control genes include, but are not limited to, e.g., β-actin, glyceraldehyde 3-phosphate dehydrogenase and ribosomal protein P1. Multiple controls or types of controls can be used. The source of differential expression can vary. For example, a gene copy number may be increased in a cell, thereby resulting in increased expression of the gene. Alternately, transcription of the gene may be modified, e.g., by chromatin remodeling, differential methylation, changes in promoter or enhancer regions, differential expression or activity of transcription factors, etc. Translation may also be modified, e.g., by differential expression of factors that degrade mRNA, translate mRNA, or silence translation, e.g., microRNAs or siRNAs or changes due to alternative splicing. In some embodiments, differential expression comprises differential activity. For example, a protein may carry a mutation that increases the activity of the protein, such as constitutive activation, thereby contributing to a diseased state. Molecular profiling that reveals changes in activity can be used to guide treatment selection.

The level of expression of Aurora kinase B (AURKB), Adaptor Protein, Phosphotyrosine Interacting With PH Domain And Leucine Zipper 1 (APPL1), TGFβ receptor type 1 (TβR1) and/or TNF receptor associated factor 6 (TRAF6) may be determined by measuring DNA, mRNA or cDNAs coding for said respective biomarker (Aurora kinase B (AURKB), Adaptor Protein, Phosphotyrosine Interacting With PH Domain And Leucine Zipper 1 (APPL1), TGFβ receptor type 1 intracellular domain (TβR1-ICD) and TNF receptor associated factor 6 (TRAF6)) and/or fragments thereof.

In the context of the present invention, an increased level of said biomarkers: Aurora kinase B (AURKB), Adaptor Protein, Phosphotyrosine Interacting With PH Domain And Leucine Zipper 1 (APPL1), TGFβ receptor type 1 (TβR1) and TNF receptor associated factor 6 (TRAF6) in the test sample compared to the level of biomarkers in the control sample is indicative of cancer in the subject. For example, when the level of Aurora kinase B (AURKB) is increased in the test sample relative to the level of Aurora kinase B (AURKB) in the control sample, when the level of Adaptor Protein, Phosphotyrosine Interacting With PH Domain And Leucine Zipper 1 (APPL1) is increased in the test sample relative to the level of Adaptor Protein, Phosphotyrosine Interacting With PH Domain And Leucine Zipper 1 (APPL1) in the control sample, when the level TGFβ receptor type 1 (TβR1) is increased in the test sample relative to the level of TGFβ receptor type 1 (TβR1) in the control sample, and when the level TNF receptor associated factor 6 (TRAF6) is increased in the test sample relative to the level of TNF receptor associated factor 6 (TRAF6) in the control sample, the test sample is indicative of cancer in the subject.

By “is increased relative to the amount in a control sample” we include the meaning of the amount of the biomarkers in the test sample is increased from that of the one or more control sample (or to predefined reference values representing the same). Preferably the amount in the test sample is increased relative to the amount in the one or more control sample (or mean of the control samples) by at least 5%, for example, at least 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 41%, 42%, 43%, 44%, 55%, 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 125%, 150%, 175%, 200%, 225%, 250%, 275%, 300%, 350%, 400%, 500% or at least 1000% of the one or more control sample (e.g., the negative control sample).

The amount in the test sample may be increased relative to the amount in the control sample in a statistically significant manner. Any suitable means for determining p-value known to the skilled person can be used, including z-test, t-test, Student's t-test, f-test, Mann-Whitney U test, Wilcoxon signed-rank test and Pearson's chi-squared test. Preferably, the individual not afflicted with cancer was not, at the time the sample was obtained, afflicted with any disease or condition. Preferably, the individual not afflicted with cancer is a healthy individual.

Alternatively or additionally, the methods of the invention further comprise or consist of the steps of:

providing one or more control sample from;
(e) an individual afflicted with cancer (i.e., a positive control); and/or
(f) an individual afflicted with cancer, wherein the sample was of the same stage to that of that the test sample, or wherein the control sample is derived from healthy tissue from an individual afflicted with cancer;
   determining a biomarker signature of the control sample by measuring the presence and/or amount in the control sample of the all three biomarkers measured in step (b); wherein cancer is diagnosed or detected in the event that the presence and/or amount in the test sample of the biomarkers measured in step (b) corresponds to the presence and/or amount in the positive control sample of the all three biomarkers measured in step (f).

In an embodiment, alternatively or additionally, the methods of the invention further comprise or consist of the steps of:

providing one or more control sample from;
(e) an individual afflicted with cancer (i.e., a positive control); and/or
(f) an individual afflicted with cancer, wherein the sample was of the same stage to that of that the test sample;
   determining a biomarker signature of the control sample by measuring the presence and/or amount in the control sample of the all four biomarkers measured in step (b); wherein cancer is diagnosed or detected in the event that the presence and/or amount in the test sample of the biomarkers measured in step (b) corresponds to the presence and/or amount in the positive control sample of the all four biomarkers measured in step (f).

Alternatively or additionally, the sample(s) provided in step (a), (c) and/or (e) are provided before treatment of the cancer (e.g., resection, chemotherapy, radiotherapy). By “corresponds to the presence and/or amount in a positive control sample” we mean or include the presence and/or amount is identical to that of a positive control sample; or closer to that of one or more positive control sample than to one or more negative control sample (or to predefined reference values representing the same). Preferably the presence and/or amount is within ±40% of that of the one or more control sample (or mean of the control samples), for example, within ±39%, ±38%, ±37%, ±36%, ±35%, ±34%, ±33%, ±32%, ±31%, ±30%, ±29%, ±28%, ±27%, ±26%, ±25%, ±24%, ±23%, ±22%, ±21%, ±20%, ±19%, ±18%, ±17%, ±16%, ±15%, ±14%, ±13%, ±12%, ±11%, ±10%, ±9%, ±8%, ±7%, ±6%, ±5%, ±4%, ±3%, ±2%, ±1%, ±0.05% or within 0% of the one or more control sample (e.g., the positive control sample).

The difference in the presence or amount in the test sample may be ≤5 standard deviation from the mean presence or amount in the control samples, for example, ≤4.5, ≤4, ≤3.5, ≤3, ≤2.5, ≤2, ≤1.5, ≤1.4, ≤1.3, ≤1.2, ≤1.1, ≤1, ≤0.9, ≤0.8, ≤0.7, ≤0.6, ≤0.5, ≤0.4, ≤0.3, ≤0.2, ≤0.1 or 0 standard deviations from the from the mean presence or amount in the control samples, provided that the standard deviation ranges for differing and corresponding biomarker expressions do not overlap (e.g., abut, but no not overlap).

By “corresponds to the presence and/or amount in a positive control sample” we include the meaning that that the presence or amount in the test sample correlates with the amount in the control sample in a statistically significant manner. For example, the presence or amount in the test sample may correlate with that of the control sample with a p value of ≤0.05, for example, ≤0.04, ≤0.03, ≤0.02, ≤0.01, ≤0.005, ≤0.004, ≤0.003, ≤0.002, ≤0.001, ≤0.0005 or ≤0.0001.

Differential expression (up-regulation or down regulation) of biomarkers, or lack thereof, can be determined by any suitable means known to a skilled person.

Differential expression is determined to a p value of a least less than 0.05 (p=≤0.05), for example, at least ≤0.04, ≤0.03, ≤0.02, ≤0.01, ≤0.009, ≤0.005, ≤0.001, ≤0.0001, ≤0.00001 or at least ≤0.000001. Alternatively or additionally, differential expression is determined using a support vector machine (SVM).

In an embodiment, the presence and/or amount in the test sample of the one or more biomarkers measured in step (b) are compared against predetermined reference values representative of the measurements in steps (d) and/or (f).

The one or more individual afflicted with cancer may be an individual afflicted with a cancer selected from the group consisting of prostate cancer (such as castration-resistant prostate cancer), renal carcinoma, lung cancer, kidney cancer gastric cancer, bladder carcinoma, breast cancer, endometrial cancer, ovarian cancer, and colorectal cancer. Preferably, the individual afflicted with cancer is one who is known to have the same type of cancer as the cancer that is to be diagnosed or detected. The one or more individual afflicted with cancer may be afflicted with a cancer associated with and/or mediated by proteolytic cleavage of transforming growth factor β type I receptor (TβRI).

In an embodiment, in the event that the subject is diagnosed with cancer, the method further comprises the step of:
providing the subject with cancer therapy.

Preferably, the cancer therapy is selected from the group consisting of surgery, chemotherapy, immunotherapy, chemoimmunotherapy and thermochemotherapy. Accordingly, in one embodiment, where the presence of cancer is indicated, the method comprises treating the subject for cancer according to current recommendations (e.g., surgical removal of cancer cells, radiotherapy and/or chemotherapy).

In an embodiment, the cancer therapy is selected from the group consisting of surgery, chemotherapy, immunotherapy, chemoimmunotherapy and thermochemotherapy (e.g., AC chemotherapy; Capecitabine and docetaxel chemotherapy (Taxotere®); CMF chemotherapy; Cyclophosphamide; EC chemotherapy; ECF chemotherapy; E-CMF chemotherapy (Epi-CMF); Eribulin (Halaven®); FEC chemotherapy; FEC-T chemotherapy; Fluorouracil (5FU); GemCarbo chemotherapy; Gemcitabine (Gemzar®); Gemcitabine and cisplatin chemotherapy (GemCis or GemCisplat); GemTaxol chemotherapy; Idarubicin (Zavedos®); Liposomal doxorubicin (DaunoXome®); Mitomycin (Mitomycin C Kyowa®); Mitoxantrone; MM chemotherapy; MMM chemotherapy; Paclitaxel (Taxol®); TAC chemotherapy; Taxotere and cyclophosphamide (TC) chemotherapy; Vinblastine (Velbe®); Vincristine (Oncovin®); Vindesine (Eldisine®); and Vinorelbine (Navelbine®)).

In an embodiment, the anti-cancer agent is an agent that prevents cleavage of TβRI, preferably so that the intracellular domain is incapable of translocating to the nucleus, such as an antibody or antigen-binding fragment thereof, or a small molecule that prevents cleavage of TβRI.

Alternatively or additionally, the method is repeated.

Alternatively or additionally, the method is repeated wherein, in step (a), the sample to be tested is one that has been obtained from the subject at a different time to the sample in the previous method repetition.

It will be understood that the method is repeated using a test sample taken at a different time period to the previous test sample(s) used.

Alternatively or additionally, the method is repeated using a test sample taken between 1 day to 104 weeks to the previous test sample(s) used, for example, between 1 week to 100 weeks, 1 week to 90 weeks, 1 week to 80 weeks, 1 week to 70 weeks, 1 week to 60 weeks, 1 week to 50 weeks, 1 week to 40 weeks, 1 week to 30 weeks, 1 week to 20 weeks, 1 week to 10 weeks, 1 week to 9 weeks, 1 week to 8 weeks, 1 week to 7 weeks, 1 week to 6 weeks, 1 week to 5 weeks, 1 week to 4 weeks, 1 week to 3 weeks, or 1 week to 2 weeks.

Alternatively or additionally, the method is repeated using a test sample taken every period from the group consisting of: 1 day, 2 days, 3 day, 4 days, 5 days, 6 days, 7 days, 10 days, 2 weeks, 3 weeks, 4 weeks, 5 weeks, 6 weeks, 7 weeks, 8 weeks, 9 weeks, 10 weeks, 15 weeks, 20 weeks, 25 weeks, 30 weeks, 35 weeks, 40 weeks, 45 weeks, 50 weeks, 55 weeks, 60 weeks, 65 weeks, 70 weeks, 75 weeks, 80 weeks, 85 weeks, 90 weeks, 95 weeks, 100 weeks, 104, weeks, 105 weeks, 110 weeks, 115 weeks, 120 weeks, 125 weeks and 130 weeks.

Alternatively or additionally, the method is repeated at least once, for example, 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, 21 times, 22 times, 23, 24 times or 25 times.

In a further object, the invention provides a method for determining the Gleason score (GS) in a subject suffering from, or suspected to be suffering from prostate cancer, as being either (i) GS≤6 or 7 (3+4); or (ii) GS 7 (4+3) or ≥8, the method comprising the steps of:
a) providing a biological test sample from the subject;
b) assessing the amount of a complex comprising Aurora kinase B (AURKB) and TGFβ receptor type 1 (TβR1);
c) comparing the amount of the complex in (b) with the amount of a complex comprising Aurora kinase B (AURKB) and TGFβ receptor type 1 (TβR1) from a reference sample that is known to have a GS of either (i) GS≤6 or 7 (3+4); or (ii) GS 7 (4+3) or ≥8;
   wherein the comparison allows the determination of the GS in the subject as being either (i) GS≤6 or 7 (3+4); or (ii) GS 7 (4+3) or ≥8.

By a “complex comprising Aurora kinase B (AURKB) and TGFβ receptor type 1 (TβR1)”, we include the meaning of a collection of two or more proteins that interact with each other to form a multiprotein structure at the same location, which two or more proteins comprise Aurora kinase B (AURKB) and TGFβ receptor type 1 (TβR1). Preferably, the proteins in the complex interact with each other by means of non-covalent interactions. Methods of detecting protein complexes are well known in the art and include but are not limited to, immunoprecipitation and in situ proximity ligation assays (PLA), immunofluorescence and confocal microscopy. Such protein complexes can then be quantified using methods known in the art and those described in the accompanying Examples.

In an embodiment of any of the methods described herein, the TGFβ receptor type 1 (TβR1) is the intracellular domain (TβR1-ICD).

Currently, the most common grading system for prostate cancer is the Gleason grading system, which is used to indicate how likely it is that a tumor will spread based on its microscopic appearance (Gleason and Mellinger, 1974, Iczkowski K A. Gleason grading. PathologyOutlines.com website. https://www.pathologyoutlines.com/topic/prostategrading.html.). The tissue can be stained with antibodies against α-methylacyl-CoA racemase (AMACR), p63 and cytokeratin (CK) 5 and investigated using light microscopy. This system uses a scale from 1 to 5, where 5 represents the more aggressive tumor pattern. Two grades are given, one to the most common area and the other to the second most common area, respectively. Then the pathologist adds together the two grades to obtain the “Gleason Score” (GS). The GS ranges from 2 to 10 and has a very strong prognostic value as a predictor of death from prostate cancer. Patients with a high GS (8-10) have worse survival outcomes.

Because of the different proportion of Gleason pattern 3 and Gleason pattern 4 leading to various prognosis, in 2014, a new grading system was proposed that separated a GS of 7 into two different groups: GS 3+4=7 (prognostic grade group II) and GS 4+3=7(prognostic grade group III), (Pierorazio P M, et al. BJU Int. (2013) 111:753-60). Being able to distinguish between GS 3+4=7 and GS 4+3=7, is important as there are different radiation therapy approaches for GS 3+4=7 (Grade Group II) versus 4+3=7 (Grade Group III) (Zhu et al. Front. Oncol., 16 July 2019).

In an embodiment, the method is capable of distinguishing a test sample from a subject having a Gleason score of ≤6 or 7 (3+4) from a test sample from a subject having a Gleason score of 7 (4+3) or ≥8. As shown in the accompanying Examples, the inventors surprisingly found that a high number of AURKB-TβRI-ICD complexes were found in clinical material of prostate cancer patients with high Gleason Score (7=(4+3) or ≥8) compared to clinical material of prostate cancer patients with lower Gleason Score (7=(3+4) or ≤6) (FIG. 5B).

In an embodiment, the complex further comprises Adaptor Protein, Phosphotyrosine Interacting With PH Domain And Leucine Zipper 1 (APPL1).

In an embodiment, the complex further comprises TNF receptor associated factor 6 (TRAF6).

In an embodiment, the complex is localised to a cellular structure, such as a cytokinesis structure.

Preferably, the AURKB is ubiquitinated.

In an embodiment, Aurora kinase B (AURKB) is ubiquitinated at one or both lysine residues corresponding to Lysine 85 (K85) and/or Lysine 87 (K87) of human AURKB (SEQ ID NO: 1).

In an embodiment, the prostate cancer is castration-resistant prostate cancer (CRPC). In a further aspect, the invention provides an array for determining the presence of cancer in a subject comprising:
(i) a binding agent capable of binding to Aurora kinase B (AURKB) as described herein and/or a binding moiety capable of binding selectively to a nucleic acid molecule encoding capable of binding to Aurora kinase B (AURKB) as described herein;
(ii) a binding agent capable of binding to Adaptor Protein, Phosphotyrosine Interacting With PH Domain And Leucine Zipper 1 (APPL1) as described herein and/or a binding moiety capable of binding selectively to a nucleic acid molecule encoding capable of binding to Adaptor Protein, Phosphotyrosine Interacting With PH Domain And Leucine Zipper 1 (APPL1) as described herein; and
(iii) a binding agent capable of binding to TGFβ receptor type 1 (TβR1) as described herein and/or a binding moiety capable of binding selectively to a nucleic acid molecule encoding capable of binding to TGFβ receptor type 1 (TβR1) as described herein.

In an embodiment, the array further comprises (iv) a binding agent capable of binding to TNF receptor associated factor 6 (TRAF6) as described herein and/or a binding moiety capable of binding selectively to a nucleic acid molecule encoding capable of binding to TNF receptor associated factor 6 (TRAF6) as described herein.

In a further aspect, the invention provides an array for determining the presence of cancer in a subject comprising:
(i) a binding agent capable of binding to Aurora kinase B (AURKB) as described herein and/or a binding moiety capable of binding selectively to a nucleic acid molecule encoding capable of binding to Aurora kinase B (AURKB) as described herein;
(ii) a binding agent capable of binding to Adaptor Protein, Phosphotyrosine Interacting With PH Domain And Leucine Zipper 1 (APPL1) as described herein and/or a binding moiety capable of binding selectively to a nucleic acid molecule encoding capable of binding to Adaptor Protein, Phosphotyrosine Interacting With PH Domain And Leucine Zipper 1 (APPL1) as described herein;
(iii) a binding agent capable of binding to TGFβ receptor type 1 (TβR1) as described herein and/or a binding moiety capable of binding selectively to a nucleic acid molecule encoding capable of binding to TGFβ receptor type 1 (TβR1) as described herein; and
(iv) a binding agent capable of binding to TNF receptor associated factor 6 (TRAF6) as described herein and/or a binding moiety capable of binding selectively to a nucleic acid molecule encoding capable of binding to TNF receptor associated factor 6 (TRAF6) as described herein.

The cancer may be an aggressive cancer.

Preferably, the binding agent is capable of binding to the TGFβ receptor type 1 intracellular domain (TβR1-ICD).

In an embodiment, the binding agent in (i) is capable of binding to ubiquitinated Aurora kinase B (AURKB) as described herein. In an embodiment, the binding agent in (i) is capable of distinguishing ubiquitinated Aurora kinase B (AURKB) from AURKB that is not ubiquitinated.

Once suitable binding molecules (discussed above) have been identified and isolated, the skilled person can manufacture an array using methods well known in the art of molecular biology. An array is typically formed of a linear or two-dimensional structure having spaced apart (i.e. discrete) regions (“spots”), each having a finite area, formed on the surface of a solid support. An array can also be a bead structure where each bead can be identified by a molecular code or colour code or identified in a continuous flow. Analysis can also be performed sequentially where the sample is passed over a series of spots each adsorbing the class of molecules from the solution. The solid support is typically glass or a polymer, the most commonly used polymers being cellulose, polyacrylamide, nylon, polystyrene, polyvinyl chloride or polypropylene. The solid supports may be in the form of tubes, beads, discs, silicon chips, microplates, polyvinylidene difluoride (PVDF) membrane, nitrocellulose membrane, nylon membrane, other porous membrane, non-porous membrane (e.g. plastic, polymer, perspex, silicon, amongst others), a plurality of polymeric pins, or a plurality of microtitre wells, or any other surface suitable for immobilizing proteins, polynucleotides and other suitable molecules and/or conducting an immunoassay. The binding processes are well known in the art and generally consist of cross-linking covalently binding or physically adsorbing a protein molecule, polynucleotide or the like to the solid support. By using well-known techniques, such as contact or non-contact printing, masking or photolithography, the location of each spot can be defined. For reviews see Jenkins, R. E., Pennington, S. R. (2001, Proteomics, 2,13-29) and Lal et al (2002, Drug Discov Today 15; 7(18 Suppl): S143-9).

Typically, the array is a microarray. By “microarray” we include the meaning of an array of regions having a density of discrete regions of at least about 100/cm², and preferably at least about 1000/cm². The regions in a microarray have typical dimensions, e.g., diameters, in the range of between about 10-250 μm, and are separated from other regions in the array by about the same distance. The array may also be a macroarray or a nanoarray.

Once suitable binding molecules (discussed above) have been identified and isolated, the skilled person can manufacture an array using methods well known in the art of molecular biology.

In an embodiment, the array comprises one or more antibodies, or antigen-binding fragments thereof, capable (individually or collectively) of binding said biomarkers Aurora kinase B (AURKB), Adaptor Protein, Phosphotyrosine Interacting With PH Domain And Leucine Zipper 1 (APPL1), TGFβ receptor type 1 (TβR1) or its intracellular domain (TβR1-ICD) and TNF receptor associated factor 6 (TRAF6) at the protein level. For example, the array may comprise scFv molecules capable (collectively) of binding to all biomarkers Aurora kinase B (AURKB), Adaptor Protein, Phosphotyrosine Interacting With PH Domain And Leucine Zipper 1 (APPL1), TGFβ receptor type 1 (TβR1) or its intracellular domain (TβR1-ICD) and TNF receptor associated factor 6 (TRAF6) at the protein level.

It will be appreciated that the array may comprise one or more positive and/or negative control samples, such as the control samples described herein.

It is a further object to provide a kit for the diagnosis and/or prognosis of in a subject said kit comprising:
(i) a binding agent capable of binding to Aurora kinase B (AURKB) as described herein and/or a binding moiety capable of binding selectively to a nucleic acid molecule encoding Aurora kinase B (AURKB) as described herein;
(ii) a binding agent capable of binding to Adaptor Protein, Phosphotyrosine Interacting With PH Domain And Leucine Zipper 1 (APPL1) as described herein and/or a binding moiety capable of binding selectively to a nucleic acid molecule encoding Adaptor Protein, Phosphotyrosine Interacting With PH Domain And Leucine Zipper 1 (APPL1) as described herein; and
(iii) a binding agent capable of binding to TGFβ receptor type 1 (TβR1) or its intracellular domain (ICD) as described herein and/or a binding moiety capable of binding selectively to a nucleic acid molecule encoding TGFβ receptor type 1 (TβR1) or its intracellular domain (ICD) as described herein.

In an embodiment, the kit further comprises (iv) a binding agent capable of binding to TNF receptor associated factor 6 (TRAF6) as described herein and/or a binding moiety capable of binding selectively to a nucleic acid molecule encoding capable of binding to TNF receptor associated factor 6 (TRAF6) as described herein.

It is a further object to provide a kit for the diagnosis and/or prognosis of in a subject said kit comprising:
(i) a binding agent capable of binding to Aurora kinase B (AURKB) and/or a binding moiety capable of binding selectively to a nucleic acid molecule encoding Aurora kinase B (AURKB) as described herein;
(ii) a binding agent capable of binding to Adaptor Protein, Phosphotyrosine Interacting With PH Domain And Leucine Zipper 1 (APPL1) and/or a binding moiety capable of binding selectively to a nucleic acid molecule encoding Adaptor Protein, Phosphotyrosine Interacting With PH Domain And Leucine Zipper 1 (APPL1) as described herein;
(iii) a binding agent capable of binding to TGFβ receptor type 1 (TβR1) and/or a binding moiety capable of binding selectively to a nucleic acid molecule encoding TGFβ receptor type 1 (TβR1) as described herein; and
(iv) a binding agent capable of binding to TNF receptor associated factor 6 (TRAF6) and/or a binding moiety capable of binding selectively to a nucleic acid molecule encoding TNF receptor associated factor 6 (TRAF6) as described herein.

Optionally, the kit further comprises instruction for use.

The kit for example is suitable for the diagnosis and/or prognosis of cancer. The cancer may be a solid tumour. The tumour may for example be selected from the group consisting of prostate cancer, renal carcinoma, lung cancer, gastric cancer, bladder carcinoma, breast cancer, endometrial cancer, ovarian cancer and colorectal cancer. The cancer may be an aggressive cancer.

In an embodiment the TGFβ receptor type 1 (TβR1) is the intracellular domain (TβR1-ICD).

In an embodiment, the binding agent capable in (i) is capable of binding to ubiquitinated Aurora kinase B (AURKB) as described herein. In an embodiment, the binding agent in (i) is capable of distinguishing ubiquitinated Aurora kinase B (AURKB) from AURKB that is not ubiquitinated.

As with the array, it will be appreciated that the kit may comprise one or more positive and/or negative control samples, for example as described herein.

It is a further object to provide, Aurora kinase B (AURKB), Adaptor Protein, Phosphotyrosine Interacting With PH Domain And Leucine Zipper 1 (APPL1), and TGFβ receptor type 1 (TβR1) for use as biomarkers in the diagnosis and/or prognosis of a disease or condition involving proteolytic cleavage of TGFβ receptor type 1, wherein the co-localization of all three markers to a cytokinesis structure in a cell is indicative of said disease or condition.

In an embodiment, Aurora kinase B (AURKB), Adaptor Protein, Phosphotyrosine Interacting With PH Domain And Leucine Zipper 1 (APPL1), and TGFβ receptor type 1 (TβR1) for use as biomarkers, further comprises TNF receptor associated factor 6 (TRAF6) for use as a biomarker in the diagnosis and/or prognosis of a disease or condition involving proteolytic cleavage of TGFβ receptor type 1, wherein the co-localization of all four biomarkers to a cytokinesis structure in a cell is indicative of said disease or condition.

It is a further object to provide, Aurora kinase B (AURKB), Adaptor Protein, Phosphotyrosine Interacting With PH Domain And Leucine Zipper 1 (APPL1), TGFβ receptor type 1 (TβR1) and TNF receptor associated factor 6 (TRAF6) for use as biomarkers in the diagnosis a disease or condition involving proteolytic cleavage of TGFβ receptor type 1, wherein the co-localization of all four markers to a cytokinesis structure in a cell is indicative of said disease or condition.

In an embodiment, the disease or condition involving proteolytic cleavage of TGFβ receptor type 1 is cancer. In an embodiment, the cancer is any of the cancers described herein. Preferably, AURKB is ubiquitinated. In an embodiment the TGFβ receptor type 1 (TβR1) is the intracellular domain (TβR1-ICD).

It is a further object to provide, use of Aurora kinase B (AURKB), Adaptor Protein, Phosphotyrosine Interacting With PH Domain And Leucine Zipper 1 (APPL1), and TGFβ receptor type 1 (TβR1) as biomarkers for the diagnosis and/or prognosis of a disease or condition involving proteolytic cleavage of TGFβ receptor type 1.

In an embodiment, the use further comprises the use of TNF receptor associated factor 6 (TRAF6) as a biomarker for the diagnosis and/or prognosis of a disease or condition involving proteolytic cleavage of TGFβ receptor type 1.

It is a further object to provide, use of Aurora kinase B (AURKB), Adaptor Protein, Phosphotyrosine Interacting With PH Domain And Leucine Zipper 1 (APPL1), TGFβ receptor type 1 (TβR1) and TNF receptor associated factor 6 (TRAF6) as biomarkers for determining the presence of cancer in a subject.

In an embodiment, the use comprises providing a biological test sample from a subject to be tested, and optionally a control sample, as described herein.

It is a further object to provide, a complex comprising Aurora kinase B (AURKB), Adaptor Protein, Phosphotyrosine Interacting With PH Domain And Leucine Zipper 1 (APPL1), and TGFβ receptor type 1 (TβR1), wherein AURKB is ubiquitinated. In an embodiment, the complex further comprises TNF receptor associated factor 6 (TRAF6).

It is a further object to provide, a complex comprising Aurora kinase B (AURKB), Adaptor Protein, Phosphotyrosine Interacting With PH Domain And Leucine Zipper 1 (APPL1), TGFβ receptor type 1 (TβR1) and TNF receptor associated factor 6 (TRAF6), wherein AURKB is ubiquitinated.

In an embodiment the TGFβ receptor type 1 (TβR1) is the intracellular domain (TβR1-ICD). Preferably, AURKB is ubiquitinated.

In an embodiment, wherein in the event that the subject is diagnosed with cancer and/or aggressive cancer, the method further comprises the step of:
administering a cancer therapy to the subject.

It is a further object to provide, a method for treating cancer in a subject, which subject has been diagnosed as having a cancer according to the methods described herein, the method comprising administering a cancer therapy to the subject. Suitable cancer therapies are known in the art and are discussed herein. In an embodiment, the anti-cancer agent is an antibody or antigen-binding fragment thereof or a small molecule that prevents cleavage of TβRI.

Preferably, the method comprises the following steps:
(a) diagnosing a subject as having a cancer using a method of the invention; and
(b) treating the subject so diagnosed with a cancer therapy.

In an embodiment, the anticancer agent is administered in combination with another cancer therapy, either simultaneously or sequentially.

In an embodiment, the subject may be administered an effective amount of a cancer therapy and/or anticancer agent. By “effective amount” we include the meaning of an amount of a pharmaceutical compound or composition which is effective to achieve an improvement in a subject, including but not limited to, improved survival rate, more rapid recovery, or improvement or elimination of symptoms, and/or other indicators as selected by those skilled in the art.

Further, there is provided a method for monitoring a treatment of a subject having cancer. The method is suitable for cancers mediated by proteolytic cleavage of transforming growth factor β type I receptor (TβRI). The method comprises the steps of:
providing a first biological sample s₁ from the subject to be tested;
determining a first value v₁ representing the expression level of a first biomarker, a second biomarker, and a third biomarker, wherein said biomarkers are: Aurora kinase B (AURKB), Adaptor Protein, Phosphotyrosine Interacting With PH Domain And Leucine Zipper 1 (APPL1), and TGFβ receptor type 1 (TβR1) in the first biological sample at a first time point t₁ of a treatment;
starting or continuing the treatment;
obtaining a second biological sample s₂ from said subject after a predetermined time t₂ of treatment, and
determining a second value v₂ representing the expression level of a first biomarker, a second biomarker, and a third biomarker, wherein said biomarkers are: Aurora kinase B (AURKB), Adaptor Protein, Phosphotyrosine Interacting With PH Domain And Leucine Zipper 1 (APPL1), and TGFβ receptor type 1 (TβR1) in the second biological sample at time t₂ of treatment, and
if the level of v₁>v₂ the subject is responding to the treatment, if v₁<v₂ the subject is not responding to the treatment.

Further, there is provided a method for monitoring a treatment of a subject having cancer. The method is suitable for cancers mediated by proteolytic cleavage of transforming growth factor β type I receptor (TβRI). The method comprises the steps of:
providing a first biological sample s₁ from the subject to be tested;
determining a first value v₁ representing the expression level of a first biomarker, a second biomarker, a third biomarker, and a fourth biomarker, the biomarkers are: Aurora kinase B (AURKB), Adaptor Protein, Phosphotyrosine Interacting With PH Domain And Leucine Zipper 1 (APPL1), TGFβ receptor type 1 (TβR1) and TNF receptor associated factor 6 (TRAF6) in the first biological sample at a first time point t₁ of a treatment;
starting or continuing the treatment;
obtaining a second biological sample s₂ from said subject after a predetermined time t₂ of treatment, and
determining a second value v₂ representing the expression level of a first biomarker, a second biomarker, a third biomarker, and a fourth biomarker, the biomarkers are: Aurora kinase B (AURKB), Adaptor Protein, Phosphotyrosine Interacting With PH Domain And Leucine Zipper 1 (APPL1), TGFβ receptor type 1 (TβR1) and TNF receptor associated factor 6 (TRAF6) in the second biological sample at time t₂ of treatment, and
if the level of v₁>v₂ the subject is responding to the treatment, if v₁<v₂ the subject is not responding to the treatment.

A decreased expression level of said biomarkers compared to a reference value is indicative of a reduced number of cancer cells.

Further, there is provided a method for monitoring a treatment of a subject having cancer. The method is suitable for cancers mediated by proteolytic cleavage of transforming growth factor β type I receptor (TβRI). The method comprises the steps of:
providing a first biological sample s₁ from the subject to be tested;
determining a first value v₁ representing the co-localization of a first biomarker, a second biomarker, and a third biomarker, wherein said biomarkers are: Aurora kinase B (AURKB), Adaptor Protein, Phosphotyrosine Interacting With PH Domain And Leucine Zipper 1 (APPL1), and TGFβ receptor type 1 (TβR1) in the first biological sample at a first time point t₁ of a treatment;
starting or continuing the treatment;
obtaining a second biological sample s₂ from said subject after a predetermined time t₂ of treatment, and
determining a second value v₂ representing the co-localization of a first biomarker, a second biomarker, and a third biomarker, wherein said biomarkers are: Aurora kinase B (AURKB), Adaptor Protein, Phosphotyrosine Interacting With PH Domain And Leucine Zipper 1 (APPL1), and TGFβ receptor type 1 (TβR1) in the second biological sample at time t₂ of treatment, and
if the level of v₁>v₂ the subject is responding to the treatment, if v₁<v₂ the subject is not responding to the treatment.

Further, there is provided a method for monitoring a treatment of a subject having cancer. The method is suitable for cancers mediated by proteolytic cleavage of transforming growth factor β type I receptor (TβRI). The method comprises the steps of:
providing a first biological sample s₁ from the subject to be tested;
determining a first value v₁ representing the co-localization of a of a first biomarker, a second biomarker, a third biomarker, and a fourth biomarker, the biomarkers are: Aurora kinase B (AURKB), Adaptor Protein, Phosphotyrosine Interacting With PH Domain And Leucine Zipper 1 (APPL1), TGFβ receptor type 1 (TβR1) and TNF receptor associated factor 6 (TRAF6) in the first biological sample at a first time point t₁ of a treatment;
starting or continuing the treatment;
obtaining a second biological sample s₂ from said subject after a predetermined time t₂ of treatment, and
determining a second value v₂ representing the co-localization of a of a first biomarker, a second biomarker, a third biomarker, and a fourth biomarker, the biomarkers are: Aurora kinase B (AURKB), Adaptor Protein, Phosphotyrosine Interacting With PH Domain And Leucine Zipper 1 (APPL1), TGFβ receptor type 1 (TβR1) and TNF receptor associated factor 6 (TRAF6) in the second biological sample at time t₂ of treatment, and
if the level of v₁>v₂ the subject is responding to the treatment, if v₁<v₂ the subject is not responding to the treatment.

A decreased level of colocalization of said biomarkers compared to a reference value is indicative of a reduced number of cancer cells.

The reference value may be for example before start of a treatment, after a change of a treatment or any change that may be of interest to monitor, i.e., start value t₀.

The second, third, fourth, fifth etc. value may be set at a predetermined time point after the start point t₀ or change of a treatment, at predetermined time points during a treatment or other interesting events that are to be monitored.

The method(s) and kit(s) described above are not limited to be used only in view of cancer diseases, the methods and kits may be useful for any other disease or condition associated with and/or mediated by the proteolytic cleavage of transforming growth factor β type I receptor (TβRI).

The present invention will now be described by reference to the following Figures and Examples.

### BRIEF DESCRIPTION OF THE DRAWINGS

FIG. 1 APPL1 and 2 promote AURKB, BIRC5, CDCA8, and KIF2C expression.

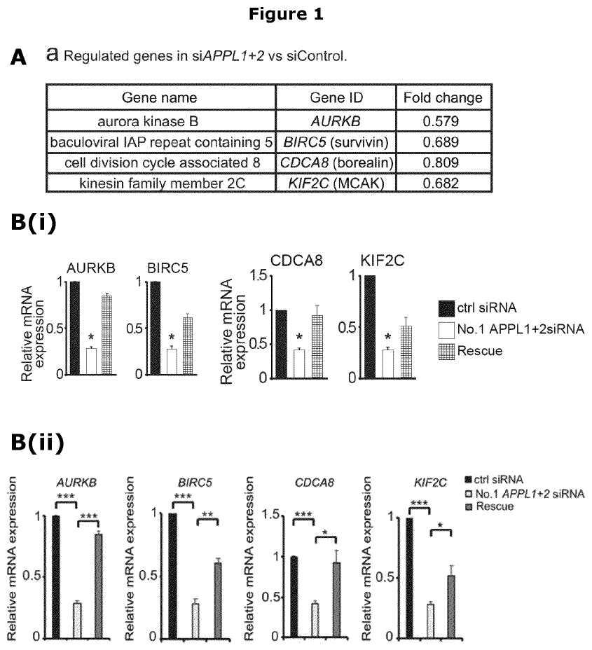

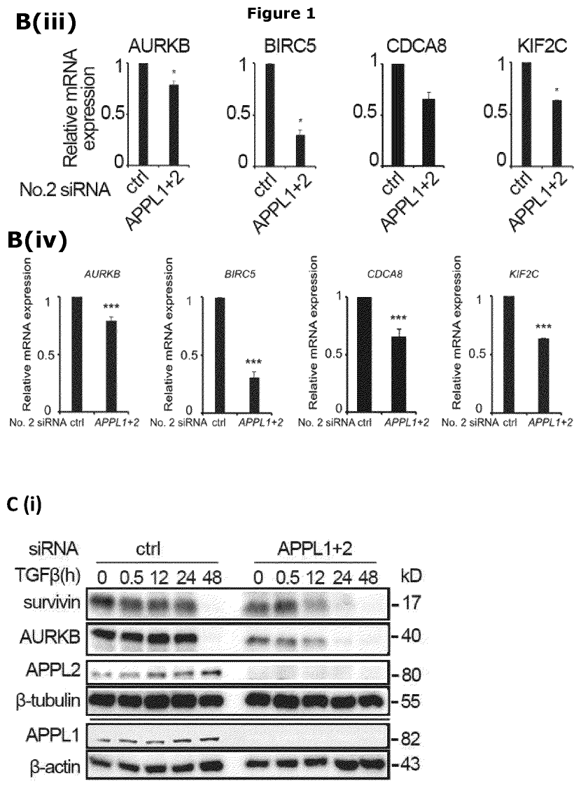

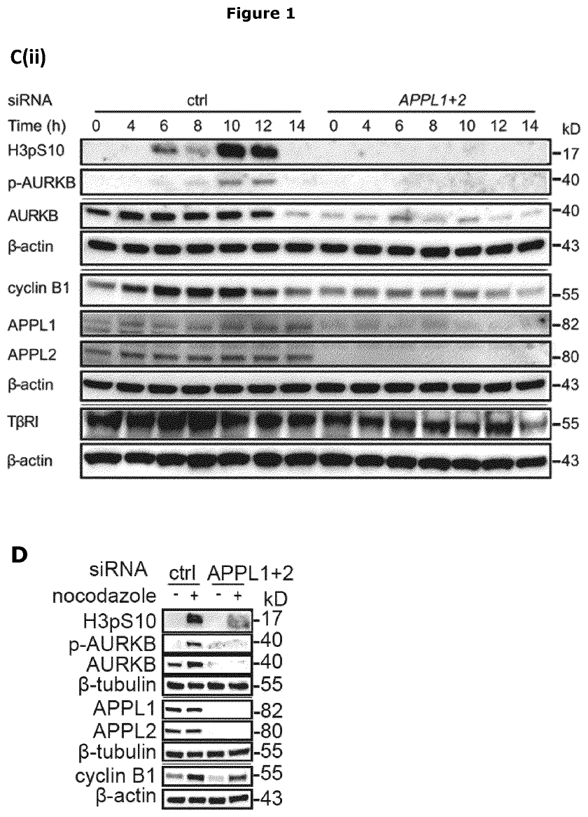

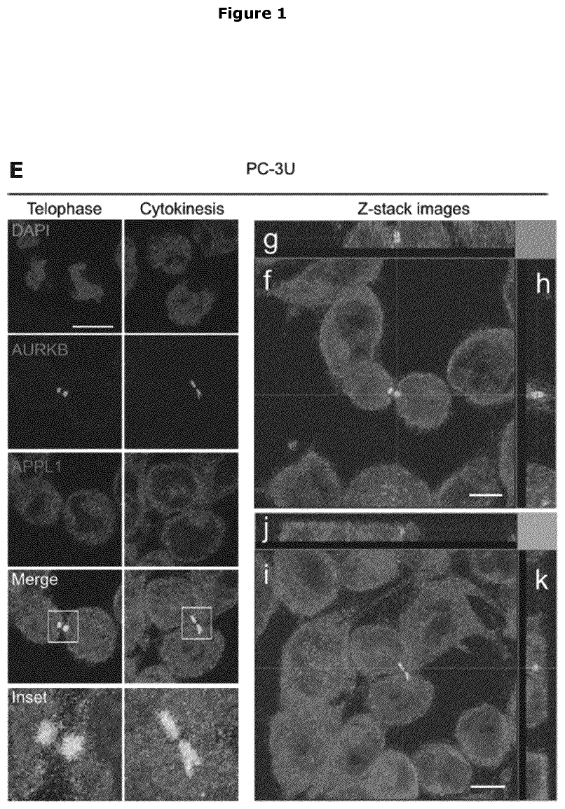

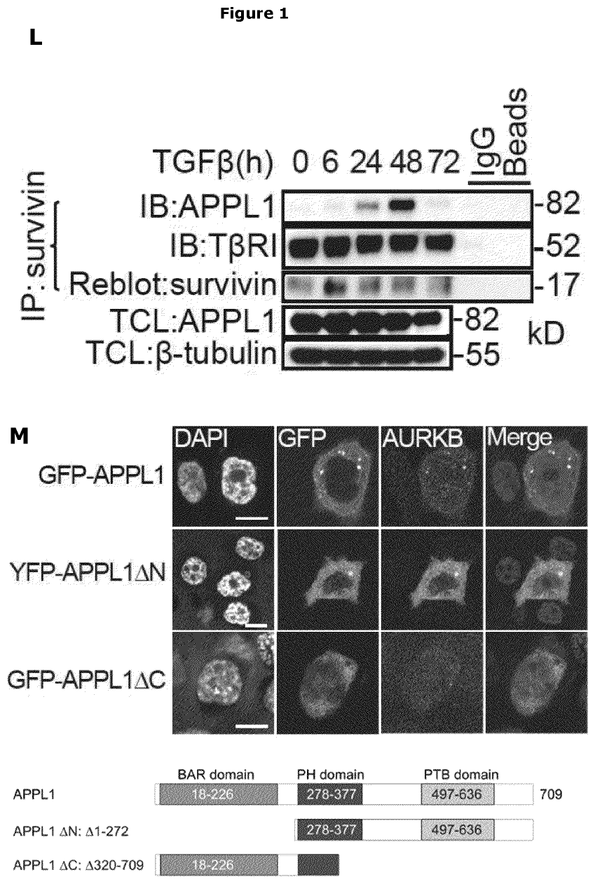

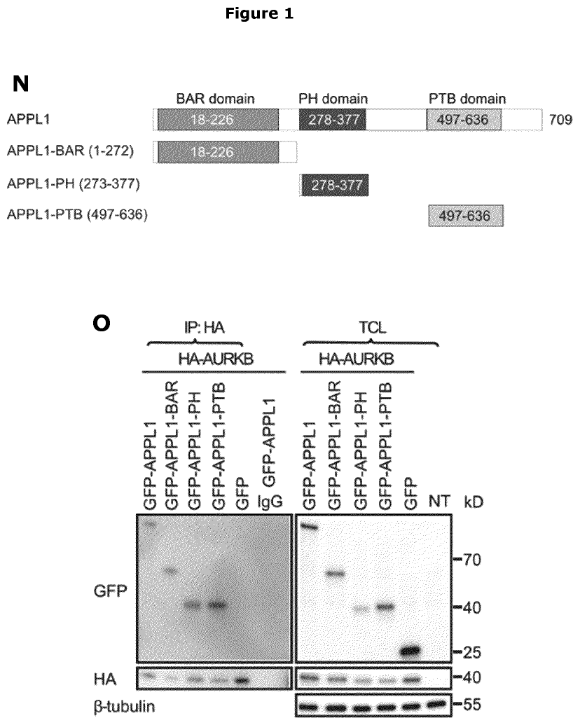

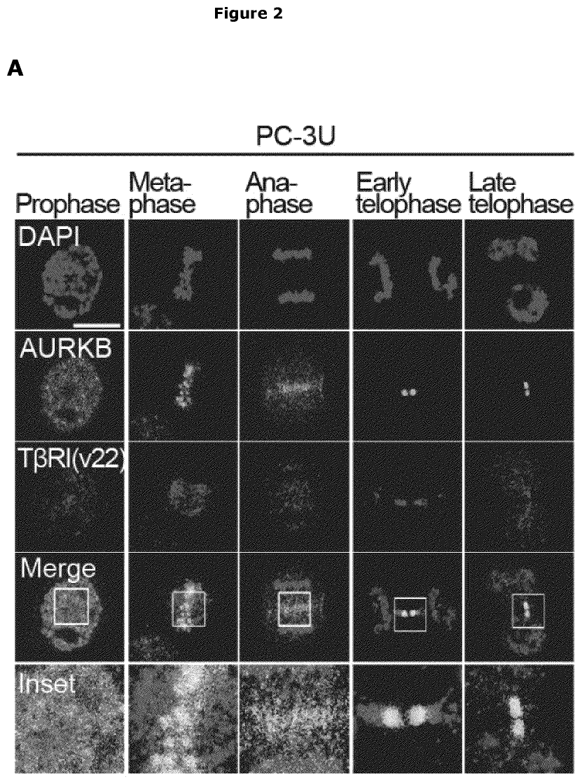

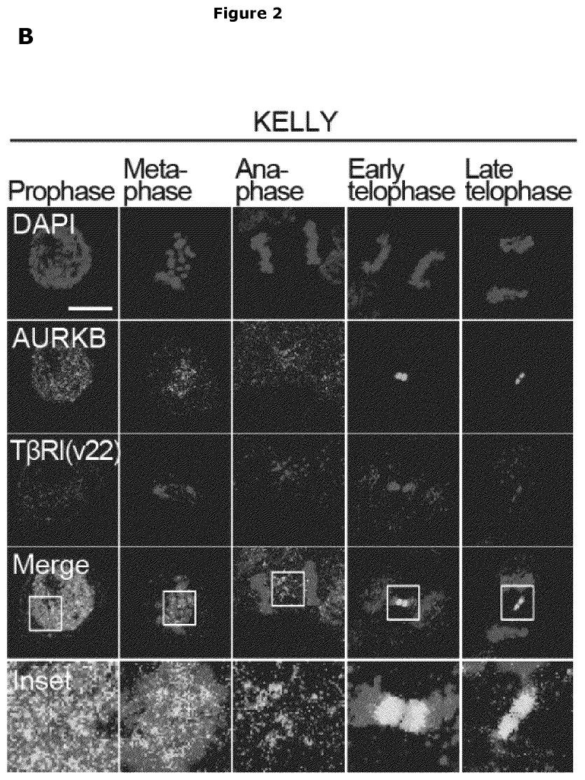

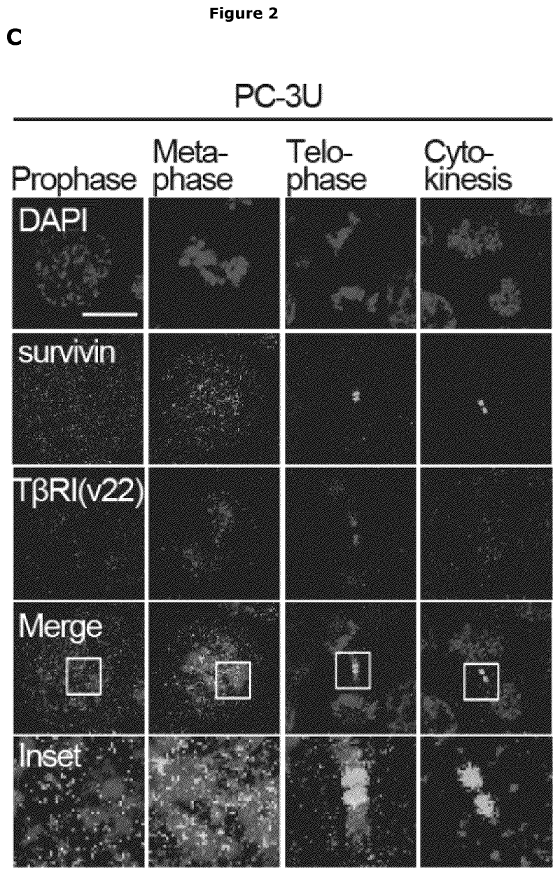

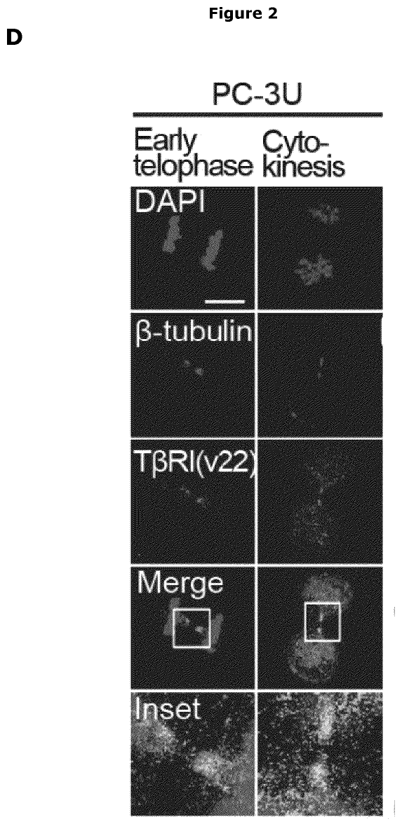

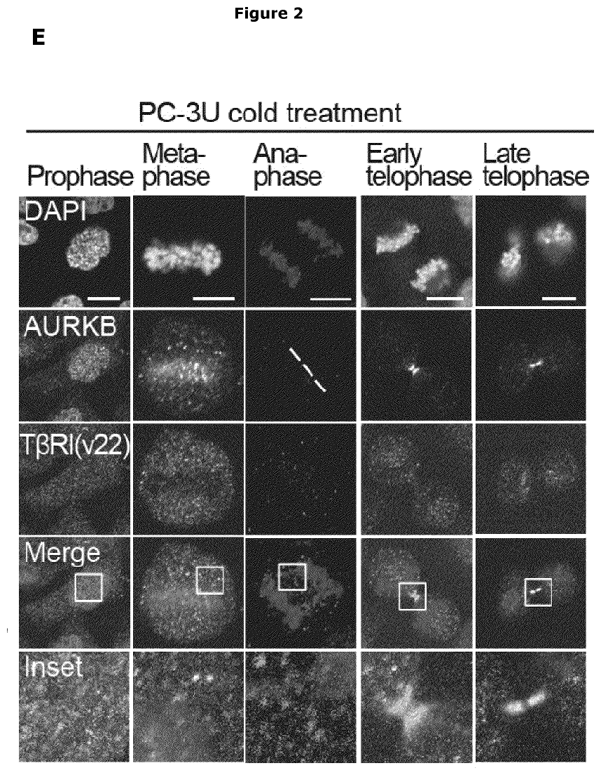

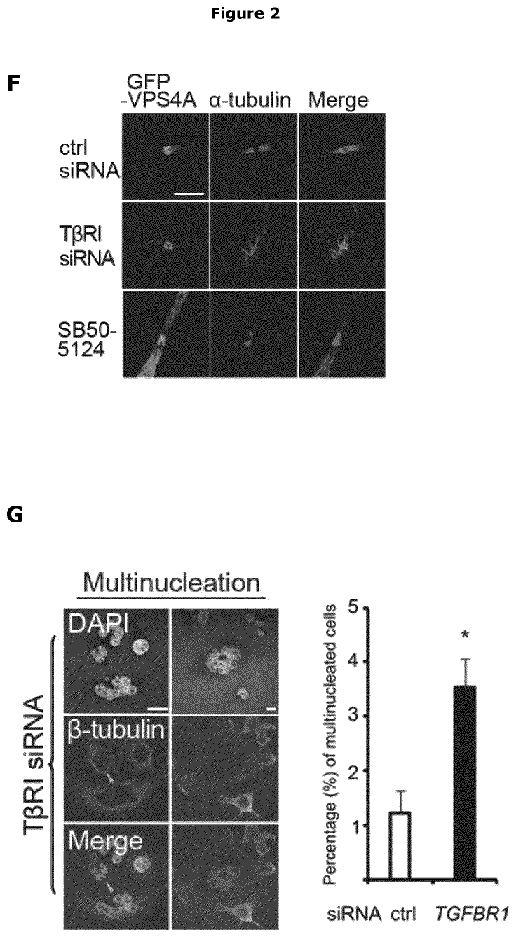

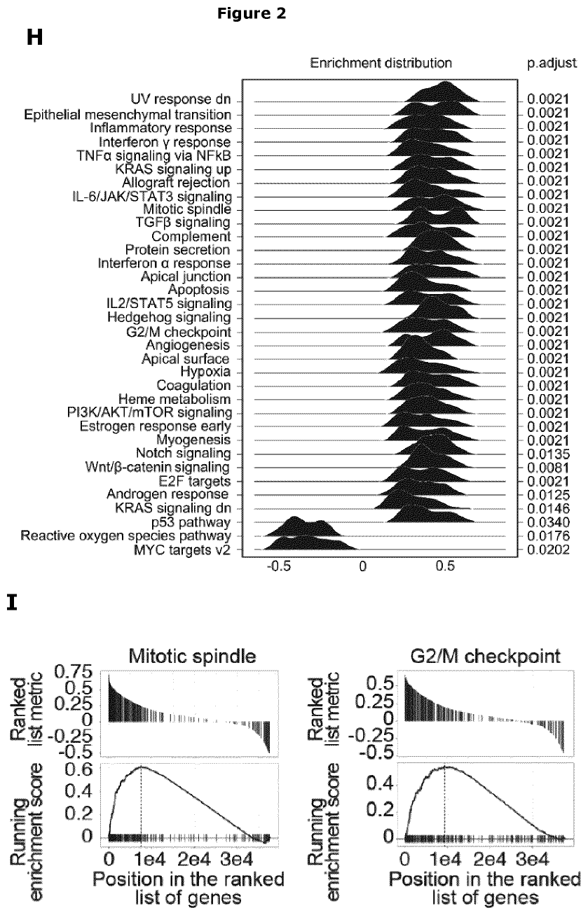

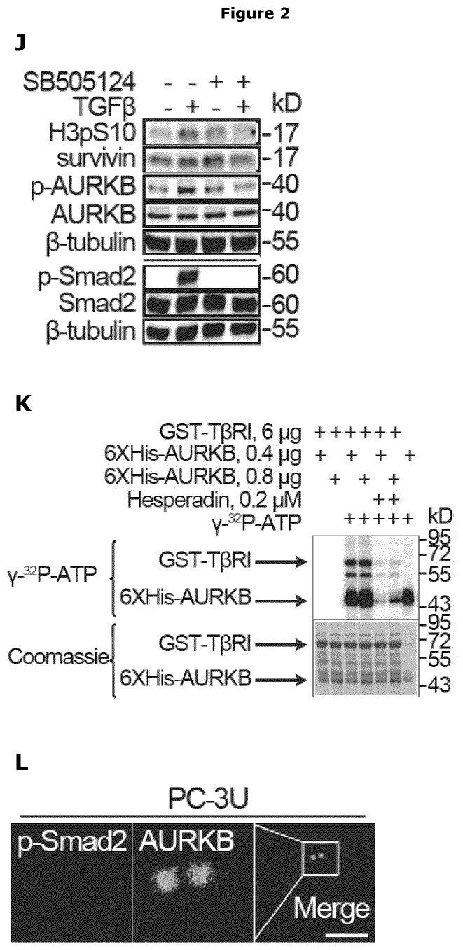

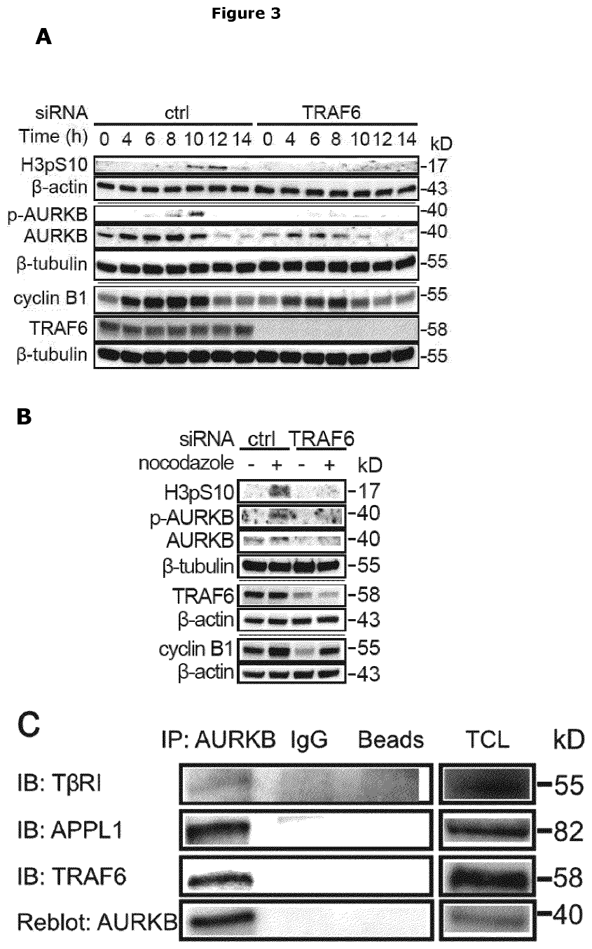

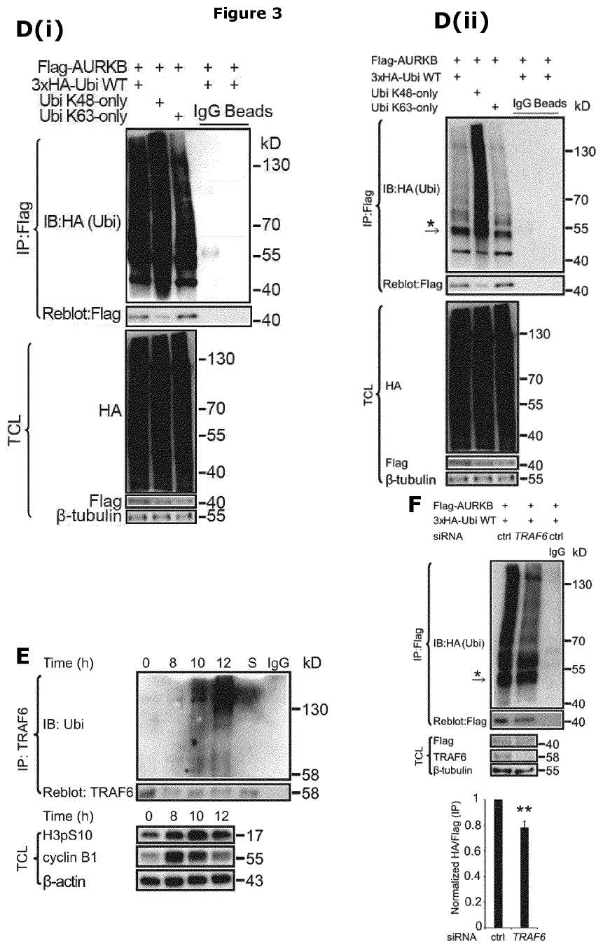

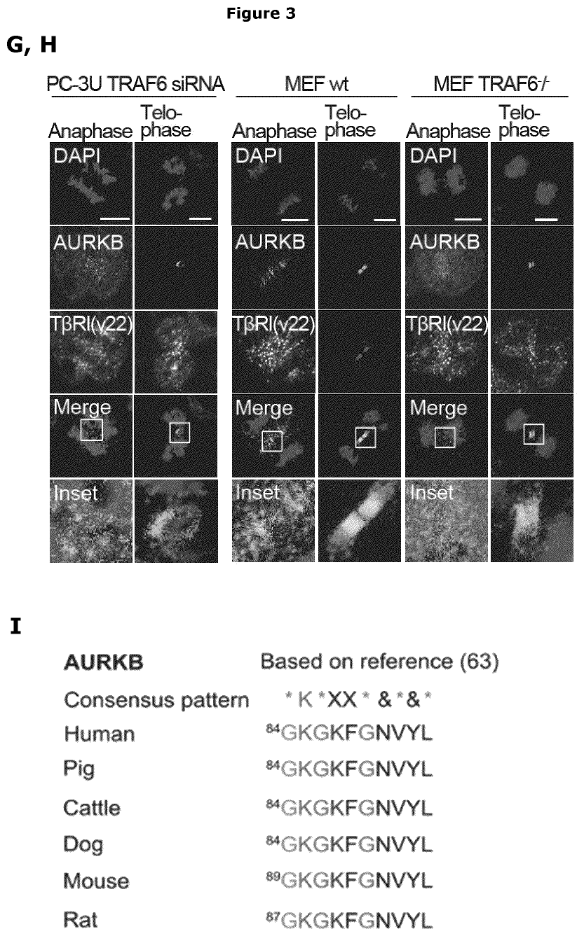

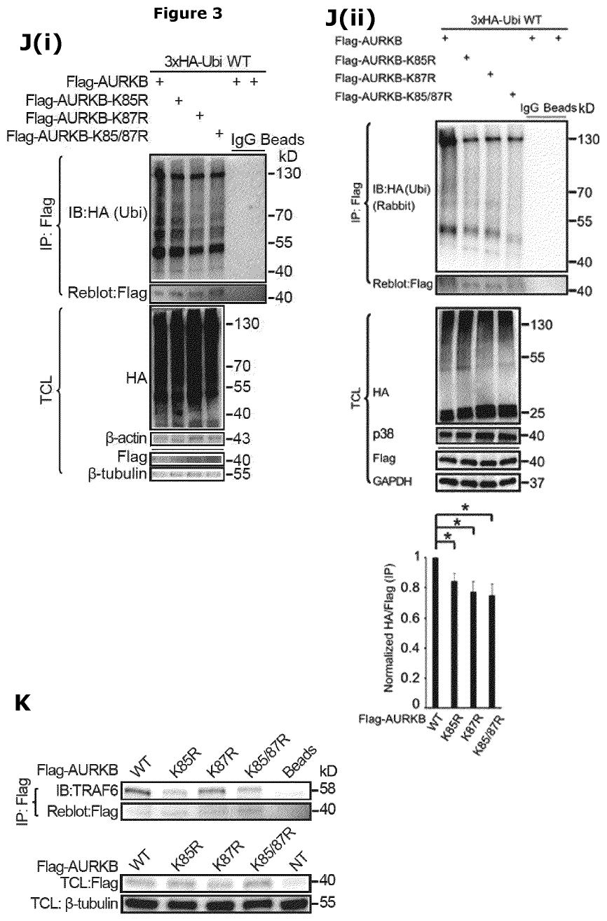

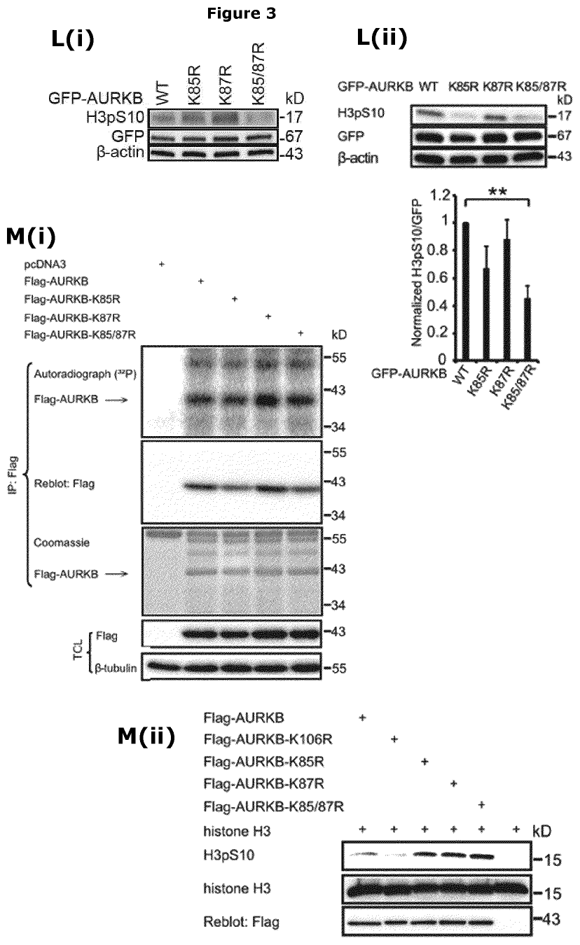

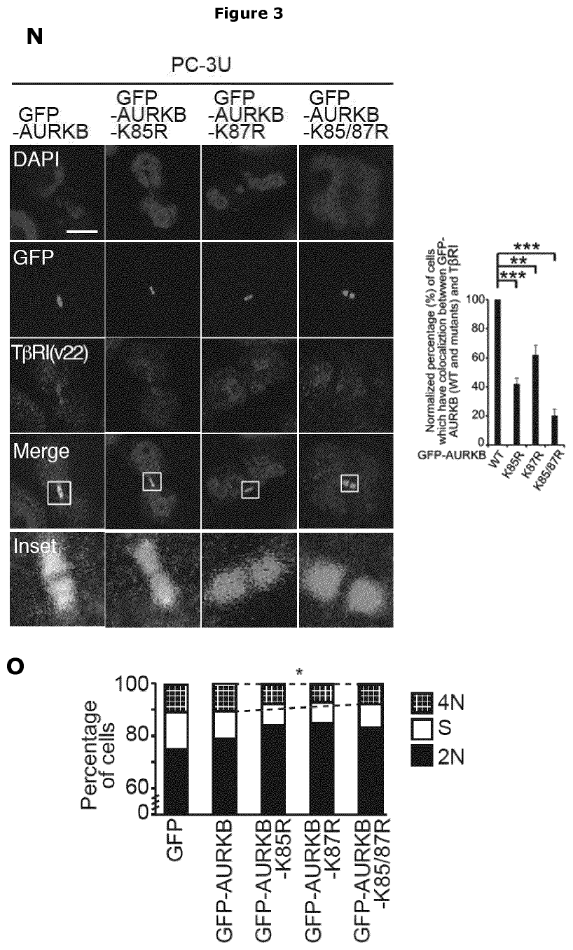

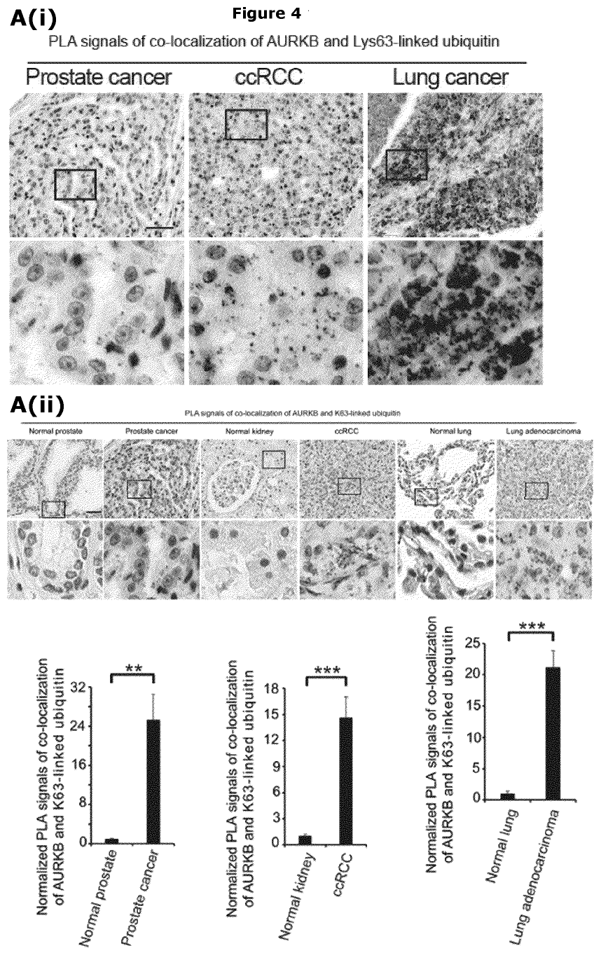

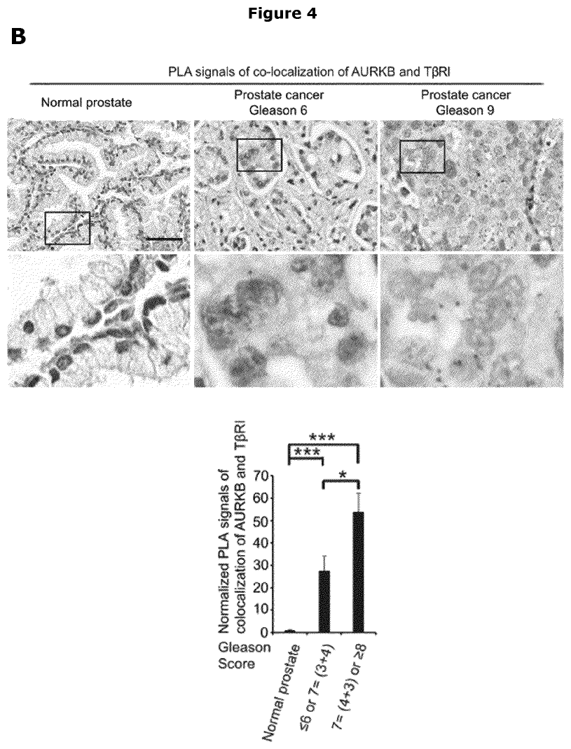

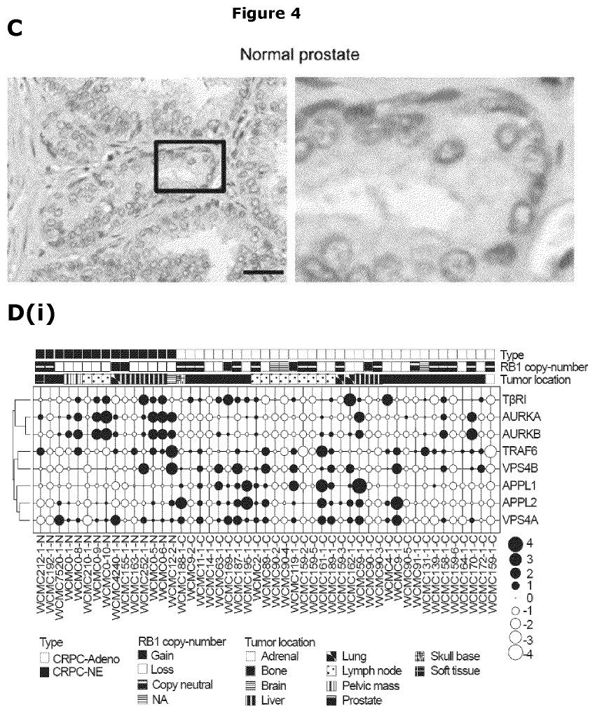

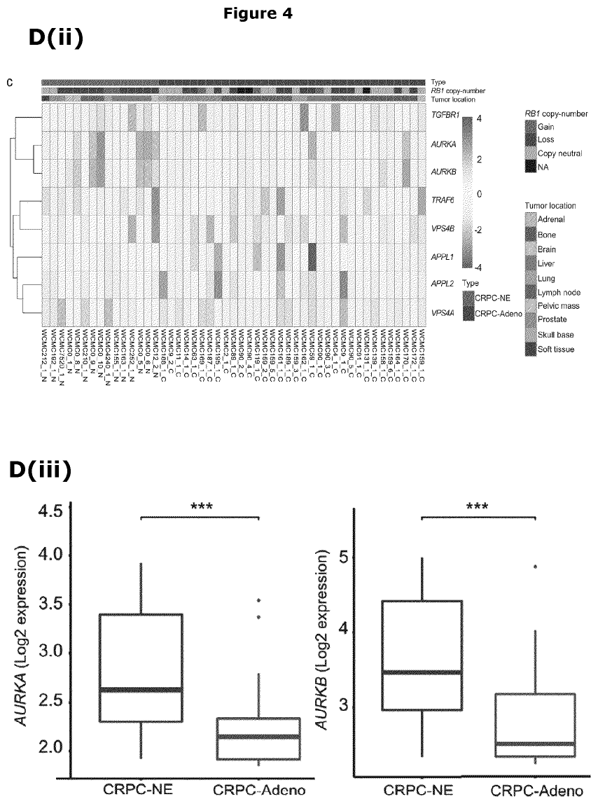

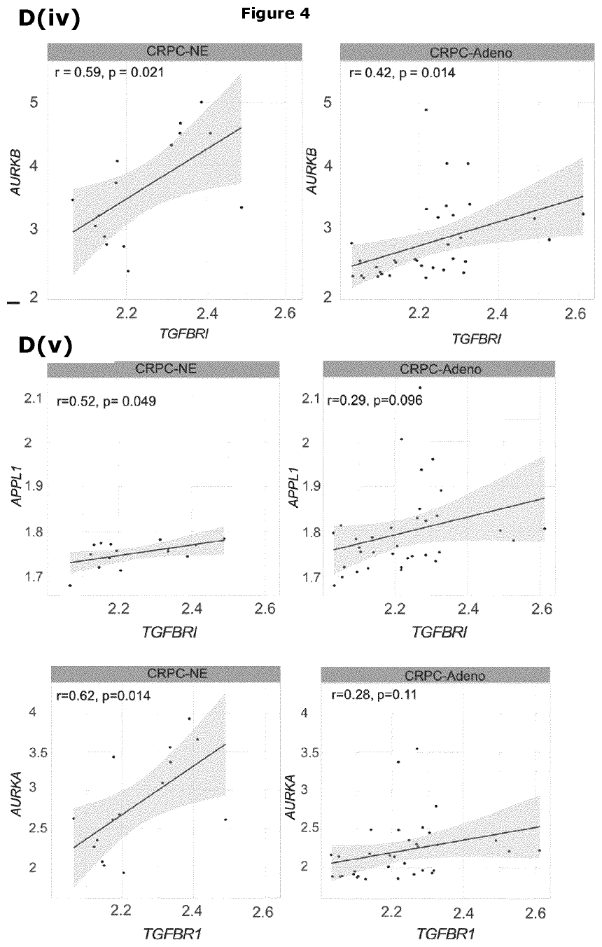

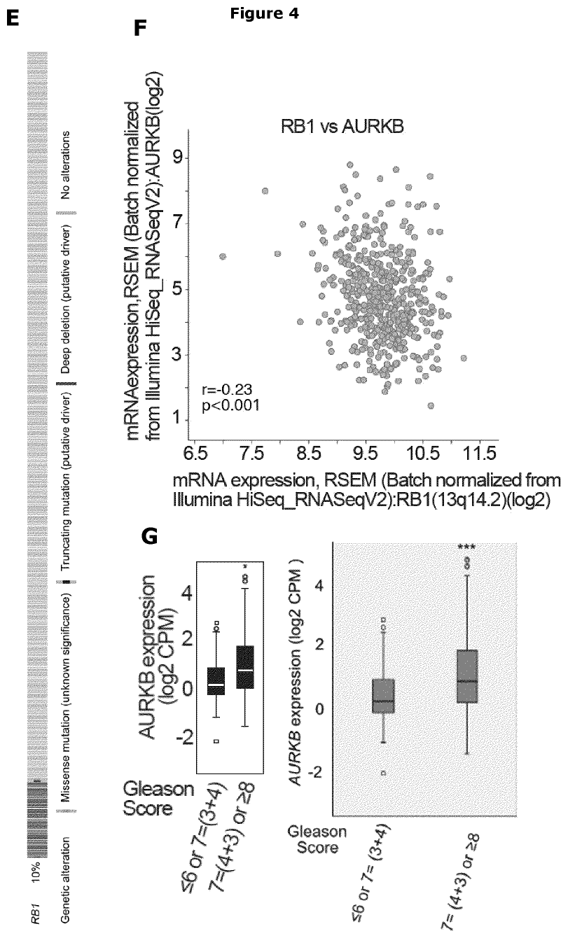

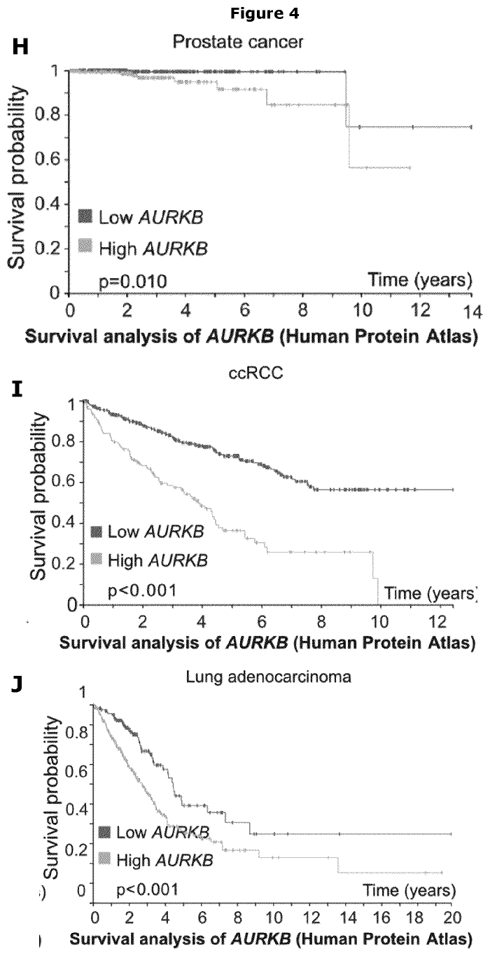

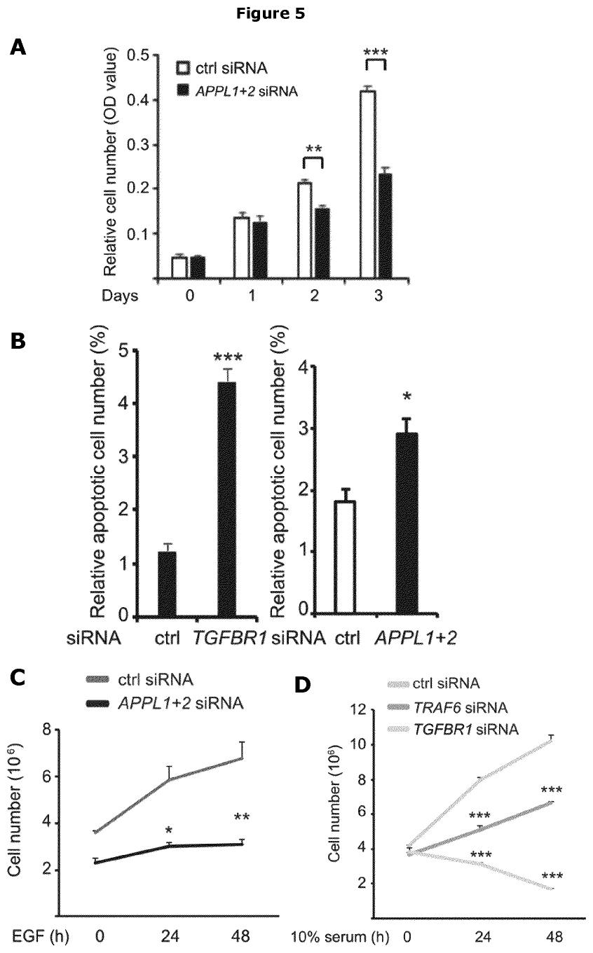

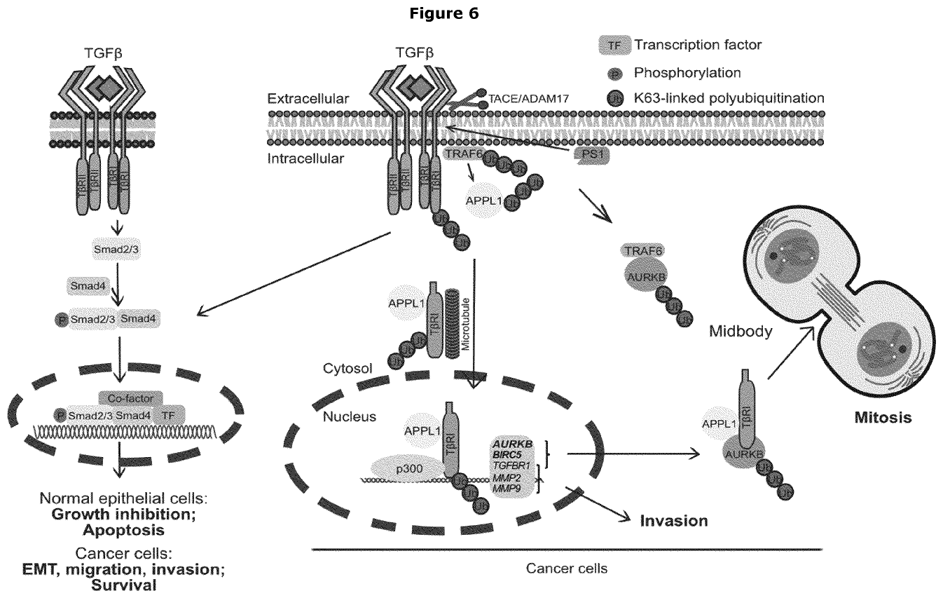

(A) Human prostate cancer PC-3U cells were transfected with control or No. 1 APPL1 and APPL2 SIRNA. RNA was extracted from cells, and microarray analysis was performed. (B(i) and (ii)) qRT-PCR analysis of the genes shown in panel a of cells treated with or without No. 1 APPLI and APPL2 siRNA. Inhibition by siRNA was overcome by expressing siRNA-resistant constructs; N=4, data presented as mean±SEM [Student's t-test, *P<0.05, **P<0.01, ***P<0.001]. (B (iii) and (iv)) qRT-PCR was performed to validate the microarray results of FIG. 1b using a second pair of siRNAS (No. 2; N=3). Data are presented as mean±SD [Student's t-test, ***P<0.001]. (C(i)) Expression of survivin and AURKB was evaluated by immunoblotting in PC-3U cells treated or not with No. 1 APPL1/2 siRNA and TGFβ. (C(ii)) PC-3U cells were synchronized with a double thymidine block and treated with No. 1 APPL1 and APPL2 siRNA. Cells were released and cell lysates were prepared at different times, and subjected to immunoblotting. (D) PC-3U cells were transfected with or without No. 1 APPL1 and APPL2 siRNA, incubated with nocodazole for 12 h, and analyzed by immunoblotting. (E) Immunofluorescence and confocal imaging showing co-localization of AURKB (green) and APPL1 (red) during telophase and cytokinesis. (F-K) Orthogonal views (XY, XZ and YZ) of two Z-stack images of panel e. (F, I) XY view (z-projection). (G, J) XZ view. (H, K) YZ view. Scale bar, 20 μm. (L) PC-3U cells were treated with TGFβ for different time periods; cell lysates were then subjected to immunoprecipitation using anti-survivin antibodies and immunoblotting using antibodies against APPL1 and TβRI. IB, immunoblot; TCL, total cell lysates. (M) PC-3U cells were transfected with full-length GFP-APPL1, yellow fluorescent protein (YFP)-APPL1-ΔN, or GFP-APPL1-ΔC and then stained with AURKB (red). The green channel was selected to show both GFP and YFP. Scale bar, 20 μm. A schematic representation of the APPL1 protein and mutants is included. (N) Schematic representation of the APPL1 protein and mutants. (O) PC-3U cells transiently transfected with HA-AURKB and different APPL1 domains as indicated, were synchronized and then subjected to immunoprecipitation with an antibody against HA and immunoblotting using a GFP antibody. Non-transfected (NT).

FIG. 2. TβRI co-localizes with AURKB during mitosis.

(A, B and D) Immunofluorescence experiments showing co-localization of AURKB (green) and TβRI (V22, red) during mitosis in human prostate cancer (PC-3U) (A) and human neuroblastoma (KELLY) (B) cells, and of TβRI (V22, green) and β-tubulin (red) throughout the PC-3U mitosis (D). Scale bar, 20 μm. (C) Localization of survivin (green) and TβRI (V22, red) in PC-3U cells throughout the mitosis (E) Decreased co-localization of TβRI and AURKB after treatment of PC-3U cells on ice for 30 min. Scale bar, 20 μm. (F) Representative confocal images showing the localization of green fluorescent protein (GFP)-VPS4A (green) and β-tubulin (red) with or without knockdown of TβRI by siRNA, or treatment with the TβRI kinase inhibitor SB505124. Scale bar, 5 μm. (G) Multinucleated cells were counted after knockdown of TGFBR1. Data presented as mean±SEM, N=3 [Student's t-test, *P<0.05]. Scale bar, 20 μm. (H) Gene Set Enrichment Analysis (GSEA) of genes ranked by their correlation with TGFBR1 expression yielded 34 significantly enriched gene sets (adjusted p-value≤0.05 and the p-values are adjusted using the Benjamini-Hochberg procedure). The ridge-plot shows the distribution of correlation coefficients of the core enriched genes, i.e., genes which contribute most to the enrichment of the gene set. The gene sets are ordered by normalized enrichment score. Color indicates the adjusted p-value. (I) GSEA plots of the hallmark mitotic spindle (left) and G2/M checkpoint (right) gene sets show their strong association with TGFBR1-correlated genes. The upper panels show the correlation coefficients and position of the gene set genes within the ranked list of all genes, and the lower panels show the running enrichment score. (J) PC-3U cells were treated or not with SB505124 and TGFβ for 30 minutes, after which cell lysates were analyzed by immunoblotting. (K) In vitro kinase assay showing that AURKB can phosphorylate TβRI. (L) PC-3U cells were stained with antibodies against p-Smad2 (red) and AURKB (green). Red and green scale bar, 5 μm; white scale bar, 20 pm.

FIG. 3. TRAF6 mediates K63-linked polyubiquitination of AURKB and the colocalization between AURKB and TβRI is dependent on TRAF6 and characteristics of mutants AURKB.

(A-B) PC-3U cells were treated with or without TRAF6 siRNA, synchronized with a double thymidine block and subjected to analysis by immunoblotting (IB) after different time periods (A), with or without incubation for 12 h with nocodazole (B). (C) Lysates of synchronized PC-3U cells were immunoprecipitated (IP) with an AURKB antibody, followed by immunoblotting with antibodies against TβRI, APPL1 and TRAF6. (D(i) and D(ii)) Lysates of synchronized PC-3U cells transfected with Flag-AURKB and HA-tagged wild-type (WT) or mutated ubiquitin, were subjected to immunoprecipitation using a Flag antibody, followed by immunoblotting using an HA antibody. Arrow points to heavy immunoglobulin chain. (E) PC-3U cells were synchronized with a double thymidine block, released to fresh media with 10% FBS, harvested at the indicated times, and then subjected to in vivo ubiquitination assay. S is short for starvation. (F) Lysates of synchronized PC-3U cells treated with or without TRAF6 siRNA were subjected to immunoprecipitation using a Flag antibody, followed by immunoblotting using an HA antibody. Arrow points to heavy immunoglobulin chain. Data presented as mean±SEM, N=3 [Student's t-test, **P<0.01] (G-H) Immunofluorescence showing reduced co-localization of AURKB and TβRI during mitosis when TRAF6 expression was decreased in PC-3U and MEF cells. (I) The consensus motif for ubiquitination by TRAF6 is present in AURKB in several species. Amino acids of the same type are labeled as (*) hydrophobic, (&) polar, (X) any amino acid residue. (K) is the acceptor lysine residue. (J((i) and (ii)) Lysates of synchronized PC-3U cells transfected with HA-tagged WT ubiquitin and Flag-tagged WT or mutant AURKB, were subjected to immunoprecipitation using a Flag antibody, followed by immunoblotting using an HA rabbit antibody. Data presented as mean±SEM, N=3 [Student's t-test, *P<0.05]. (K) Lysates of PC-3U cells transfected with Flag-tagged WT and mutant AURKB were immunoprecipitated with a Flag antibody and then subjected to immunoblotting with a TRAF6 antibody, as indicated. (L(i) and (ii)) PC-3U cells were transfected with WT or mutant GFP-AURKB, and then subjected to immunoblotting with an antibody against H3pS10. Data presented as mean±SEM, N=3 [Student's t-test, **P<0.01]. (M(i) and (ii)) Immunoprecipitated Flag-AURKB or its mutants were subjected to the in vitro kinase assays. The expression of Flag-AURKB and its mutants and equal loading was controlled by immunoblotting aliquots of the Flag immunoprecipitates or total cell lysate (TCL), as indicated. Incorporated radioactivity was detected by a phosphorimager. Migration positions of phosphorylated proteins and total proteins detected after staining of gels with Coomassie Brilliant Blue are shown by arrows (M(i)). Histone H3 was used as substrate and H3pS10 was detected by immunoblotting (M(ii)). (N) PC-3U cells were transfected with WT or mutant GFP-AURKB, then stained with TβRI (red). n=20, N=3, data presented as mean±SEM [Student's t-test, **P<0.01, ***P<0.001]. (O) PC-3U cells were transfected with WT or mutant GFP-AURKB, then stained with Hoechst 33342. N=3 [Student's t-test, *P<0.05].

FIG. 4. The expression of AURKB correlates with poor prognosis in different cancers and the relation between RB1, and AURKB expression in prostate cancer and correlation between the expression of APPL1, AURKA and TGFBR1 in CRPC, and AURKB is ubiquitinated in different cancers and forms a complex with TβRI in prostate cancer.

(A(i)) In situ PLA was performed on TMA to show the colocalization of AURKB and Lys63-linked polyubiquitin (brown dots). (A(ii)) In situ PLA was performed on TMAs to investigate the co-localization of AURKB and K63-linked ubiquitin (brown dots). The numbers of normal prostates, kidneys, and lungs were 22, 24, and 23, respectively. The numbers of prostate cancers, ccRCC, and lung adenocarcinoma were 41, 38, and 32, respectively. Quantification shows mean±SEM [Student's t-test, **P<0.01, ***P<0.001] (B) The association between AURKB and TβRI in prostate cancer TMA of patient materials (brown dots) was determined by in situ PLA. A total of 29 patients with low Gleason scores and 28 patients with high Gleason scores were included. The numbers of normal prostates were 23. Quantification shows mean±SEM [Student's t-test, *P<0.05, ***P<0.001]. Scale bar, 50 μm. (C) Lack of association between AURKB and TβRI in normal prostate tissue (brown dots), as determined by in situ PLA, serving as a negative control (no primary antibody was added). Scale bar, 50 μm. (D(i) and (ii)) Expression of eight genes of interest across 49 CRPC samples, with 15 CRPC-NE samples and 34 CPPC-Adeno samples, including both primary tumors and metastases. Samples are grouped first by their subtype and then by the tumor location. (D(iii)) The expression of AURKA and AURKB in CRPC-NE and CRPC-Adeno [Mann-Whitney U test, ***P<0.001]. (D(iv)) The expression of AURKB and TGFBR1 was correlated in both CRPC-NE and CRPC-Adeno. Pearson correlation analysis was used for data analysis. (D(v)) Correlation of APPL1, AURKA and TGFBR1 expression in CRPC-NE and CRPC-Adeno. Pearson correlation analysis are used for data analysis. (E) RB1 mutations in prostate cancer. (F) Negative correlation between expression of mRNA for AURKB and RB1 in prostate cancer; Pearson correlation coefficient (r) is presented. Data were obtained from cBioPortal TCGA PanCancer Atlas databases. (G) Expression of AURKB in the primary prostate tumors in TCGA differed between Gleason groups [Student's t-test, ***P<0.001]. Tumors were grouped based on their Gleason scores. (H-J) Kaplan-Meier plots illustrating the effects on the survival of patients of low vs. high expression of AURKB in prostate cancer, ccRCC, or lung adenocarcinoma. Representative images were obtained from the Human Protein Atlas, based on data from the TCGA Pan Cancer Atlas database.

FIG. 5. Effects of APPL1/2, TGFBR1 and TRAF6 on cell proliferation and survival.

(A) PC-3U cells were treated with control (ctrl) siRNA or No.1 APPL1/2 siRNA and subjected to MTT assay after different number of days in culture. (B) Apoptotic cells were counted among cells transfected with the different siRNAs. (C-D) Effects of silencing of the APPL1/2 genes in PC-3U cells on EGF stimulated cell growth (C), and of silencing of the TRAF6 or TGFBR1 genes with siRNA on cell number stimulated by 10% FBS (D). N=3, Quantification shows mean±SEM [Student's t-test, *P<0.05, P<0.01,***P<0.001].

FIG. 6. Schematic illustration of the TβRI-ICD signaling pathway and its involvement in mitotic progression.

The non-canonical pathway in which TβRI undergoes proteolytic cleavage by TACE/ADAM17 and presenilin 1 in the activated γ-secretase complex, generates an intracellular domain (TβRI-ICD). The endosomal protein APPL1/2 and intact microtubules are required for the nuclear translocation of TβRI-ICD. In the nucleus, TβRI-ICD forms a complex with the transcriptional co-activator p300 and promotes expression of pro-invasive genes, TGFBR1, as well as AURKB and BIRC5 (encoding survivin). During cell division, TβRI-ICD and APPLI form a complex with AURKB. TRAF6 promotes K63-linked polyubiquitination of AURKB on K85 and K87 during mitosis, which together with TβRI-ICD are required for proper cytokinesis.

It is to be understood that the present invention is not limited to the particular materials and methods described or equipment, as these may vary. It is also to be understood that the terminology used herein is for the purpose of describing particular embodiments only and is not intended to limit the scope of the present invention, which will be limited only by the appended claims.

It should be noted that as used herein and in the appended claims, the singular forms “a,” “an,” and “the” include plural reference unless the context clearly dictates otherwise. Thus, for example, a reference to “an antibody” is a reference to one or more antibodies and derivatives thereof known to those skilled in the art, and so forth. By “biomarker” we include the meaning of a naturally-occurring biological molecule, or component or fragment thereof, the measurement of which can provide information useful in the prognosis and/or diagnosis of cancer. For example, the biomarker may be a naturally-occurring protein or carbohydrate moiety, or an antigenic component or fragment thereof.

By “diagnosis” we include the meaning of determining the presence or absence of a disease state in an individual (e.g., determining whether an individual is or is not suffering from cancer, including an aggressive cancer).

The terms “cancer” and “cancerous” refer to or describe the physiological condition in mammals is typically characterized by unregulated cell growth. Examples of cancer include but are not limited to, carcinoma, prostate cancer, small cell lung cancer, kidney cancer, endometrial cancer, ovarian cancer, skin cancer and colorectal cancer. The grade score (numerical: G1 up to G4) increases with the lack of cellular differentiation—it reflects how much the tumor cells differ from the cells of the normal tissue they have originated from. Tumors may be graded on four-tier, three-tier, or two-tier scales, depending on the institution and the tumor type. The histologic tumor grade score along with the metastatic (whole-body-level cancer-spread) staging are used to evaluate each specific cancer subject, develop their individual treatment strategy and to predict their prognosis. The most commonly used system of grading is as per the guidelines of the American Joint Commission on Cancer. As per their standards, the following are the grading categories: GX Grade cannot be assessed; G1 Well differentiated (Low grade); G2 Moderately differentiated (Intermediate grade); G3 Poorly differentiated (High grade) and G4 Undifferentiated (High grade).

The terms “neoplasm” or “tumour” may be used interchangeably and refer to an abnormal mass of tissue wherein the growth of the mass surpasses and is not coordinated with the growth of normal tissue. A neoplasm or tumour may be defined as “benign” or “malignant” depending on the following characteristics: degree of cellular differentiation including morphology and functionality, rate of growth, local invasion and metastasis. A “benign” neoplasm is generally well differentiated, has characteristically slower growth than a malignant neoplasm and remains localized to the site of origin. In addition, a benign neoplasm does not have the capacity to infiltrate, invade or metastasize to distant sites.

A “malignant” neoplasm is generally poorly differentiated (anaplasia), has characteristically rapid growth accompanied by progressive infiltration, invasion, and destruction of the surrounding tissue. Furthermore, a malignant neoplasm has the capacity to metastasize to distant sites.

The term “prostate cancer” refers to a malignant neoplasm of the prostate within a given subject, wherein the neoplasm is of epithelial origin and is also referred to as a carcinoma of the prostate. Prostate cancer can be defined according to its type, stage and/or grade. Typical staging systems include the Jewett-Whitmore system and the TNM system (the system adopted by the American Joint Committee on Cancer and the International Union Against Cancer). A typical grading system is the Gleason Score which is a measure of tumour aggressiveness based on pathological examination of tissue biopsy.

The Gleason system is used to grade the adenocarcinoma cells in prostate cancer. This system uses a grading score ranging from 2 to 10, but scores below 6 are rarely used. A Gleason score is given to prostate cancer based upon its microscopic appearance. Cancers with a higher Gleason score are more aggressive and have a worse prognosis. Since prostate cancers often have areas with different grades, a grade is assigned to the two areas that make up most of the cancer. The Gleason score is based on the sum of two numbers: the first number is the grade of the most common tumor pattern; the second number is the grade of the second most common pattern. A pathologist examines the biopsy specimen and attempts to give a final Gleason score to the two patterns. Cancers with a Gleason score of 6 or less may be called well-differentiated or low-grade. Cancers with a Gleason score of 7 may be called moderately-differentiated or intermediate-grade. Cancers with Gleason scores of 8 to 10 may be called poorly-differentiated or high-grade.

The term “prostate cancer”, when used without qualification, includes both localized and metastasized prostate cancer. The term “prostate cancer” can be qualified by the terms “localized” or “metastasized” to differentiate between different types of tumour as those words are defined herein. The terms “prostate cancer” and “malignant disease of the prostate” are used interchangeably herein.

The term “differentiation” refers to the extent to which parenchymal cells resemble comparable normal cells both morphologically and functionally.

The term “metastasis” refers to spread or migration of cancerous cells from a primary (original) tumour to another organ or tissue and is typically identifiable by the presence of a “secondary tumour” or “secondary cell mass” of the tissue type of the primary (original) tumour and not of that of the organ or tissue in which the secondary (metastatic) tumour is located. For example, a prostate cancer that has migrated to bone is said to be metastasized prostate cancer and consists of cancerous prostate cancer cells in the prostate as well as cancerous prostate cancer cells growing in bone tissue.

The terms “a non-malignant disease of the prostate”, “non-prostate cancer state” and “benign prostatic disease” may be used interchangeably and refer to a disease state of the prostate that has not been classified as prostate cancer according to specific diagnostic methods including but not limited to rectal palpitation, PSA scoring, transrectal ultrasonography and tissue biopsy. Such diseases include, but are not limited to, an inflammation of prostatic tissue (i.e., chronic bacterial prostatitis, acute bacterial prostatitis, chronic abacterial prostatitis) and benign prostate hyperplasia.

The term “healthy” refers to an absence of any malignant or non-malignant disease; thus, a “healthy individual” may have other diseases or conditions that would normally not be considered “healthy”. A “healthy” individual demonstrates an absence of any malignant or non-malignant disease.

In the context of prostate cancer, the term “healthy” refers to an absence of any malignant or non-malignant disease of the prostate; thus, a “healthy individual” may have other diseases or conditions that would normally not be considered “healthy”. A “healthy” individual demonstrates an absence of any malignant or non-malignant disease of the prostate.

By “cytokinesis” we include the meaning of the physical process of cell division during which the cytoplasm of a parental eukaryotic cell divides into two daughter cells. It occurs concurrently with two types of nuclear division called mitosis and meiosis, which occur in animal cells. Mitosis result in two separate nuclei contained within a single cell. Cytokinesis performs an essential process to separate the cell in half and ensure that one nucleus ends up in each daughter cell. Cytokinesis starts during the nuclear division phase called anaphase and continues through telophase. A ring of protein filaments called the contractile ring forms around the equator of the cell just beneath the plasma membrane. The contractile ring shrinks at the equator of the cell, pinching the plasma membrane inward, and forming what is called a cleavage furrow. Eventually, the contractile ring shrinks to the point that there are two separate cells each bound by its own plasma membrane. Abscission, the process through which the membrane connecting the two newly generated cells is severed resulting in physical separation of the siblings, concludes cytokinesis.

By “midbody” we include the meaning of a transient structure that connects two daughter cells at the end of cytokinesis, with the principal function being to localize the site of abscission, which physically separates two daughter cells. The midbody forms from the midzone, which is a bipolar microtubule array that assembles between separating sister chromatids during anaphase. After the cleavage furrow is formed, the central spindle midzone is reconstructed to form a midbody. The midbody provides an important platform for recruiting and organizing crucial proteins that regulate the detachment of two daughter cells Conversion of midzones to midbodies correlates positively with furrow ingression.

By the term “co-localized” we include the meaning of the presence of two or more molecules/proteins/compounds/biomarkers at the same cellular location, for example to the midbody. As used herein by the terms “associated” and “co-localized” we include that these molecules/compounds/proteins/biomarkers compounds are spatially and temporally localized to the same region of a cell, but not necessarily in a complex in which each component directly interacts with each other.

Co-localization of biomarkers in a cytokinesis structure can be determined by methods known in the art and includes those described herein.

The term “presence,” “expression”, “level”, “amount”, and “expression level” may relate to the amount of a nucleic acid molecule, such as DNA and mRNA, and/or a protein of a defined biomolecule, such as for example APPL1, AURKB, TβRI, TβRI-ICD and TRAF6. AURKB may also be ubiquitinated. The level of each biomolecule is determined at a specific and predetermined site in a cell, such as for example the nucleus, the cytosol, cell-membrane, cytokinesis structure etc.

AURKB can be non-ubiquitinated, ubiquitinated, polyubiquitinated.

The term event means any change in the method of treatment, such as start, change of medication and finalizing a treatment. A change in the levels of the biomarkers, i.e., Aurora kinase B (AURKB), Adaptor Protein, Phosphotyrosine Interacting With PH Domain And Leucine Zipper 1 (APPL1), TGFβ receptor type 1 (TβR1) and TNF receptor associated factor 6 (TRAF6), co-localized in the cytokinesis structure which provides information regarding a disease or condition involving abnormal cleavage of transforming growth factor β type I receptor (TβRI) or a treatment of said related disease or condition.

In this specification, an antibody (V22) binding to (for detecting and/or visualizing) the intracellular domain of TGFβ receptor type 1 (TβR1-ICD) was used. However, even if TβR1-ICD (about 34 kDa) is detected and visualized in a sample it does not mean that only this domain is present, it can be, but V22 binds also to the intact full-length protein (55.96 kDa), hence also recognized by V22. To evaluate if only the ICD or for example the full-length protein is present, the respective molecular weight may for example be determined.

This means that the term “TGFβ receptor type 1 (TβR1)” as a biomarker may in some embodiments mean the intracellular domain (TβR1-ICD).

A reference value means a value representing an expression level, such as the amount (for example mRNA or protein) or intensity (e.g. immunofluorescence and other imaging methods, Western blot) of respective biomarker, such as APPL1, AURKB, TβRI, TβRI-ICD and TRAF6 in a biological sample. The sample may be a biopsy taken from a solid tumour, benign or malign, for defining a start/reference value t₀ for use as a reference point and detect how time and/or a change in for example medication, dose, time, addition of medication (and combinations) etc. affects the reference value t_{1,2,3etc}, positively or negatively. A non-cancerous tissue exhibits a reference value of 0, i.e., the markers (APPL1, AURKB, TβRI, and TRAF6) are not co-localized during cytokinesis. The term inhibitor or blocker means an agent or compound that binds to a protein/enzyme and thereby decrease the protein/enzyme activity, or physically blocks a site on a protein, membrane, cell etc. thereby sterically hinder other agents to reach that site.

The present invention provides reliable biomarkers for selecting/classifying subjects suffering from a cancer associated with the non-canonical TGFβ-induced signaling pathways involving cleavage of transforming growth factor β type I receptor (TβRI), predicting response to treatments, monitoring the outcome of treatments with an inhibitor/blocker for cleavage of TβRI provides valuable tools for successful cancer treatments.

Moreover, the present invention is also responding to the unmet high medical need for identifying invasive cancer growth and thereby preventing metastasis in an early phase of the disease.

### Material and Methods

### Cell Culture

The human prostate cancer cell line PC-3U (RRID:CVCL_0482) and the human neuroblastoma cell line KELLY which were purchased from Sigma (RRID: CVCL_2092) were grown in RPMI-1640 supplemented with 10% fetal bovine serum (FBS), 2 mM L-glutamine, and 100 units/ml penicillin and 0.1 mg/ml streptomycin. Immortalized wild-type mouse embryonic fibroblasts (MEFs) or MEFs deficient in TRAF6 (from Jun-ichiro Inoue) were grown in Dulbecco's modified Eagle's medium containing 10% FBS, 4 mM L-glutamine, and 100 units/ml penicillin and 0.1 mg/ml streptomycin. For TGFβ stimulation experiments, TGFβ (5 ng/ml) was prepared in medium containing 1% FBS and added to cells that had been starved for 18 h in RPMI medium supplemented with 1% FBS. Transient transfection was performed with FuGENE HD (Roche) according to the manufacturer's instructions. The cell lines have been validated by IDEXX BioAnalytics.

### Antibodies and Reagents Used for Immunoblotting

Antibodies against the following proteins were used for immunoblotting: APPL1 (Cell Signaling Technology Cat #3858, RRID:AB_2056989), p-Aurora kinases (Thr288 in AURKA; Thr232 in AURKB; Thr198 in AURKC; the molecular masses of the proteins are 48 kDa, 40 kDa, and 35 kDa, respectively) (Cell Signaling Technology Cat #2914, RRID: AB_2061631), B1 cyclin (Cell Signaling Technology Cat #4135, RRID: AB_2233956), HA (Cell Signaling Technology Cat #3724, RRID: AB_1549585 and Cell Signaling Technology Cat #2367, RRID: AB_10691311), GFP (Cell Signaling Technology Cat #2956, RRID: AB_1196615), GAPDH (Cell Signaling Technology Cat #5174, RRID: AB_10622025), p38 (Cell Signaling Technology Cat #8690, (Cell Signaling Technology RRID:AB_10999090), survivin Cat #2808, Cruz Biotechnology Cat # sc-67403, RRID: AB_2063948), APPL2 (Santa RRID: AB_2056383), AURKB (Abcam Cat # ab2254, RRID: AB_302923), TRAF6 (Abcam Cat # ab40675, RRID: AB_778573), Flag (Sigma-Aldrich Cat # F9291, RRID: AB_439698), β-actin (Sigma-Aldrich Cat # A5441, RRID: AB_476744), β-tubulin (Sigma-Aldrich Cat # T0198, RRID:AB_477556 and Cell Signaling Technology Cat #2146, RRID:AB_2210545), H3pS10 (Millipore Cat #06-570, RRID:AB_310177), and TβRI (V22; Santa Cruz Biotechnology Cat # sc-398, RRID: AB_632493; this antibody specifically recognizes the ICD of TβRI, as described before13). Horseradish peroxidase-coupled secondary antibodies were purchased from Dako and Protein-G Sepharose and ECL immunoblotting detection reagents from GE Healthcare. Pefabloc was obtained from Roche, PageRuler Prestained Protein Ladder was from Thermo Fisher Scientific.

### Protein Analysis

Cells were washed twice with ice-cold phosphate-buffered saline (PBS) and lysed in ice-cold lysis buffer [150 mM NaCl, 50 mM Tris, pH 8.0, 0.5% (v/v) deoxycholate, 1% (v/v) NP40, 10% (v/v) glycerol and protease inhibitors]. After centrifugation, the supernatants were collected, and protein concentrations determined using the BCA Protein Assay Kit (ThermoFisher Scientific). Equal amounts of protein from each total cell lysate were used for immunoprecipitation. Immunoprecipitated proteins were resolved by sodium dodecylsulfate (SDS)-polyacrylaminde electrophoresis (PAGE) on Mini-PROTEAN TGX gels (Bio-Rad) blotted onto nitrocellulose membranes, and subjected to immunoblotting as described previously (Song J, Mu Y, Li C, Bergh A, Miaczynska M, Heldin C-H, et al. APPL proteins promote TGFβ-induced nuclear transport of the TGFβ type I receptor intracellular domain. Oncotarget. 2016; 7:279-92).

### In Vivo Ubiquitination Assay

PC-3U cells were washed once in ice-cold PBS, collected in 1 ml ice-cold PBS, and then centrifuged at 300×g for 5 min at 4° C. Noncovalent protein interactions were dissociated in fresh-made 1% SDS in PBS and by boiling for 10 min. Samples were diluted in 1.5 ml lysis buffer containing 0.5% NP-40 with protease inhibitors in PBS. The samples were subjected to immunoprecipitation, followed by immunoblotting, as described previously (Hamidi A, Song J, Thakur N, et al. TGF-β promotes PI3K-AKT signaling and prostate cancer cell migration through the TRAF6-mediated ubiquitylation of p85α. Sci Signal 2017; 10: eaal4186).

### Immunofluorescence and Microscope Image Acquisition

Other primary antibodies against the following proteins were used in immunofluorescence experiments: AURKB (Novus, Cat # NBP2-50039, RRID: AB_2895237), and p-Smad2 (Cell Signaling Technology Cat #3108, RRID: AB_490941). Secondary antibodies were: donkey anti-rabbit Alexa Fluor 555 (Thermo Fisher Scientific Cat # A-31572, RRID:AB_162543), donkey anti-mouse Alexa Fluor 555 (Thermo Fisher Scientific Cat # A-31570, RID:AB_2536180), and goat anti-mouse Alexa Fluor 488 (Thermo Fisher Scientific Cat # A-11029, RRID: AB_2534088), and goat anti-rabbit Alexa Fluor 488 (Thermo Fisher Scientific Cat # A32731, RRID:AB_2633280). Immunofluorescence assays were performed as described previously (Song J, et al. Oncotarget 2016; 7: 279-292). Briefly, cells were plated on coverslips, fixed in 4% paraformaldehyde for 30 min, and then treated with 0.2% Triton X-100 in PBS for 5 min and blocked with 10 mM glycine. Incubation with primary antibodies was performed for 1 h at room temperature, followed by washing in PBS and incubation with secondary antibodies. Photomicrographs were obtained using a confocal microscope LSM 710 (Carl Zeiss) with a 63×/1.4 NA objective lens (Carl Zeiss). The images were acquired under oil immersion at room temperature, using Zen 2011 software.

### Plasmids and Site-Directed Mutagenesis

pCR3-Flag-AURKB K106R kinase dead (KD) was a kind gift from Susanne Lens (Addgene Plasmid #108488; http://n2t.net/addgene:108488; RRID: Addgene_108488)⁴⁴ and was used for context optimization and to generate a construct expressing the Flag-tagged wild-type AURKB protein by QuickChange Lightning MultiSite-Directed Mutagenesis kit (Agilent Technologies). The primers for mutagenesis were oJS5, oJS8, oJS17, and oJS18 (Table 2). Plasmids expressing altered Flag-AURKB (i.e., K85R, K87R, and K85R/K87R double mutant) were generated by PCR mutagenesis, using oligo oJS9, oJS10, and oJS11, respectively. Similar approaches were employed to construct plasmids expressing the enhanced green fluorescent protein (EGFP)-fused to the wild-type AURKB, as well as the K85R, K87R, and K85R/K87R mutants, using pEGFP-AURKB K106R (KD) as the template for mutagenesis (Addgene Plasmid #108493; http://n2t.net/addgene:108493; RRID: Addgene_108493).44 Integration of tags and alterations of AURKB sequences were confirmed by DNA sequencing of the individual plasmids.

Plasmids carrying 6His-APPL1 and 6His-APPL2 (purchased from Thermo Fisher Scientific), were used as templates for mutagenesis to generate constructs producing transcripts that were tolerant of siRNA-induced gene silencing. The sequence of siRNA-resistance construct of APPL1 was 5′-AGAGAGATGGATTCAGACATA-3′ (SEQ ID NO: 3), and the sequence of siRNA-resistance construct of APPL2 was 5′-CAGATTTATCTCACAGATAAC-3′(SEQ ID NO:4). Alterations in APPL1 and APPL2 sequences were confirmed by DNA sequencing. YFP-APPL1-ΔN and GFP-APPL1-ΔC were kind gifts from Marta Miaczynska.⁴⁵ pEGFPC1-human APPL1 was a gift from Pietro De Camilli (Addgene plasmid #22198; http://n2t.net/addgene: 22198; RRID:Addgene_22198)⁴⁶ and was used to generate constructs harboring BAR domain, PH domain, and PTB domain respectively, by QuickChange Lightning MultiSite-Directed Mutagenesis kit (Agilent Technologies). The primers for mutagenesis were oYZ86, oYZ87, oYZ88, oYZ91 and oYZ92 (Table 2). Alterations of APPLI sequences were confirmed by DNA sequencing of the individual plasmids.

TABLE 2
The primers used in this study to generate
AURKB and APPL1 plasmids are shown.

| | | SEQ |
|---|---|---|
| | | ID |
| Name | Sequences | NO |
| oJS5 | CCCCGGGAATCAAAACGAATTCGCCACCATGG | 5 |
| oJS8 | CGAATTCGCCACCATGGACTACAAAGACGATGACGACA | 6 |
| | AGGCCCAGAAGGAGAACT | |
| oJS17 | TGAAGAGGACCTTGAGCGCCACGATGAAATGGC | 7 |
| oJS18 | CGCTTCTGTGCCCATGGGAGCCCAGG | 8 |
| oJS9 | CAAACTTGCCTCTGCCCAGAGGACGCCC | 9 |
| oJS10 | GTACACGTTTCCAAACCTGCCTTTGCCCAGAGG | 10 |
| oJS11 | ACACGTTTCCAAACCTGCCTCTGCCCAGAGGACGC | 11 |
| oYZ86 | ACCCCACCAAATTTCCTGAATTCTGCAGTCGACG | 12 |
| oYZ87 | CGGACTCAGATCTCGAGTGGTTAATCGAAATTTAACCC | 13 |
| | G | |
| oYZ88 | CTGTACAATAAACAACATATCTAAACAAATAGAATTCT | 14 |
| | GCAGTCGACGG | |
| oYZ91 | GTCCGGACTCAGATCTCGAGTGATTCTTCATCAGTTAT | 15 |
| | TTATTGT | |
| oYZ92 | GGATCGTAGGGCATCAGAATTCTGCAGTCGAC | 16 |

siRNA Transfection

On TARGET plus APPL1 (No. 1: target sequence, 5′-GGAAAUGGACAGUGAUAUA-3′ (SEQ ID NO: 17); No. 2: target sequence, 5′-GAUCUGAGUCUACAAAUUU-3′) (SEQ ID NO: 18), APPL2 (No. 1: target sequence, 5′-AGAUCUACCUGACCGACAA-3′ (SEQ ID NO: 19); No. 2: target sequence, 5′-GCGGAAAAGAUGCGGGUGU-3′) (SEQ ID NO:20), TβRI siRNA (target sequence, 5′-CAUAUUGCUGCAACCAGGA-3′) (SEQ ID NO:21), SMART pool TRAF6 SIRNA, and siGENOME non-targeting control siRNA #1 (target sequence, 5′-UAGCGACUAAACACAUCAA-3′) (SEQ ID NO:22) were obtained from Dharmacon Research. siRNA was transfected into cells using Oligofectamine Transfection Reagent (ThermoFisher Scientific), according to the manufacturer's protocol.

### Total RNA Extraction and Microarray Assay

After knockdown of APPL1 and APPL2, total RNA was extracted from PC-3U cells using the RNeasy Mini Kit (Qiagen). RNA purity and integrity were evaluated with the Agilent RNA 6000 Nano Kit and Agilent 2100 Bioanalyzer (Agilent Technologies). Total RNA (500 ng) was used to generate a biotin-labeled antisense RNA target with the TargetAmp™-Nano Labeling Kit for Illumina Expression Beadchip (Epicenter) following the manufacturer's protocol. RNA (750 ng) was hybridized to an Illumina Human HT-12 Beadchip array for 17 h. The chips were washed and stained with Cy3-streptavidin according to the manufacturer's instructions. Image data were acquired using the iScan system (Illumina). Microarray data were analyzed using GenomeStudio and DAVID Bioinformatics Resources 6.7 and verified by qRT-PCR.

### In Vitro Kinase Assay

For in vitro kinase assay, HEK293T cells were transfected with vectors for Flag-tagged AURKB or its mutants K85R, K87R, and K85/87R, or the control empty pcDNA3 vector, using FuGENE® HD (Promega). Proteins were extracted in RIPA lysis buffer (150 mM NaCl, 0.1% Triton X-100, 0.5% sodium deoxycholate, 0.1% SDS, 50 mM Tris-HCl, pH 8.0, protease inhibitors (Roche)) and immunoprecipitated with anti-Flag antibody (Sigma-Aldrich Cat # F1804, RRID:AB_262044) and protein G Sepharose (Invitrogen). The beads were washed four times in RIPA buffer, and equilibrated in kinase buffer (15 mM MOPS, pH 7.2, 7.5 mM glycerol 2-phosphate, 15 mM MgCl₂, 3 mM EGTA, 0.15 mM dithiothreitol).

The phosphorylation reaction was initiated by addition of substrate, histone H3 (1 μg) and ATP. In non-radioactive kinase assays, the concentration of ATP was 0.5 mM, while it was 5 μm in assays with 0.5 μCi [γ-32P] ATP (Perkin Elmer). For analyses on SDS-PAGE, reactions were stopped by addition of one-fifth volume of 6× SDS sample buffer, heated at 96° C. for 5 min and applied onto SDS-PAGE.

Phosphorylation of histone H3 was detected by immunoblotting with anti-phospho-histone H3 (Ser10) antibody (Millipore Cat #06-570, RRID:AB_310177). Equal expression and loading were controlled by immunoblotting of the membranes with anti-histone H3 antibody (Cell Signaling Technology Cat #4499, RRID:AB_10544537) and with anti-Flag antibody (Sigma-Aldrich Cat # F1804, RRID: AB_262044).

### Evaluation of Cell Number and Death

Cell number was measured using the Cell Proliferation Kit I (MTT) from Roche or automated cell counter Countess™ from Thermo Fisher Scientific. Cell apoptosis was analyzed using Arthur™ after staining with the Tali™ apoptosis kit (ThermoFisher Scientific).

### In Situ Proximity Ligation Assay (PLA)

For PLA brightfield, the prostate cancer tissue microarray (TMA; BioCat) was first deparaffinized, and then subjected to antigen retrieval, and permeabilization. PLA was performed using antibodies against AURKB (Novus Biologicals Cat # NBP2-50039, RRID:AB_2895237)), K63-linked polyubiquitin (Abcam Cat # ab179434, RRID: AB_2895239) and TβRI (V22, Santa Cruz Biotechnology Cat # sc-398, RRID: AB_632493) with Duolink Detection for Brightfield (Sigma). Images were acquired with Pannoramic 250 Flash, and PLA signals were analyzed using Duolink Image Tool software.

### Bioinformatics

Genes correlating with TGFβR1 in prostate cancer were identified by calculating Pearson's correlation coefficients using log2 CPM normalized expression data of the TCGA PRAD cohort. All genes were ranked by their correlation to TGFBR1, and Gene Set Enrichment Analysis (GSEA) was performed using the R package clusterProfiler⁴⁷ with the Hallmark gene sets of the Molecular Signatures Database (MSigDB)⁴⁸. 34 gene sets were enriched with an adjusted p-value of 0.05 or below.

RNA-seq expression data and clinical metadata from The Cancer Genome Atlas were downloaded using the Genomic Data Commons⁴⁹ and the R package TCGAbiolinks⁵⁰, v. 2.16.4. The primary and secondary Gleason grades for each prostate tumor were obtained from the file PRAD_clindata.xls. Tumors were grouped based on their Gleason scores. The log2 CPM (counts-per-million) normalized expression values of each gene of interest were plotted per Gleason group using the R package ggpubr⁵¹. The statistical significance of the expression difference was calculated using t-tests.

RNA-seq expression data and copy-number data for samples from 49 castration-resistant prostate cancer (CRPC) samples from a published study⁵² were downloaded from The cBio Cancer Genomics Portal (http://cbioportal.org). Clinical data were obtained from the file data_clinical_sample.txt and expression data fromdata_RNA_Seq_expression_median.txt and the copy-number data from data_log2CNA.txt. The data were read and subjected to all further analysis using R, v. 4.0.2⁵³. The expression data were log2-transformed, and a row-normalized heatmap was plotted with the samples sorted by subtype and tumor location, and genes hierarchically clustered by their expression profile. The RB1 copy-number status was defined as gain for an RB1 log2 copy-number value of 0.4 or above, as a loss for a value of −0.4 or below, and copy neutral otherwise. Copy-number data was unavailable for three adenocarcinoma samples. The expression difference of the genes of interest in neuroendocrine vs adenocarcinoma CRPC groups along with Mann-Whitney U test p-values were visualized with box plots generated by the ggboxplot function of the R package ggpubr⁵¹. Pearson's correlation between the expression of TGFBR1 and other genes were calculated within neuroendocrine CRPC samples and adenocarcinoma CRPC samples.

### Statistical Analysis

The Student's t-test or Mann-Whitney U test were used to analyze differences between two independent groups as indicated in the figure legends. Values are expressed as the mean±standard error of the mean (SEM) or ±standard deviation (SD) of at least three independent experiments. P values less than 0.05 were considered statistically significant. *P<0.05, **P<0.01, ***P<0.001.

### EXAMPLES

### Example 1. APPL Proteins Regulate Genes Involved in Proliferation and Apoptosis

The inventors found that the endosomal adaptor proteins Adaptor Protein, Phosphotyrosine Interacting With PH Domain And Leucine Zipper 1 (APPL1) and APPL2 are required for the nuclear accumulation of TβRI-ICD in response to TGFβ stimulation of cells¹⁶. To investigate the target genes of the nuclear TβRI-ICD-APPL1 complex, the inventors performed microarray analyses to assess the effect on gene expression of knocking down APPL1/2 (Table 1 and FIG. 1a).

Among the affected genes in APPL1/2 knockdown cells, it was observed decreased expression of genes encoding proteins involved in cell proliferation and apoptosis, i.e., components of the CPC [AURKB, survivin (encoded by BIRC5), and borealin (encoded by CDCA8)] and their downstream substrate, mitotic centromere-associated kinesin (encoded by KIF2C) (Table 1 and FIG. 1a).^{33,34,54}. No effect was observed on expression of INCENP.

TABLE 1
Regulated genes in siControl vs siAPPL1 + 2

| Gene name | Gene ID | Fold change |
|---|---|---|
| aurora kinase B | AURKB | 1.726 |
| baculoviral IAP repeat containing 5 | BIRC (survivin) | 1.451 |
| cell division cycle associated 8 | CDCA8 (borealin) | 1.235 |
| Kinesin family member 2C | KIF2C (MCAK) | 1.466 |

The inventors verified the microarray data using quantitative real-time PCR (qRT-PCR). Specifically, it was confirmed that the expression of AURKB, BIRC5, CDCA8, and KIF2C was decreased in cells transfected with two different APPL1/2 small interfering (si)RNAs (FIGS. 1B(i), (ii), (iii) and (iv)). Re-expression of the wild-type APPL1/2 protein from SiRNA-resistant constructs overcame the inhibition by APPL1/2 siRNA to a significant extent (FIG. 1B).

Since AURKB functions in the CPC complex, we determined the level of proteins and protein-protein interactions during mitotic progression of cells grown in 10% FBS or as specified below. Using immunoblotting, it was observed reduced AURKB and survivin protein levels in the APPL1/2 knockdown cells (FIG. 1C(i)). To examine whether the AURKB expression level is related to APPL1/2 proteins, a double thymidine block was used to synchronize PC-3U cells and then release them into the normal medium with 10% FBS to follow cell cycle progression. When the cells were treated with APPL1/2 SIRNA, expression of AURKB and phosphorylation of its substrate, histone H3 at Ser10 (H3pS10), was dramatically decreased in the cell cycle (FIG. 1C(ii)). The expression of cyclin B1 and TβRI was notably decreased after silencing the expression of APPL1/2 (FIG. 1C(ii)). The reduced expression of TβRI in cells treated with APPL1/2 siRNA is consistent with previous reports that nuclear TβRI-ICD promotes its own expression^{14,55}. In confirmation of these findings, cells that were arrested at the G2/M phase by nocodazole treatment also showed decreased levels of AURKB and H3pS10 (FIG. 1F). Interestingly, using immunofluorescence microscopy and z-stack imaging analyses, it was observed that APPL1 co-localized with AURKB in the cytokinetic structure (e.g. midbody) (FIG. 1E-K). Furthermore, a co-immunoprecipitation assay showed that APPL1 formed a complex with survivin in a TGFβ-dependent manner, peaking at 48 h (FIG. 1L).

The inventors previously reported that APPL1 is required for nuclear accumulation of TβRI-ICD and that the C-terminal part of APPL1 binds to TβRI 16. On the basis of these findings, the inventors investigated the effect of N- and C-terminal deletions of APPL1 on the levels of AURKB. Indeed, the expression of C-terminal deletion APPL1 mutant suppressed the level of AURKB, whereas an N-terminal deletion mutant did not have such an effect (FIG. 1M). Moreover, by co-immunoprecipitation experiments, we found that AURKB associated with all three domains of APPL1, including BAR domain, PH domain and PTB domain (FIGS. 1N-O).^{45,56} Taken together, these data support the notion that APPL1 associates with and regulates the expression of AURKB and that the expression of TGFBR1, which is dependent on nuclear TβRI-ICD 14,55, is cell cycle dependent.

### Example 2. TβRI Associates With AURKB in the Cytokinetic Structure During Mitosis

The inventors observed that APPL1 interacts with AURKB (FIGS. 1E-K and 1O) and forms a complex with TβRI. Since the expression of TβRI is cell cycle dependent, it was investigated whether TβRI also associates with CPC during mitosis and cytokinesis. Immunostaining experiments performed in PC-3U prostate cancer cells and KELLY neuroblastoma cells revealed that TβRI co-localized with AURKB in a cytokinetic structure (the midzone as well as in the midbody) (FIGS. 2A and B). A partial co-localization was detected between TβRI and survivin during telophase (FIG. 2C), and TβRI and β-tubulin clearly co-localized in the cytokinetic structure (midbody) (FIG. 2D). APPL1 has been reported to transport TβRI-ICD from endosomes to the nucleus via microtubules. Therefore, it was investigated whether intact microtubules are important for the TβRI localization; no interaction between AURKB and TβRI was seen in the cytokinetic structure when microtubules were depolymerized by cold treatment (FIG. 2E). Dynamic microtubules were also important for localization of AURKB during anaphase, which is consistent with a previous report⁵⁸. Of note, silencing the expression of TβRI resulted in abnormal abscission in around 42% of cytokinesis cells, but inhibition of the kinase activity of TβRI by SB505124 did not affect the abscission (FIG. 2F). Furthermore, knockdown of TβRI led to multinucleation (FIG. 2G), giving further support to the possibility of an important function for TβRI during cell division. Moreover, TβRI (expression of TGFBR1) was strongly correlated with mitotic spindle and G2/M checkpoint gene sets in prostate cancer (FIGS. 2H, I).

No p-Smad2 was found to localize in the midbody (FIG. 2L), indicating that the canonical TGFβ signaling pathway is not active there. Taken together, these results suggest that TβRI and AURKB co-localize in the midbody and that this co-localization depends on an intact microtubule cytoskeleton.

It was also observed that inhibition of the kinase activity of TβRI by SB505124 suppressed AURKB phosphorylation (FIG. 2L, 2J), suggesting that TβRI kinase activity is important for AURKB activity. Reciprocally, it was observed that His-AURKB phosphorylated glutathione-S-transferase (GST)-TβRI in an in vitro kinase assay (FIG. 2K). There was not found any pSmad2 localization in the cytokinetic structure (e.g. midbody) (FIG. 2L), indicating that the canonical TGFβ signaling pathway is not active there. Taken together, these results suggest that TβRI and AURKB co-localize in the cytokinetic structure (e.g. midbody) and that this co-localization depends on the intact microtubule cytoskeleton.

### Example 3. TRAF6 Promotes Polyubiquitination of AURKB on Lys85 and Lys87

Next, the role of the ubiquitin E3-ligase TRAF6 for the expression of AURKB was investigated. It was observed that TRAF6 knockdown by siRNA led to decreased expression of both H3pS10 and AURKB during the cell cycle, as demonstrated by immunoblotting (FIGS. 3A, B). AURKB was found to associate (precipitate) with TβRI, APPL1 and TRAF6, as determined by a co-immunoprecipitation assay (FIG. 3C).

AURKB has been reported to undergo ubiquitination, which is important for its re-localization from centromeres to microtubules59 and for its involvement in chromatin de-condensation and nuclear envelope formation⁶⁰. The inventors found that AURKB underwent both Lys48-linked (K48-linked) and Lys63-linked (K63-linked) polyubiquitination when PC-3U cells were arrested in mitosis (FIGS. 3D(i) and 3D(ii)). The inventors also investigated if TRAF6 could be autoubiquitinated and activated during mitotic progression after release from double thymidine block. The endogenous TRAF6 was autoubiquitinated 10-12 h after PC-3U cells were released from double thymidine block, i.e., at the time when AURKB is active (FIG. 3E), consistent with current knowledge that autoubiquitination of TRAF6 is enabling its catalytic activity⁶². Knockdown of TRAF6 by siRNA in PC-3U cells suppressed polyubiquitination of AURKB (FIG. 3F). Immunostaining also revealed that endogenous TβRI co-localized with AURKB in a TRAF6-dependent manner in both PC-3U and MEF cell lines (FIGS. 3G, 3H).

The consensus pattern of ubiquitination by TRAF6, i.e. -(hydrophobic)-K-(hydrophobic)-X-X-(hydrophobic)-(polar)-(hydrophobic)-(polar)-(hydrophobic), in which K is the ubiquitinated site and X is any other amino acid⁶³ is found in AURKB (⁸⁴GKGKFGNVYL) (SEQ ID NO: 23), and is conserved among different species (FIG. 3I). To investigate if K85 and/or K87 in AURKB is/are ubiquitinated and, if so, its functional consequence(s), the inventors generated mutants in which Lys85 and/or Lys87 were mutated to arginine residues, the inventors were able to show that the ubiquitination of AURKB was indeed decreased in these mutants (FIGS. 3J(i) and 3J(ii)). Interactions between TRAF6 and the AURKB mutants K85, K85/K87, and to lesser extent K87, were also decreased compared to the interaction with wild-type AURKB, as determined by a co-immunoprecipitation assay (FIG. 3K). Moreover, the phosphorylation of H3 at S10 by AURKB was decreased in cells overexpressing the AURKB K85/87R double mutant (FIGS. 3L(i) and 3L(ii)), suggesting that ubiquitination of AURKB affects its kinase activity. As K85 and K87 are localized in the glycine-rich loop of AURKB, which binds ATP, the inventors investigated the AURKB mutants in an in vitro kinase assay. Both the single mutants and the double K85/87R mutant were found to incorporate radioactive phosphate (FIG. 2M(i)) demonstrating that these mutations did not interfere with binding of ATP. To investigate whether AURKB mutants are intrinsically defective in kinase activity, an in vitro kinase assay using recombinant histone H3 as substrate was performed. All AURKB wild-type and mutants, except the kinase dead K106R which served as control for the experiment, could phosphorylate histone H3 at Ser10, thereby demonstrating conserved intrinsic activity of AURKB mutants (FIG. 3M(ii)).

Of interest, TβRI did not localize to cytokinetic structures when cells overexpressed the AURKB mutants (FIG. 3N), suggesting that ubiquitination of AURKB is required for the recruitment of TβRI in cytokinetic structure (midbodies). Double AURKB mutant (K85/87R)-expressing cells showed less 4N DNA content, compared to wild-type, supporting the biological relevance of polyubiquitination of AURKB on K85 and K87 during replication of the cells (FIG. 3O). Overall, these results support the notion that TRAF6 is autoubiquitinated during mitotic progression and that TRAF6-mediated ubiquitination of AURKB on K85/K87 contributes to its activity and controls the localization of TβRI in the cytokinetic structure during cell division.

### Example 4. Expression of AURKB, and AURKB-TβRI Complex Formation Correlate With Poor Prognosis in Several Tumor Types

Of note, high expression of AURKB mRNA also correlated with poor prognosis in prostate cancer, ccRCC, and lung adenocarcinoma (FIG. 4H, I, J). AURKB expression correlated with the degree of malignancy of prostate cancer, as determined by the Gleason score, based on histopathological scoring in prostate cancer samples (a higher Gleason score indicates more aggressive disease) (FIG. 4G).

To investigate the importance of AURKB and TβRI for cancer progression, the inventors next determined their activity, expression, and complex formation in clinically derived samples. By using an in situ proximity ligation assay (PLA), it was investigated whether Lys63-linked K63-linked) polyubiquitination of AURKB could be visualized in tissues from patients with prostate cancer, clear cell renal cancer (ccRCC) or lung cancer (adenocarcinoma). They observed a high number of Lys63-linked polyubiquitinated AURKB molecules in all three cancer types compared with corresponding normal tissues (FIGS. 4A(i) and 4A(ii)). Moreover, by in situ PLA they also identified a significantly higher number of AURKB and TβRI complexes in sections from patients with aggressive prostate cancer compared to those from patients with less aggressive disease (FIG. 4B) in normal prostate tissues almost no signals were observed (FIG. 4C).

To further investigate expression of genes of interest in different prostate cancer types, bioinformatics analysis was performed using a public database (FIG. 4D(i)-(v)). The expression of both AURKA and AURKB was higher in CRPC-neuroendocrine (CRPC-NE) than in CRPC-adenocarcinoma (CRPC-Adeno) consistent with the observation that CRPC-NE patients have a poor prognosis (FIG. 4D(iii). Furthermore, the expression of AURKB, correlated to the expression of TGFBR1 in both CRPC-NE and CRPC-Adeno (FIG. 4D(iv)). The relative expression of TGFBR1, AURKA, AURKB, TRAF6, VPS4A/B and APPL1/2 in CRPC-NE and CRPC-Adeno including both primary tumors and metastases is also shown (FIGS. 4D(i) and (ii). Interestingly, the expression of APPL1 and AURKA correlated with TGFBR1 in CRPC-NE but not in CRPC-Adeno (Fig. S4D(v)).

It has been reported that in several lung cancer and breast cancer cell lines, loss of RB1 makes cells hyper-dependent on AURKB for their survival⁶⁵. The inventors therefore investigated the expression of RB1 and AURKB in prostate cancer tissues. The inventors found that RB1 is deleted in 10% of prostate cancers (FIG. 4E) and intriguingly, that expression of AURKB is negatively correlated with RB1 in prostate cancer (FIG. 4F), including in neuroendocrine prostate cancer (FIG. 4D(iv)). The expression of both AURKA and AURKB was higher in CRPC-neuroendocrine (CRPC-NE) than in CRPC-adenocarcinoma (CRPC-Adeno), consistent with the observation that CRPC-NE patients have a poor prognosis (FIG. 4D(iii)). Furthermore, the expression of AURKB, correlated to the expression of TGFBR1 in both CRPC-NE and CRPC-Adeno (FIG. 4D(iii)). The relative expression of TGFBR1, AURKA, AURKB, TRAF6, VPS4A/B and APPL1/2 in CRPC-NE and CRPC-Adeno including both primary tumors and metastases is also shown (Figure D(i) and (ii)). Interestingly, the expression of APPL1 and AURKA correlated with TGFBR1 in CRPC-NE but not in CRPC-Adeno (FIG. 4D(v)).

### Example 5. APPL Proteins, TβRI and TRAF6 Affects Cell Growth and Survival

TβRI associates with the endocytic adaptor protein APPL1, which has a role in cell proliferation and survival. Because the interaction between APPL1 and TβRI is important during cancer progression, whether APPL proteins affect proliferation or survival of PC-3U cells was investigated. For this purpose, the MTT (3-(4,5-dimethylthiazol-2-yl)-2,5-diphenyltetrazolium bromide) assay, which measures relative cell number, was used. The results showed that knockdown of APPL1/2 led to a decrease in cell numbers, suggesting that APPL1/2 is needed for cell proliferation or survival (viability) (FIG. 5A).

To further investigate the possible role of APPL1/2 in cell survival, the apoptotic cells was quantified and more of them was found to be in TβRI and APPL1/2 knockdown cell cultures than in controls (FIG. 5B). For comparison, the role of APPL1/2 in the cellular response to epidermal growth factor (EGF) was investigated, which promotes cell proliferation and facilitates nuclear translocation of APPL proteins⁴⁵. Knockdown of APPL1/2 with siRNA resulted in reduced cell numbers when compared with PC-3U control cells treated with EGF (FIG. 5C), suggesting that APPL proteins are important for proliferation or survival of EGF-stimulated cells, consistent with the observation of increased APPL1 gene expression and protein expression during the initiation and progression of prostate cancer. Reduced cell numbers in TRAF6 and TβRI knockdown cultures of PC-3U cells was also observed, suggesting that TRAF6 and TβRI are required for cell proliferation or survival (FIG. 5D).

In summary, the inventors provide a method for identifying patients having a cancer type associated with the non-canonical TGFβ-induced signaling pathways involving cleavage of transforming growth factor β type I receptor (TβRI).

### Discussion

The inventors have previously identified a cancer-specific signaling pathway in which TβRI undergoes proteolytic cleavage in a TRAF6-dependent manner, generating TβRI-ICD which enters the nucleus when TβRI is polyubiquitinated by TRAF6 on residue K178¹³⁻¹⁵. They have also reported that APPL1 interacts with TβRI-ICD via its C-terminus and that the complex traffics to the nucleus via microtubules in a TRAF6-dependent manner¹⁶. Once in the nucleus, TβRI-ICD induces the expression of TβRI and other genes by binding to their promoter regions¹⁴.

Here, AURKB was identified as a target gene for the APPL1/APPL2-dependent pathway in CRPC cells in vitro. TRAF6 was found to be autoubiquitinated in CRPC cells during mitotic progression and to contribute to AURKB kinase activity through K63-linked polyubiquitination on K85/K87 in a conserved glycine-rich part of AURKB. Moreover, the inventors surprisingly found that APPL1 and TβRI-ICD formed a complex with AURKB during mitosis and cytokinesis in CRPC cells. In addition, knockdown of APPL1, TRAF6 or TGFBR1 inhibited proliferation or survival of CRPC cells, suggesting that they are required for growth of CRPC in vitro.

Mitosis is an extraordinarily complex and highly controlled biological process, in which members of the Aurora kinase family have been shown to be required for chromosomal segregation^{41,75,76}. Without being bound by theory, the inventors hypothesize that TβRI-ICD acts together with AURKB to take part in regulation of mitosis and cytokinesis in a TRAF6-dependent manner, involving polyubiquitination of AURKB on K85 and K87. Double mutation of K85 and K87 suppressed the kinase activity of AURKB, suggesting that ubiquitination of these residues contributes to its kinase activity. However, mutation of the two lysine residues did not prevent autophosphorylation of AURKB. These lysine residues are located in the conserved glycine-rich motif G-X-G-X-X-X-G in subdomain I of the AURKB kinase, K85 being located after the first glycine residue and K87 after the second⁷⁷. Importantly, TRAF6 was found to be autoubiquitinated, which is consistent with its activation during mitotic progression at the same time as AURKB is active, in agreement with our hypothesis that active TRAF6 has an impact on AURKB to regulate proliferation of cancer cells.

By confocal imaging we found that APPL1 and AURKB, as well as AURKB and TβRI, colocalized in midbodies during mitosis and cytokinesis. The co-localization of AURKB and TβRI is dependent on TRAF6. Moreover, by co-immunoprecipitation AURKB was shown to interact with APPL1, TβRI and TRAF6 (FIG. 3C). AURKB was found to bind to all three domains of APPL1 (FIG. 1O), while TβRI binds to the PTB-domain of APPL1. It is possible that these interactions are dynamic during mitotic progression and cytokinesis, and the precise constitution of these complexes over time remains to be determined. However, our data suggest that AURKB and TRAF6 associated during mitotic progression to contribute to AURKB activity, and that during late telophase and cytokinesis APPL1, AURKB and TβRI localized in midbodies. Moreover, TβRI localization to midbodies was dependent on K63-linked polyubiquitination on K85 and K87 of AURKB, suggesting that TβRI associated with ubiquitinated AURKB (FIG. 6).

Earlier work has shown that knockdown of AURKB in LnCaP, a human androgen-dependent prostate cancer cell line, does not affect tumor cell survival. In contrast, knockdown of AURKB in the more aggressive, androgen-independent PC3 cells results in apoptosis in vitro and reduced tumor growth in a xenograft nude mouse model in vivo⁸¹, suggesting an important role for AURKB in androgen-independent prostate cancer cells. A previous study described AURKB-related tumor-promoting and pro-survival effects in CPRC⁸². With this result and the current findings that the TβR1-APPL1 pathway controls AURKB expression and that TβRI interacts with AURKB, the inventors hypothesise that TβRI promotes cell proliferation in part through its role during cytokinesis and cell division. Thus, the growth-inhibitory effect transduced by the canonical TβRI-Smad signaling pathway in normal epithelial cells is distinct from the role of TβRI-ICD in complex with AURKB during mitotic progression and cytokinesis, as reported herein. The observation that knockdown of TβRI led to multinucleation of cancer cells underscores the functional role of TβRI in cytokinesis of cancer cells.

AURKB is frequently overexpressed in various cancers, including prostate cancer. Errors in mitosis can lead to genome instability, which is an important hallmark of tumorigenesis⁸³. As noted, Aurora kinases are involved in multiple steps of mitosis, including centrosome maturation, bipolar spindle assembly, chromosome condensation, alignment, and cytokinesis. Because of their specific roles in regulating mitosis, they are target candidates in cancer treatment, with inhibitors being tested in clinical trials^{30,31,41}. Higher expression of AURKB also indicated more aggressiveness of prostate cancer and poorer patient survival (FIG. 4). Although TGFβ inhibits cell proliferation and induces apoptosis in normal epithelial cells, it often promotes the growth of advanced cancers and TβRI kinase inhibitors have been found to block growth of different cancer cell lines. Furthermore, in prostate cancer patients TGFβRI expression was highly correlated with mitotic spindle and G2/M checkpoint (FIG. 2). Moreover, the expression of AURKA and AURKB was higher in CRPC of the neuroendocrine type than in CRPC adenocarcinoma, consistent with the poor prognosis for patients with CRPC of the neuroendocrine type (FIG. 4). The amount of TβRI and AURKB complexes were more frequently observed in sections from prostate cancer patients with higher Gleason score, which indicates more aggressive disease (FIG. 4). In summary, the present data supports the hypothesis that AURKB and TβRI forms a functional complex during cell mitosis and cytokinesis to take part in cell proliferation and that TRAF6-induced ubiquitination of AURKB plays an important role, since the AURKB K85 and K87R mutants did not recruit TβRI to midbodies (FIG. 3).

Taken together, the findings presented here demonstrate a previously unknown function of TβRI in regulating cancer cell proliferation, i.e., through interaction with AURKB when the cells enter mitosis. This function is clearly distinct from the well-known function of TβRI as an upstream regulator of transcriptional responses via the canonical TGFβ-Smad signaling pathway, in response to TGFβ. TRAF6, which associates with TβRI, causes polyubiquitination of AURKB on specific residues (Lys85 and Lys87) (K85 and K87), thereby contributing to AURKB activity as measured by H3pS10 (FIG. 6). The identification of a key function for the TβRI-TRAF6-APPL1-AURKB complex in the cytokinesis of cancer cells provides a basis for developing novel biomarkers and treatment strategies for aggressive cancers that depend on this pathway.

### REFERENCES

1 Esfahani M, Ataei N, Panjehpour M. Biomarkers for Evaluation of Prostate Cancer Prognosis. Asian Pac J Cancer Prev 2015; 16: 2601-2611.
2 Sung H, Ferlay J, Siegel R L, et al. Global Cancer Statistics 2020: GLOBOCAN Estimates of Incidence and Mortality Worldwide for 36 Cancers in 185 Countries. CA Cancer J Clin 2021; 71: 209-249.
3 Massague J. TGFβ in Cancer. Cell 2008; 134: 215-230.
4 Heldin C H, Moustakas A. Signaling Receptors for TGF-β Family Members. Cold Spring Harb Perspect Biol 2016; 8: a022053.
5 Batlle E, Massague J. Transforming Growth Factor-β Signaling in Immunity and Cancer. Immunity 2019; 50: 924-940.
6 Liu S, Ren J, ten Dijke P. Targeting TGFβ signal transduction for cancer therapy. Signal Transduct Target Ther 2021; 6: 8.
7 Liu C, Xu P, Lamouille S, et al. TACE-Mediated Ectodomain Shedding of the Type I TGF-β Receptor Downregulates TGF-β Signaling. Mol Cell 2009; 35: 26-36.
8 Sorrentino A, Thakur N, Grimsby S, et al. The type I TGF-β receptor engages TRAF6 to activate TAK1 in a receptor kinase-independent manner. Nat Cell Biol 2008; 10: 1199-1207.
9 Yamashita M, Fatyol K, Jin C, et al. TRAF6 Mediates Smad-Independent Activation of JNK and p38 by TGF-β. Mol Cell 2008; 31: 918-924.
10 Landstrom M. The TAK1-TRAF6 signalling pathway. Int J Biochem Cell Biol 2010; 42: 585-589.
11 Cheng K K, Lam K S, Wang Y, et al. TRAF6-mediated ubiquitination of APPL1 enhances hepatic actions of insulin by promoting the membrane translocation of Akt. Biochem J 2013; 455: 207-216.
12 Hamidi A, Song J, Thakur N, et al. TGF-β promotes PI3K-AKT signaling and prostate cancer cell migration through the TRAF6-mediated ubiquitylation of p85α. Sci Signal 2017; 10: eaal4186.
13 Mu Y, Sundar R, Thakur N, et al. TRAF6 ubiquitinates TGFβ type I receptor to promote its cleavage and nuclear translocation in cancer. Nat Commun 2011; 2: 330.
14 Gudey S K, Sundar R, Mu Y, et al. TRAF6 Stimulates the Tumor-Promoting Effects of TGFβ Type I Receptor Through Polyubiquitination and Activation of Presenilin 1. Sci Signal 2014; 7: ra2.
15 Sundar R, Gudey S K, Heldin C H, et al. TRAF6 promotes TGFβ-induced invasion and cell-cycle regulation via Lys63-linked polyubiquitination of Lys178 in TGFβ type I receptor. Cell Cycle 2015; 14: 554-565.
16 Song J, Mu Y, Li C, et al. APPL proteins promote TGFβ-induced nuclear transport of the TGFβ type I receptor intracellular domain. Oncotarget 2016; 7: 279-292.
17 Sitaram R T, Mallikarjuna P, Landstrom M, et al. Transforming growth Factor-β promotes aggressiveness and invasion of clear cell renal cell carcinoma. Oncotarget 2016; 7: 35917-35931.
18 David C J, Massague J. Contextual determinants of TGFβ action in development, immunity and cancer. Nat Rev Mol Cell Biol 2018; 19: 419-435.
19 Datto M B, Li Y, Panus J F, et al. Transforming growth factor β induces the cyclin-dependent kinase inhibitor p21 through a p53-independent mechanism. Proc Natl Acad Sci USA 1995; 92: 5545-5549.
20 Seoane J, Pouponnot C, Staller P, et al. TGFβ influences Myc, Miz-1 and Smad to control the CDK inhibitor p15INK4b.Nat Cell Biol 2001; 3: 400-408.
21 Derynck R, Turley S J, Akhurst R J. TGFβ biology in cancer progression and immunotherapy. Nat Rev Clin Oncol 2021; 18: 9-34.
22 Moustakas A, Heldin C H. Mechanisms of TGFβ-Induced Epithelial-Mesenchymal Transition. J Clin Med 2016; 5: 63.
23 Dongre A, Weinberg R A. New insights into the mechanisms of epithelial-mesenchymal transition and implications for cancer. Nat Rev Mol Cell Biol 2019; 20: 69-84.
24 Strutz F, Zeisberg M, Renziehausen A, et al. TGF-β1 induces proliferation in human renal fibroblasts via induction of basic fibroblast growth factor (FGF-2). Kidney Int 2001; 59: 579-592.
25 Matsuyama S, Iwadate M, Kondo M, et al. SB-431542 and Gleevec Inhibit Transforming Growth Factor-β-Induced Proliferation of Human Osteosarcoma Cells. Cancer Res 2003; 63: 7791-7798.
26 Ikushima H, Miyazono K. TGFβ signalling: a complex web in cancer progression. Nat Rev Cancer 2010; 10: 415-424.
27 Sintich S M, Lamm M L, Sensibar J A, et al. Transforming Growth Factor-β1-Induced Proliferation of the Prostate Cancer Cell Line, TSU-Pr1: The Role of Platelet-Derived Growth Factor. Endocrinology 1999; 140: 3411-3415.
28 Wilding G, Zugmeier G, Knabbe C, et al. Differential effects of transforming growth factor β on human prostate cancer cells in vitro. Mol Cell Endocrinol 1989; 62: 79-87.
29 Mallikarjuna P, Raviprakash T S, Aripaka K, et al. Interactions between TGF-β type I receptor and hypoxia-inducible factor-α mediates a synergistic crosstalk leading to poor prognosis for patients with clear cell renal cell carcinoma. Cell Cycle 2019; 18: 2141-2156.
30 Borah N A, Reddy MM. Aurora Kinase B Inhibition: A Potential Therapeutic Strategy for Cancer. Molecules 2021; 26.
31 Mou P K, Yang E J, Shi C, et al. Aurora kinase A, a synthetic lethal target for precision cancer medicine. Exp Mol Med 2021; 53: 835-847.
32 Yoo S, Sinha A, Yang D, et al. Integrative network analysis of early-stage lung adenocarcinoma identifies aurora kinase inhibition as interceptor of invasion and progression. Nat Commun 2022; 13: 1592.
33 Carmena M, Wheelock M, Funabiki H, et al. The chromosomal passenger complex (CPC): from easy rider to the godfather of mitosis. Nat Rev Mol Cell Biol 2012; 13: 789-803.
34 Trivedi P, Stukenberg P T. A Condensed View of the Chromosome Passenger Complex. Trends Cell Biol 2020; 30: 676-687.
35 Adams R R, Wheatley S P, Gouldsworthy A M, et al. INCENP binds the Aurora-related kinase AIRK2 and is required to target it to chromosomes, the central spindle and cleavage furrow. Curr Biol 2000; 10: 1075-1078.
36 Bishop J D, Schumacher J M. Phosphorylation of the Carboxyl Terminus of Inner Centromere Protein (INCENP) by the Aurora B Kinase Stimulates Aurora B Kinase Activity. J Biol Chem 2002; 277: 27577-27580.
37 Elkins J M, Santaguida S, Musacchio A, et al. Crystal structure of human Aurora B in complex with INCENP and VX-680. J Med Chem 2012; 55: 7841-7848.
38 Abdul Azeez K R, Chatterjee S, Yu C, et al. Structural mechanism of synergistic activation of Aurora kinase B/C by phosphorylated INCENP. Nat Commun 2019; 10: 3166.
39 Goldenson B, Crispino J D. The aurora kinases in cell cycle and leukemia. Oncogene 2015; 34: 537-545.
40 Fuller B G, Lampson M A, Foley E A, et al. Midzone activation of aurora B in anaphase produces an intracellular phosphorylation gradient. Nature 2008; 453: 1132-1136.
41 Tang A, Gao K, Chu L, et al. Aurora kinases: novel therapy targets in cancers. Oncotarget 2017; 8: 23937-23954.
42 Fransson S, Hansson M, Ruuth K, et al. Intragenic anaplastic lymphoma kinase (ALK) rearrangements: translocations as a novel mechanism of ALK activation in neuroblastoma tumors. Genes Chromosomes Cancer 2015; 54: 99-109.
43 Franzén P, Ichijo H, Miyazono K. Different signals mediate transforming growth factor-β1-induced growth inhibition and extracellular matrix production in prostatic carcinoma cells. Exp Cell Res 1993; 207: 1-7.
44 Hengeveld R C, Hertz N T, Vromans M J, et al. Development of a chemical genetic approach for human Aurora B kinase identifies novel substrates of the chromosomal passenger complex. Mol Cell Proteomics 2012; 11: 47-59.
45 Miaczynska M, Christoforidis S, Giner A, et al. APPL proteins link Rab5 to nuclear signal transduction via an endosomal compartment. Cell 2004; 116: 445-456.
46 Erdmann K S, Mao Y, McCrea H J, et al. A role of the Lowe syndrome protein OCRL in early steps of the endocytic pathway. Dev Cell 2007; 13: 377-390.
47 Yu G, Wang LG, Han Y, et al. clusterProfiler: an R package for comparing biological themes among gene clusters. OMICS 2012; 16: 284-287.
48 Liberzon A, Birger C, Thorvaldsdottir H, et al. The Molecular Signatures Database Hallmark Gene Set Collection. Cell Syst 2015; 1: 417-425.
49 Grossman R L, Heath A P, Ferretti V, et al. Toward a Shared Vision for Cancer Genomic Data. N Engl J Med 2016; 375: 1109-1112.
50 Colaprico A, Silva T C, Olsen C, et al. TCGAbiolinks: an R/Bioconductor package for integrative analysis of TCGA data. Nucleic Acids Res 2016; 44: e71.
51 Kassambara A. ggpubr: ‘ggplot2’ Based Publication Ready Plots. R package version 0.4.0. 2020. https://CRAN.R-project.org/package=ggpubr.
52 Beltran H, Prandi D, Mosquera J M, et al. Divergent clonal evolution of castration-resistant neuroendocrine prostate cancer. Nat Med 2016; 22: 298-305.
53 R Core Team. R: A language and environment for statistical computing. R Foundation for Statistical Computing. 2013. http://www.R-project.org/.
54 Rafatmanesh A, Behjati M, Mobasseri N, et al. The survivin molecule as a double-edged sword in cellular physiologic and pathologic conditions and its role as a potential biomarker and therapeutic target in cancer. J Cell Physiol 2020; 235: 725-744.
55 Gudey S K, Sundar R, Heldin C H, et al. Pro-invasive properties of Snail1 are regulated by sumoylation in response to TGFβ stimulation in cancer. Oncotarget 2017; 8: 97703-97726.
56 Liu Z, Xiao T, Peng X, et al. APPLs: More than just adiponectin receptor binding proteins. Cell Signal 2017; 32: 76-84.
57 Fabbro M, Zhou B B, Takahashi M, et al. Cdk1/Erk2- and Plk1-dependent phosphorylation of a centrosome protein, Cep55, is required for its recruitment to midbody and cytokinesis. Dev Cell 2005; 9: 477-488.
58 Mora-Bermudez F, Gerlich D, Ellenberg J. Maximal chromosome compaction occurs by axial shortening in anaphase and depends on Aurora kinase. Nat Cell Biol 2007; 9: 822-831.
59 Afonso O, Figueiredo A C, Maiato H. Late mitotic functions of Aurora kinases. Chromosoma 2017; 126: 93-103.
60 Ramadan K, Bruderer R, Spiga F M, et al. Cdc48/p97 promotes reformation of the nucleus by extracting the kinase Aurora B from chromatin. Nature 2007; 450: 1258-1262.
61 Honda R, Korner R, Nigg E A. Exploring the functional interactions between Aurora B, INCENP, and survivin in mitosis. Mol Biol Cell 2003; 14: 3325-3341.
62 Wang C, Deng L, Hong M, et al. TAK1 is a ubiquitin-dependent kinase of MKK and IKK. Nature 2001; 412: 346-351.
63 Jadhav T, Geetha T, Jiang J, et al. Identification of a consensus site for TRAF6/p62 polyubiquitination. Biochem Biophys Res Commun 2008; 371: 521-524.
64 Kim W, Bennett E J, Huttlin E L, et al. Systematic and quantitative assessment of the ubiquitin-modified proteome. Mol Cell 2011; 44: 325-340.
65 Oser M G, Fonseca R, Chakraborty A A, et al. Cells Lacking the RB1 Tumor Suppressor Gene Are Hyperdependent on Aurora B Kinase for Survival. Cancer Discov 2019; 9: 230-247.
66 Alanee S, Moore A, Nutt M, et al. Contemporary Incidence and Mortality Rates of Neuroendocrine Prostate Cancer. Anticancer Res 2015; 35: 4145-4150.
67 Tsaur I, Heidegger I, Kretschmer A, et al. Aggressive variants of prostate cancer—Are we ready to apply specific treatment right now? Cancer Treat Rev 2019; 75: 20-26.
68 Deepa S S, Dong L Q. APPL1: role in adiponectin signaling and beyond. Am J Physiol Endocrinol Metab 2009; 296: E22-36.
69 Johnson I R D, Parkinson-Lawrence E J, Keegan H, et al. Endosomal gene expression: a new indicator for prostate cancer patient prognosis? Oncotarget 2015; 6: 37919-37929.
70 Starczynowski D T, Lockwood W W, Delehouzee S, et al. TRAF6 is an amplified oncogene bridging the RAS and NF-κB pathways in human lung cancer. Journal of Clinical Investigation 2011; 121: 4095-4105.
71 Aripaka K, Gudey S K, Zang G, et al. TRAF6 function as a novel co-regulator of Wnt3a target genes in prostate cancer. EBioMedicine 2019; 45: 192-207.
72 The Human Protein Atlas. TGFBR1 in breast cancer.

https://www.proteinatlas.org/ENSG00000106799-TGFBR1/pathology/breast+cancer (accessed May 16 2022).
73 Yang W L, Wang J, Chan C H, et al. The E3 ligase TRAF6 regulates Akt ubiquitination and activation. Science 2009; 325: 1134-1138.
74 Rong Y, Wang D, Wu W, et al. TRAF6 is over-expressed in pancreatic cancer and promotes the tumorigenicity of pancreatic cancer cells. Med Oncol 2014; 31: 260.
75 Krenn V, Musacchio A. The Aurora B kinase in chromosome bi-orientation and spindle checkpoint signaling. Front Oncol 2015; 5: 225.
76 Willems E, Dedobbeleer M, Digregorio M, et al. The functional diversity of Aurora kinases: a comprehensive review. Cell Div 2018; 13: 7.
77 Hanks S K, Hunter T. The eukaryotic protein kinase superfamily: kinase (catalytic) domain structure and classification. FASEB J 1995; 9: 576-596.
78 Bose A, Sudevan S, Rao V J, et al. Haploinsufficient tumor suppressor Tip60 negatively regulates the oncogenic Aurora B kinase. J Biosci 2019; 44: 147.
79 Cheetham G M, Knegtel R M, Coll J T, et al. Crystal structure of Aurora-2, an oncogenic serine/threonine kinase. J Biol Chem 2002; 277: 42419-42422.
80 Yasui Y, Urano T, Kawajiri A, et al. Autophosphorylation of a newly identified site of Aurora-B is indispensable for cytokinesis. J Biol Chem 2004; 279: 12997-13003.
81 Addepalli M K, Ray K B, Kumar B, et al. RNAi-mediated knockdown of AURKB and EGFR shows enhanced therapeutic efficacy in prostate tumor regression. Gene Ther 2010; 17: 352-359.
82 Chieffi P, Cozzolino L, Kisslinger A, et al. Aurora B expression directly correlates with prostate cancer malignancy and influence prostate cell proliferation. Prostate 2006; 66: 326-333.
83 Hanahan D, Weinberg R A. Hallmarks of cancer: the next generation. Cell 2011; 144: 646-674.

1. A method for diagnosing cancer in a subject, the method comprising the steps of:
providing a biological test sample from the subject; and
determining the presence or absence of a first biomarker, a second biomarker, and a third biomarker, wherein said biomarkers are: Aurora kinase B (AURKB), Adaptor Protein, Phosphotyrosine Interacting With PH Domain And Leucine Zipper 1 (APPL1), and TGFβ receptor type 1 (TβR1), in the biological test sample;
wherein the co-localization of all three biomarkers in the biological test sample is indicative of cancer in the subject.

2. The method according to claim 1, further comprising determining the presence or absence of a fourth biomarker, wherein said biomarker is TNF receptor associated factor 6 (TRAF6) in the biological test sample, wherein the co-localization of all four biomarkers in the biological test sample in the biological sample is indicative of cancer in the subject.

3. A method for diagnosing and/or prognosing aggressive cancer in a subject, the method comprising the steps of:
providing a biological test sample from the subject;
determining the presence or absence of a first biomarker, a second biomarker, and a third biomarker, wherein said biomarkers are: Aurora kinase B (AURKB), Adaptor Protein, Phosphotyrosine Interacting With PH Domain And Leucine Zipper 1 (APPL1), and TGFβ receptor type 1 (TβR1), in said test sample; and
wherein the co-localization of all three biomarkers in the biological sample is indicative of aggressive cancer in the subject.

4. The method according to claim 3, further comprising determining the presence or absence of a fourth biomarker, wherein said biomarker is TNF receptor associated factor 6 (TRAF6) in the biological test sample, wherein the co-localization of all four biomarkers in the biological test sample is indicative of aggressive cancer in the subject.

5. The method according to claim 1, wherein Aurora kinase B (AURKB) is ubiquitinated.

6. The method according to claim 5, wherein AURKB is ubiquitinated at one or both lysine residues corresponding to Lysine 85 (K85) and/or Lysine 87 (K87) of human AURKB (SEQ ID NO: 1).

7. The method according to claim 1, wherein the cancer is associated with and/or mediated by the proteolytic cleavage of transforming growth factor β type I receptor (TβRI).

8. The method according to claim 1, wherein the cancer is a solid tumour.

9. The method according to claim 8, wherein the solid tumour comprises prostate cancer, renal carcinoma, lung cancer, kidney cancer, gastric cancer, bladder carcinoma, breast cancer, endometrial cancer, ovarian cancer, or colorectal cancer.

10. The method according to claim 9, wherein the prostate cancer is castration-resistant prostate cancer (CRPC).

11. The method according to claim 1, wherein the test sample is a tissue sample, such as a biopsy from a tumour.

12. The method according to claim 1, wherein the presence or absence of Aurora kinase B (AURKB), Adaptor Protein, Phosphotyrosine Interacting With PH Domain And Leucine Zipper 1 (APPL1), TGFβ receptor type 1 (TβR1) and/or TNF receptor associated factor 6 (TRAF6) is determined by detecting the biomarker protein; and/or detecting a biological activity of the biomarker protein.

13. The method according to claim 1, wherein determining the presence and/or absence of the biomarkers in step (b) is performed using a method selected from the group consisting of immunohistochemistry, immunocytochemistry, immunoprecipitation (IP), ELISA techniques (single or mulitplex), radioimmunoassay (RIA), immunoradiometric assays (IRMA) and immunoenzymatic assays (IEMA), including sandwich assays using monoclonal and/or polyclonal antibodies, in situ proximity ligation assay (PLA), enzymatic methods, image analysis, mass spectrometry, aptamers, Bio-Layer Interferometry (BLI), Surface plasmon resoncance (SPR), Multiplex assay (MSD, Mesoscale discovery), or by indicator substances that bind to Aurora kinase B (AURKB), Adaptor Protein, Phosphotyrosine Interacting With PH Domain And Leucine Zipper 1 (APPL1), TGFβ receptor type 1 (TβR1), optionally TGFβ receptor type 1 intracellular domain (TβR1-ICD), and TNF receptor associated factor 6 (TRAF6).

14. The method according to claim 1, wherein in the event that the subject is diagnosed with cancer and/or aggressive cancer, the method further comprises the step of:
administering a cancer therapy to the subject, optionally wherein the cancer therapy comprises one or more of surgery, chemotherapy, immunotherapy, chemoimmunotherapy and thermochemotherapy.

15. A method for determining the Gleason score (GS) in a subject suffering from, or suspected to be suffering from prostate cancer, as being either (i) GS≤6 or 7 (3+4); or (ii) GS 7 (4+3) or ≥8, the method comprising the steps of:
a) providing a biological test sample from the subject;
b) assessing the amount of a complex comprising Aurora kinase B (AURKB) and TGFβ receptor type 1 (TβR1); and
c) comparing the amount of the complex in (b) with the amount of a complex comprising Aurora kinase B (AURKB) and TGFβ receptor type 1 (TβR1) from a reference sample that is known to have a GS of either (i) GS≤6 or 7 (3+4); or (ii) GS 7 (4+3) or ≥8;
wherein the comparison allows the determination of the GS in the subject as being either (i) GS≤6 or 7 (3+4), or (ii) GS 7 (4+3) or ≥8.

16. The method according to claim 15, wherein the complex further comprises Adaptor Protein, Phosphotyrosine Interacting With PH Domain And Leucine Zipper 1 (APPL1).

17. The method according to claim 15, wherein the complex further comprises TNF receptor associated factor 6 (TRAF6).

18. The method according to claim 15, wherein the complex is localised to a cellular structure, such as a cytokinesis structure.

19. The method according to claim 15, wherein AURKB is ubiquitinated at one or both lysine residues corresponding to Lysine 85 (K85) and Lysine 87 (K87) of human AURKB (SEQ ID NO: 1).

20. The method according to claim 15, wherein the TGFβ receptor type 1 (TβR1) is the intracellular domain (TβR1-ICD).

21. An array for determining the presence of cancer in an individual comprising:
(i) a binding agent capable of binding to Aurora kinase B (AURKB) and/or a binding moiety capable of binding selectively to a nucleic acid molecule encoding Aurora kinase B (AURKB);
(ii) a binding agent capable of binding to Adaptor Protein, Phosphotyrosine Interacting With PH Domain And Leucine Zipper 1 (APPL1) and/or a binding moiety capable of binding selectively to a nucleic acid molecule encoding Adaptor Protein, Phosphotyrosine Interacting With PH Domain And Leucine Zipper 1 (APPL1);
(iii) a binding agent capable of binding to TGFβ receptor type 1 (TβR1) and/or a binding moiety capable of binding selectively to a nucleic acid molecule encoding TGFβ receptor type 1 (TβR1); and
(iv) a binding agent capable of binding to TNF receptor associated factor 6 (TRAF6) and/or a binding moiety capable of binding selectively to a nucleic acid molecule encoding TNF receptor associated factor 6 (TRAF6).

22. Kit for the diagnosis and/or prognosis of a cancer in a subject, said kit comprising:
(i) a binding agent capable of binding to Aurora kinase B (AURKB) and/or a binding moiety capable of binding selectively to a nucleic acid molecule encoding Aurora kinase B (AURKB);
(ii) a binding agent capable of binding to Adaptor Protein, Phosphotyrosine Interacting With PH Domain And Leucine Zipper 1 (APPL1) and/or a binding moiety capable of binding selectively to a nucleic acid molecule encoding Adaptor Protein, Phosphotyrosine Interacting With PH Domain And Leucine Zipper 1 (APPL1);
(iii) a binding agent capable of binding to TGFβ receptor type 1 (TβR1) and/or a binding moiety capable of binding selectively to a nucleic acid molecule encoding TGFβ receptor type 1 (TβR1); and
(iv) a binding agent capable of binding to TNF receptor associated factor 6 (TRAF6) and/or a binding moiety capable of binding selectively to a nucleic acid molecule encoding TNF receptor associated factor 6 (TRAF6), and optionally instructions for performing the method of claim 1.

23-26. (canceled)

27. A complex comprising Aurora kinase B (AURKB), Adaptor Protein, Phosphotyrosine Interacting With PH Domain And Leucine Zipper 1 (APPL1), and TGFβ receptor type 1 (TβR1), wherein AURKB is ubiquitinated.

28. The complex according to claim 27, further comprising TNF receptor associated factor 6 (TRAF6).

29. The complex according to claim 27, wherein Aurora kinase B (AURKB) is ubiquitinated at one or both lysine residues corresponding to Lysine 85 (K85) and/or Lysine 87 (K87) of human AURKB (SEQ ID NO: 1).


SEQUENCE LISTING (25 sequences)

SEQ ID NO: 1 - protein, 344 aa, Homo sapiens
  AURKB amino acid sequence
MAQKENSYPWPYGRQTAPSGLSTLPQRVLRKEPVTPSALVLMSRSNVQPTAAPGQKVMENSSGTPDILTRHFTIDDFEIGRPLGKGKFGNVYLAREKKSHFIVALKVLFKSQIEKEGVEHQLRREIEIQAHLHHPNILRLYNYFYDRRRIYLILEYAPRGELYKELQKSCTFDEQRTATIMEELADALMYCHGKKVIHRDIKPENLLLGLKGELKIADFGWSVHAPSLRRKTMCGTLDYLPPEMIEGRMHNEKVDLWCIGVLCYELLVGNPPFESASHNETYRRIVKVDLKFPASVPMGAQDLISKLLRHNPSERLPLAQVSAHPWVRANSRRVLPPSALQSVA

SEQ ID NO: 2 - DNA, 1035 bp, Homo sapiens
  AURKB coding sequence
atggcccagaaggagaactcctacccctggccctacggccgacagacggctccatctggcctgagcaccctgccccagcgagtcctccggaaagagcctgtcaccccatctgcacttgtcctcatgagccgctccaatgtccagcccacagctgcccctggccagaaggtgatggagaatagcagtgggacacccgacatcttaacgcggcacttcacaattgatgactttgagattgggcgtcctctgggcaaaggcaagtttggaaacgtgtacttggctcgggagaagaaaagccatttcatcgtggcgctcaaggtcctcttcaagtcccagatagagaaggagggcgtggagcatcagctgcgcagagagatcgaaatccaggcccacctgcaccatcccaacatcctgcgtctctacaactatttttatgaccggaggaggatctacttgattctagagtatgccccccgcggggagctctacaaggagctgcagaagagctgcacatttgacgagcagcgaacagccacgatcatggaggagttggcagatgctctaatgtactgccatgggaagaaggtgattcacagagacataaagccagaaaatctgctcttagggctcaagggagagctgaagattgctgacttcggctggtctgtgcatgcgccctccctgaggaggaagacaatgtgtggcaccctggactacctgcccccagagatgattgaggggcgcatgcacaatgagaaggtggatctgtggtgcattggagtgctttgctatgagctgctggtggggaacccaccctttgagagtgcatcacacaacgagacctatcgccgcatcgtcaaggtggacctaaagttccccgcttccgtgcccatgggagcccaggacctcatctccaaactgctcaggcataacccctcggaacggctgcccctggcccaggtctcagcccacccttgggtccgggccaactctcggagggtgctgcctccctctgcccttcaatctgtcgcctga

SEQ ID NO: 3 - DNA, 21 bp, Artificial Sequence
  siRNA-resistance construct of APPL1
agagagatggattcagacata

SEQ ID NO: 4 - DNA, 21 bp, Artificial Sequence
  siRNA-resistance construct of APPL2
cagatttatctcacagataac

SEQ ID NO: 5 - DNA, 32 bp, Artificial Sequence
  oJS5
ccccgggaatcaaaacgaattcgccaccatgg

SEQ ID NO: 6 - DNA, 56 bp, Artificial Sequence
  oJS8
cgaattcgccaccatggactacaaagacgatgacgacaaggcccagaaggagaact

SEQ ID NO: 7 - DNA, 33 bp, Artificial Sequence
  oJS17
tgaagaggaccttgagcgccacgatgaaatggc

SEQ ID NO: 8 - DNA, 26 bp, Artificial Sequence
  oJS18
cgcttctgtgcccatgggagcccagg

SEQ ID NO: 9 - DNA, 28 bp, Artificial Sequence
  oJS9
caaacttgcctctgcccagaggacgccc

SEQ ID NO: 10 - DNA, 33 bp, Artificial Sequence
  oJS10
gtacacgtttccaaacctgcctttgcccagagg

SEQ ID NO: 11 - DNA, 35 bp, Artificial Sequence
  oJS11
acacgtttccaaacctgcctctgcccagaggacgc

SEQ ID NO: 12 - DNA, 34 bp, Artificial Sequence
  oYZ86
accccaccaaatttcctgaattctgcagtcgacg

SEQ ID NO: 13 - DNA, 39 bp, Artificial Sequence
  oYZ87
cggactcagatctcgagtggttaatcgaaatttaacccg

SEQ ID NO: 14 - DNA, 49 bp, Artificial Sequence
  oYZ88
ctgtacaataaacaacatatctaaacaaatagaattctgcagtcgacgg

SEQ ID NO: 15 - DNA, 45 bp, Artificial Sequence
  oYZ91
gtccggactcagatctcgagtgattcttcatcagttatttattgt

SEQ ID NO: 16 - DNA, 32 bp, Artificial Sequence
  oYZ92
ggatcgtagggcatcagaattctgcagtcgac

SEQ ID NO: 17 - RNA, 19 nt, Artificial Sequence
  On TARGET plus APPL1 - No. 1 target sequence
ggaaauggacagugauaua

SEQ ID NO: 18 - RNA, 19 nt, Artificial Sequence
  On TARGET plus APPL1 - No. 2 target sequence
gaucugagucuacaaauuu

SEQ ID NO: 19 - RNA, 19 nt, Artificial Sequence
  On TARGET plus APPL2 - No. 1 target sequence
agaucuaccugaccgacaa

SEQ ID NO: 20 - RNA, 19 nt, Artificial Sequence
  On TARGET plus APPL2 - No. 2 target sequence
gcggaaaagaugcgggugu

SEQ ID NO: 21 - RNA, 19 nt, Artificial Sequence
  T?RI siRNA - target sequence
cauauugcugcaaccagga

SEQ ID NO: 22 - RNA, 19 nt, Artificial Sequence
  SMART pool TRAF6 siRNA, and siGENOME non-targeting control siRNA #1- target sequence
uagcgacuaaacacaucaa

SEQ ID NO: 23 - protein, 10 aa, Artificial Sequence
  AURKB consensus sequence
GKGKFGNVYL

SEQ ID NO: 24 - protein, 10 aa, Artificial Sequence
  SITE 1: hydrophobic amino acid
  *K*KX*&*&* consensus sequence
  SITE 2: K=acceptor lysine, at least one of the lysine residues therein is ubiquitinated
  SITE 3: hydrophobic amino acid
  SITE 4: K=acceptor lysine, at least one of the lysine residues therein is ubiquitinated
  SITE 5: any amino acid
  SITE 6: hydrophobic amino acid
  SITE 7: polar amino acid
  SITE 8: hydrophobic amino acid
  SITE 9: polar amino acid
  SITE 10: hydrophobic amino acid
XKXKXXXXXX

SEQ ID NO: 25 - protein, 10 aa, Artificial Sequence
  SITE 1: hydrophobic amino acid residue
  AURKB consensus pattern - Figure 3I
  SITE 2: K is the acceptor lysine residue
  SITE 3: hydrophobic amino acid residue
  SITE 4: any amino acid residue
  SITE 5: any amino acid residue
  SITE 6: hydrophobic amino acid residue
  SITE 7: polar amino acid residue
  SITE 8: hydrophobic amino acid residue
  SITE 9: polar amino acid residue
  SITE 10: hydrophobic amino acid residue
XKXXXXXXXX